(19) United States
(12) Patent Application Publication
(10) Pub. No.: US 2003/0228681 A1
(43) Pub. Date: Dec. 11, 2003

(54) Analyte sensor

(75) Inventors: Rosalyn Ritts, Princeton, NJ (US); Hoi-Cheong Steve Sun, Dayton, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: PowerZyme, Inc., Monmouth Junction, NJ

(21) Appl. No.: 10/407,794

(22) Filed: Apr. 4, 2003

Related U.S. Application Data
(60) Provisional application No. 60/370,500, filed on Apr. 5, 2002.

(51) Int. Cl. C12M 1/34

(52) U.S. Cl. 435/287.2

(57) ABSTRACT

The present invention relates to analyte sensors including a first compartment adapted for introduction of a sample potentially containing the targeted analyte, and a second compartment separated from the first compartment by a barrier, wherein the analyte interacts with a component in the first compartment, or a polypeptide associated with the barrier, resulting in the transport of a species across the barrier, the transported species or a derivative thereof being detected by a detector, thereby indicating the presence of the analyte.

### CROSS-REFERENCE TO RELATED APPLICATIONS

This application claims priority under 35 U.S.C. § 119(e) based upon provisional application No. 60/370,500 filed on Apr. 5, 2002, the disclosure of which is hereby incorporated by reference.

### BACKGROUND OF THE INVENTION

Analytical sensors are useful in a wide range of applications. In the medical and pharmaceutical fields, they can be employed in clinical diagnostics and new drug discovery. They can also play a part in personal safety and national security where used to detect bio-terrorism agents or the release of toxic chemical agents. They may be used in an industrial setting to protect workers from exposure to particularly toxic chemicals by providing an early warning of exposure.

For an analyte sensor to be effective, it must have a certain degree of sensitivity and specificity; detecting particular species or classes of analytes. Preferably, the sensors should be adaptable to a wide range of environments; stable, e.g., providing long-lasting sensor capabilities; inexpensive, e.g., preferably reusable; and portable. Improvements in one or more of these characteristics is therefore desirable. It is an objective of the present invention to provide a novel arrangement for analyte sensors which incorporates or improves upon the desired features of traditional analyte sensors.

### SUMMARY OF THE INVENTION

The present invention relates to dual- and multi-chambered analyte sensors, wherein an analyte or analyte derivative species is transported across a barrier separating the chambers in order to effect detection of the analyte.

In one embodiment, the analyte is introduced into a first compartment and a barrier separates the first compartment from a second compartment. Additionally, the first compartment contains at least one component to interact with the analyte, resulting in the transport of a species across the barrier. For purposes of the present invention, the transported species can be, e.g., an electron, proton, atom, molecule or ion, including anion, cation or ion of specific valency. The transported species or a derivative of the transported species is detected, thus indicating the presence of the analyte.

In another embodiment, the barrier includes at least one layer of a synthetic polymer, the layer being relatively proton impermeable, yet capable of participating in the transport of protons across the barrier.

In yet another embodiment, the barrier includes a biological membrane associated with a polypeptide, wherein the polypeptide is capable of participating in the transport of a species across the barrier upon interaction with the analyte or a derivative of the analyte. The transported species or a derivative of the transported species is detected, thus indicating the presence of the analyte. Preferably, the detector of this embodiment is either (i) not attached to the membrane, or (ii) separated from the membrane by 50 nm or more.

Additional components can be provided in one or more of the chambers in order to facilitate interaction of the analyte and a polypeptide and/or the transported species and detector.

In a particularly preferred embodiment, the barrier includes a biocompatible membrane having at least one layer of a synthetic polymer, the membrane being associated with at least one polypeptide. The polypeptide is capable of participating in the transport of a species across the barrier upon interaction with the analyte or a derivative of the analyte. The transported species or a derivative of the transported species is detected, thereby detecting the presence of the analyte.

In another preferred embodiment, the synthetic polymer includes at least one block copolymer and at least one non-block polymer or copolymer and the polypeptide is capable of transporting protons across the barrier.

Furthermore, additional components can be provided in one or more of the chambers in order to facilitate interaction of the analyte and the polypeptide and/or the transported species and detector.

In yet another embodiment, the barrier comprises at least one layer of a synthetic polymer material that is proton impermeable, in a preferred aspect substantially proton impermeable, yet capable of participating in the transport of protons across the barrier. Protons transported across the barrier are detected, thereby detecting the presence of the analyte.

Particularly preferred detectors are adapted to detect electrical current; however, other types of detectors are contemplated.

In another embodiment a detection device includes an analyte sensor and further comprises a separation module adapted to separate a sample to be analyzed into at least two sample components. A transfer element is adapted to transfer at least one of the separated components to the first compartment of an analyte sensor to detect the presence of the analyte. The sample may undergo multiple separations, which may include more than one type of separation device.

Another embodiment includes an array of analyte sensors. The array includes more than one analyte sensor and is adapted to detect different analytes; alternatively, it can employ various sensors and/or detectors for detecting the same analyte.

Yet another embodiment of the present invention relates to methods for detecting the presence of one or more analytes using the various analyte sensors of the present invention.

### BRIEF DESCRIPTION OF THE DRAWINGS

FIG. 1A illustrates one embodiment of an analyte sensor of the present invention.

FIG. 1B is a side view of a barrier including membranes.

FIG. 1C is a close up view of transmembrane proteins associated with a membrane of the analyte sensor shown in FIGS. 1A-B.

FIG. 1D shows another embodiment of an analyte sensor of the present invention.

FIGS. 2A-C illustrate additional embodiments of analyte sensors of the present invention.

FIG. 3 illustrates an array of analyte sensors.

FIG. 4 illustrates a biological membrane type sensor of the prior art.

FIGS. 5A-5B illustrate the anchoring of a membrane to a barrier.

FIGS. 6A-B show fabric/mesh supported membranes.

FIG. 7 shows a separation device with an attached analyte sensor.

FIG. 8 is a cross-sectional view of a PTM in accordance with one aspect of the present invention.

FIG. 9 is a schematic representation of the transfer of electrons and protons in an anode compartment of an analyte sensor in one embodiment of the present invention.

### DETAILED DESCRIPTION

Generally, an analyte sensor is, or is part of a device for detecting at least one chemical or biological agent. Such a device is adapted to receive a sample upon which it acts to provide a response that indicates the presence of the agent and/or the amount of such agent. Alternatively, the device or sensor can be placed into the sample for which detection of an agent is desired. Furthermore, a sensor or device can comprise at least one sensor but can also comprise more than one, or an array of sensors, in order to identify or measure more than one agent.

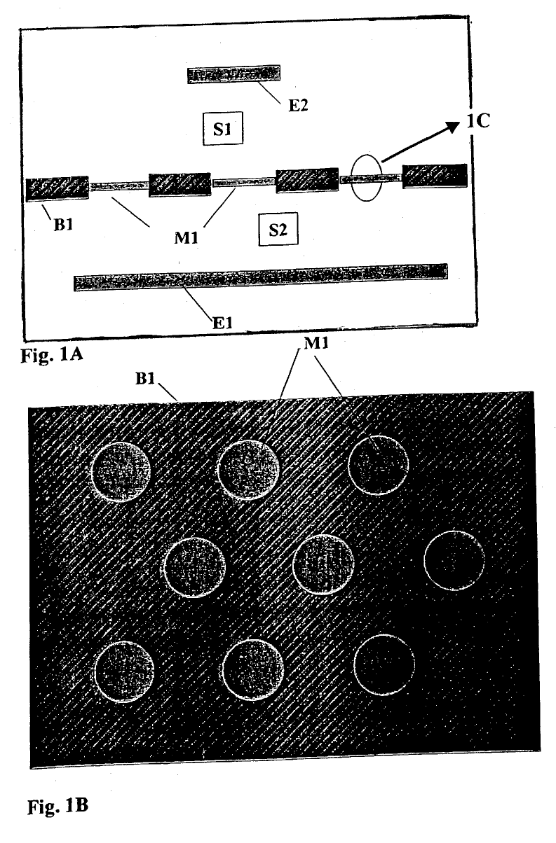

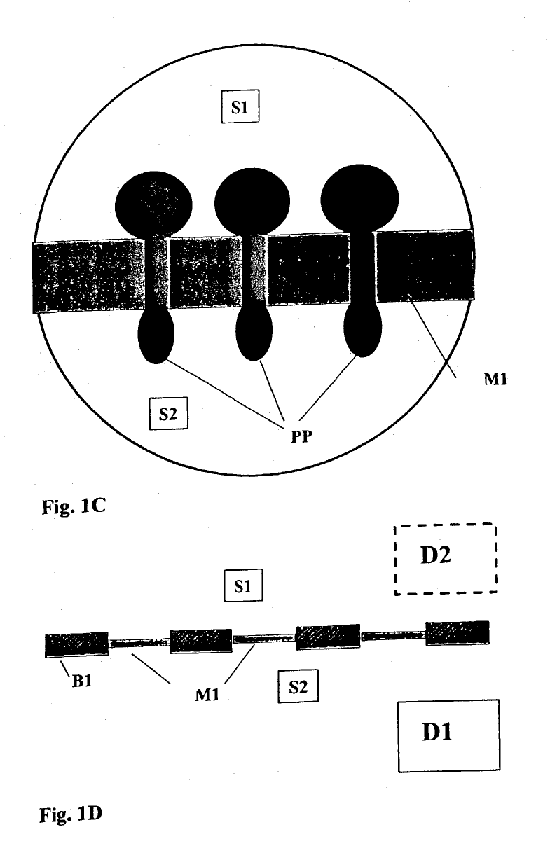

FIGS. 1A-C illustrate one embodiment of an analyte sensor having a detector which is based upon detection of electrical current. The analyte detector comprises a first electrode E1, a second electrode E2, and a barrier B1, the barrier B1 having openings across which are disposed membranes M1. In one aspect, the membrane M1 is a polymer-containing biocompatible membrane, a proton-tunneling membrane (“PTM”) or combination thereof. The barrier B1 and membranes M1 separate a first compartment or side S1 and a second compartment or side S2. Second electrode E2 provides one manner of providing a counter electrode for electrode E1.

The membranes M1 may or may not incorporate one or more polypeptides or polypeptide complexes PP which are capable of participating in the transport of a species across the membrane from one compartment to the other. FIG. 1C is a close-up of that portion of membrane M1 designated in FIG. 1A. Transmembrane polypeptides PP are depicted in association with membrane 1 spanning the entirety of the membrane, having portions exposed on both sides S1 and S2. This construction is referred to as a transmembrane polypeptide. It will be recognized that, while schematic polypeptides PP are shown as transmembrane polypeptide, other membrane associations are also contemplated by the present invention. Integral polypeptides which are embedded in or associated with the membrane, but which may or may not be transmembrane polypeptides, are also contemplated. Peripheral polypeptides having association with various structures on the surface of the membrane or with integral proteins in the formation of polypeptide complexes are also contemplated. In one preferred embodiment there are created membranes M1 having, primarily at side S1, the surface of the polypeptide that will interact with the analyte or analyte derivative-that is, having the active site of the polypeptide disposed so as to interact with the analyte or analyte derivative on side S1.

If a polypeptide is used as part of the analyte chemistry or in association with a membrane, the polypeptide, which may include a polypeptide complex, may interact with the analyte directly, or it may interact with a “derivative of the analyte”. “Derivative of the analyte” refers to any species formed in the first compartment upon or following introduction of the analyte, including, for example, a proton or reaction product or by-product, and includes those species which act as a catalyst and are not consumed, or are merely a reactant that allow the detected species to be generated. In the case where a derivative of the analyte interacts with the polypeptide, rather than or in addition to the analyte itself, the first compartment further comprises the component(s) necessary to effect formation of the derivative of the analyte when the analyte is present.

Similarly, the detector may not necessarily interact directly with the species transported across the membrane in response to introduction of the analyte, but may act upon a “derivative of the transported species.” For purposes of the present invention, a derivative of the transported species means any species created in response to the presence of the transported species, such as a proton, cation or any positively charged species; an electron, anion or any negatively charged species; or a compound; and further including the product of a reaction where the transported species acts as a catalyst and is not consumed. In the case where a derivative of the transported species, rather than or in addition to the transported species itself, the first or second compartment, as appropriate, comprises the necessary component(s) to effect formation of the detected derivative when the transported species is present.

The term “transport”, as used herein, generally refers to the movement of a species, be it protons, electrons, atoms or molecules, across a membrane, and can refer to both active and passive processes. In a preferred aspect, the analyte sensor includes a biocompatible membrane wherein an associated polypeptide is capable of participating in the transport of molecules, atoms, protons or electrons from a first compartment to a second compartment across the membrane, which includes participating in the formation of molecular structures that facilitate such transport. In a more particularly preferred aspect of the invention, the polypeptide is a redox enzyme and/or is an enzyme capable of participating in the transmembrane transport of protons.

It is not necessary that the species remain unchanged as it is transported across a barrier or membrane; a chemical reaction, chain or series of reactions may take place within the membrane which results in the transfer of a species, altered or unaltered, across a membrane. That is, while reference is made to a “transported species”, this also encompasses the situation where the species that enters the membrane at a first side is not the same species that is introduced into the opposite compartment. When discussing transporting protons across a biocompatible membrane, PTM, or other barrier, it will be appreciated that neither the exact mechanism, nor the exact species transferred need be known. In the case of “protons”, the transferred species might be a proton per se, a positively charged hydrogen, a hydronium ion, H₃O⁺ or indeed some other positively charged species. For convenience, however, these are collectively characterized herein as “protons.” The transported species can also include negatively charged particles, and can include both anions and cations.

“Biocompatible membrane” as used herein is one or more layers of a synthetic polymeric material forming a sheet, plug or other structure suitable for use as a membrane and is associated with a polypeptide or other molecule, often of biological origin. By “biocompatible,” it is meant that the membrane comprises a synthetic polymer material that will not incapacitate or otherwise block all of the functionality of a polypeptide suitable for use with the present invention when the membrane and polypeptide are associated with one another. A biocompatible membrane may also be a PTM. Biocompatible membranes are discussed in detail in the commonly assigned co-pending U.S. application Ser. No. 10/213,530 entitled “Biocompatible Membranes and Fuel Cells Produced Therewith”, which is a continuation-in-part of International Application No. PCT/US02/11719 filed Apr. 15, 2002; and each of U.S. applications Ser. Nos. 10/123,022, 10/123,039, 10/123,021, 10/123,020 and 10/123,008, all of which were filed Apr. 15, 2002, the disclosures of which are hereby incorporated by reference.

The terms “barrier” and “membrane” as used herein both refer to a structure such as a sheet, layer or plug of a material that may be used to selectively segregate space, fluids (liquids or gases), solids and the like. The term “barrier” however, often refers to a larger structure, of which membranes may be only a portion, often the barrier containing holes or perforations across which a membrane is positioned, the barrier often operating as a support for a membrane. “Membranes” may include biological, biocompatible, and proton-tunneling membranes. Both “barriers” and “membranes” as used herein may include semi-permeable materials that allow the passage or diffusion of some species from one compartment to the other, while being impermeable to other species. Both barriers and membranes as referred to herein may be designed to exhibit “proton-tunneling” activity.

In some aspects of the present invention, a semi-permeable barrier is employed to separate a first compartment adapted for introduction of a sample potentially containing the targeted analyte, and a second compartment. Components disposed in the first compartment, which may or may not be embedded or immobilized in relation to any of the walls defining the compartment are adapted to react with the analyte to produce a species, be it a proton, electron, atom or molecule, which can then travel across the barrier. The reaction may cause a species to be produced which because of its size, shape or charge, or a combination thereof, are capable of being transported across the barrier. “Transport” herein being used to refer to the situation where such species travel as a result of a concentration gradient.

In yet another aspect, the barrier may refer to a relatively proton impermeable structure separating a first and second compartment, whereby the interaction of the analyte with components in a first compartment, which may include an enzyme, causes the production of protons which are capable of being transported across the barrier via proton-tunneling.

Preferably, the barrier B1 is not permeable to the analyte or derivative thereof to be acted upon by the polypeptide PP. However, some leakage is acceptable, so long as the needed signal to noise ratio for a given measurement is achieved.

“Associated” in accordance with the present invention can mean a number of things depending on the circumstances. A polypeptide can be associated with a biocompatible membrane or a PTM by being bound to one or more of the surfaces thereof, and/or by being wedged or bound within one or more of the surfaces of the membrane (such as in recesses or pores). Reference to being “bound” includes physical binding as well as electrostatic hydrogen bonding and ionic or covalent bonds, or a combination thereof. The “associated” polypeptide can be disposed within the interior of the membrane or in a vesicle or lumen contained within the membrane. Polypeptides can also be disposed between successive layers. Polypeptides may be embedded in the membrane as well. Indeed, in a particularly preferred embodiment, the polypeptide is embedded or integrated in the membrane in such a way that it is at least partially exposed through at least one surface of the membrane and/or can participate in a redox reaction or participate in either the polypeptide mediated transporting of a molecule, atom, proton or electron, or proton-tunneling as a method of moving protons, from one side of the membrane to the other.

The terms “participate” and “participating”, in the context of transporting a molecule, atom, proton or electron, from one side of a barrier to another includes active transport where, for example, a polypeptide physically or chemically “pumps” the molecule, atom, proton or electron across a barrier, including situations where pumping is with or against a pH, concentration or charge gradient or any other active transport mechanism. However, “participation” need not be so limited. For example, a polypeptide can participate in transport by forming structures which facilitate such transport. Further, a polypeptide may participate in transport by participating in a chemical reaction whereby the product of such reaction is rendered capable, with or without further modification, of being transported across a barrier, either actively or passively.

“Polypeptide mediated transport” includes those processes in which a barrier associated polypeptide plays a role (excluding passive diffusion) in the transport of a species across a barrier, in ways other than merely structurally providing a static channel. Stated another way, “polypeptide mediated transport” means that the presence of the barrier associated polypeptide results in effective transport of a species from one side of the membrane to another in response to something other than concentration alone. “Participate,” in the context of a redox reaction, means that the polypeptide causes or facilitates the oxidation and/or reduction of a species, or conveys to or from that reaction protons, electrons or oxidized or reduced species. In the context of a PTM, “participate” and “participating” means playing a role in the transport of protons across a PTM. Without wishing to be bound by any particular theory of operation, this could include the transfer of protons by proton-tunneling across a layer or membrane. This term excludes mere proton permeability. Again, without wishing to be bound by any particular theory of operation, polypeptides, if present, may facilitate the entry of protons into the surface of a PTM and, thereafter, proton-tunneling may complete the transport of the proton to the other side of the biocompatible membrane or PTM.

“Polypeptide(s)” includes at least one molecule comprising at least four amino acids that is capable of participating in a chemical reaction, often, but not necessarily, as a catalyst, or participating in the transporting of a molecule, atom, proton or electron from one side of a barrier (including a membrane, biocompatible membrane, PTM, or combination thereof) to another, or participating in the formation of molecular structures or compounds that facilitate or enable such reactions or transport. The polypeptide can be single stranded or multiple stranded, and can exist in a single subunit or multiple subunits. It can be made up of exclusively amino acids or combinations of amino acids and other chemical compounds or molecules. This can include, for example, pegalated peptides, peptide nucleic acids, peptide mimetics, and neucleoprotein complexes. Strands of amino acids that include such modifications as the product of, for example, glycosolation are also contemplated. Polypeptides in accordance with the present invention are generally biological molecules or derivatives or conjugates of biological molecules. Polypeptides can therefore include molecules that can be isolated, as well as molecules that can be produced by recombinant technology or which must be, in whole or in part, chemically synthesized. The term therefore encompasses naturally occurring proteins and enzymes, mutants of same, derivatives and conjugates of same, as well as wholly synthetic amino acid sequences and derivatives and conjugates thereof. In one embodiment, polypeptides in accordance with the present invention can participate in the transport of molecules, atoms, protons and/or electrons from one side of a barrier or membrane to another side thereof, can participate in oxidation or reduction, or are charge driven proton pumping polypeptides such as DH⁻ Complex I (also referred to as “Complex 1”).

“Bioactive agents” include a substance such as a chemical that can act on a cell, virus, tissue, organ or organism, including, but not limited to insecticides, drugs, or toxic agents, to create a change in the functioning of the cell, virus, organ or organism. Because a preferred analyte sensor of the present invention includes a membrane which incorporates at least partially functional polypeptides which may be found in some form in a living organism, the analyte sensors are particularly suited for identifying “bioactive agents”.

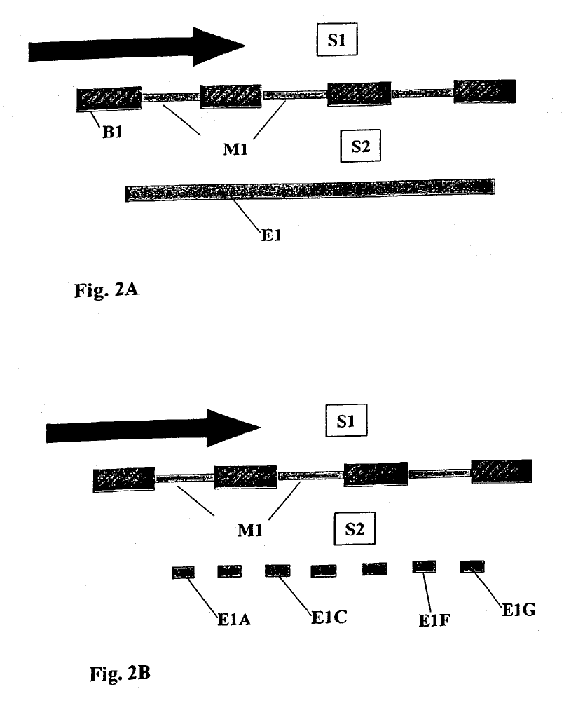

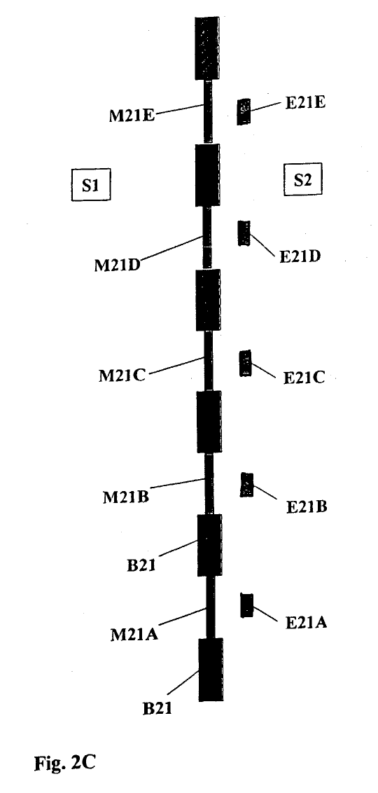

FIGS. 2A-B illustrate an analyte sensor or analytical cell in use. A solution containing the targeted analyte is introduced into side Si as illustrated by the arrow. The PP acts on the analyte or derivative (when present), creating a change measurable at side S2. In a preferred embodiment, the measurable change creates a difference in electrical potential at electrode E1, as measured against a standard electrode E2 disposed on side S1.

As illustrated in FIG. 2B, the electrode can be divided into multiple electrodes (first electrode E1A, second electrode E1B, and so on through, in the illustration, seventh electrode E1G). The separate electrodes can be used to provide temporal control data. For example, if the analytical cell is operating correctly and the analyte is introduced from the left side as illustrated, then the effect generated by the polypeptide (PP) should begin at the first electrode and proceed to the succeeding electrodes with appropriate relative kinetics. Temporal control data can be generated, for example, in two ways, providing two sets of data which can be used individually or together to analyze the sample introduced. In a first method, forced flow is used so that all of the analyte as well as any carrier solvent are moving at the same rate across the compartment. The output of any particular detector in the series will be affected by how much of the targeted analyte has been depleted in connection with interactions that result in the transport of a species across the membranes earlier in the sequence. A second method involves free flow, in which the rate of response to an individual analyte in a mixture will differ depending on mobility of the analyte.

As illustrated in FIG. 2C, separate electrodes (E21A-E21E) can be aligned with separate membranes (M21A-M21E). In this embodiment, the electrodes can be situated to be effectively insensitive to events across the non-aligned membranes. Thus, the separate membranes can each contain a distinct polypeptide or mix of polypeptides, and the same sample can be passed through side Si to generate different responses capable of producing separate analytical results. For example, mixtures of polypeptides can be used in combinatorial screening to identify sources of activity, or, conversely, in screenings where an analyte (or derivative thereof) does not induce activity in any of several separate families of polypeptides. Where separate membranes otherwise not compartmentalized can be separately used for detection, the device (or component of a larger array) can be termed a “combined array of analyte sensors”.

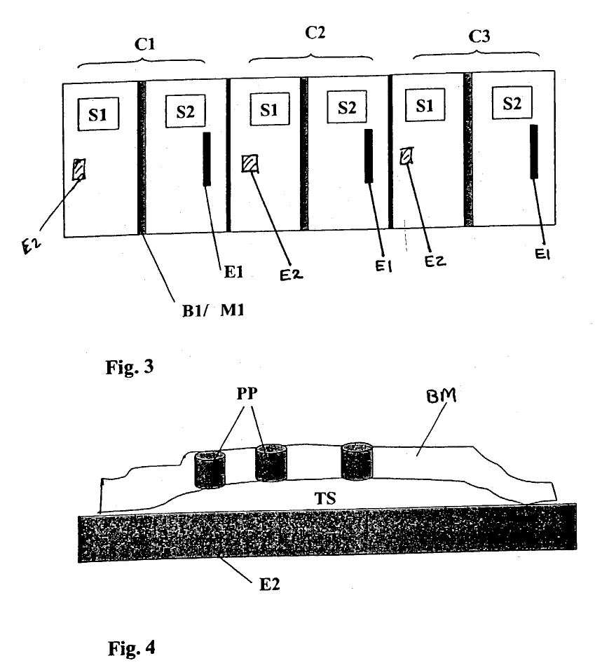

In another embodiment, the cell or analyte sensor can be part of an array of such cells or sensors, each of which can be adapted to detect or measure different analytes, or different samples with respect to the same analyte. Such an array is schematically illustrated in FIG. 3, with first cell C1, second cell C2, and so forth.

In yet another embodiment, the compartments of an array can be cascaded, that is, where the second compartment of a first analyte sensor is the first compartment of a second analyte sensor for further analysis of the sample. In this embodiment, the transported species of the first analyte sensor is effectively the analyte of the second sensor. For example, where several analytes are known to react with respect to the components or polypeptides of the first sensor to produce distinct transported species, the second and/or subsequent sensor in the cascade can be adapted to distinguish between the distinct transported species of the first sensor, thereby detecting the analytes. In yet another aspect, where electrical charge is measured by the detector, the cathode compartment of the first analyte sensor can serve as the anode compartment of a second analyte sensor. The analyte, or a derivative of the analyte, moves across the first compartment to participate in interactions that create the first detection event and the second detection event involves the second membrane in the series. Of course, there is no limit to the number of barriers, membranes, sensors and detection events in the cascade; preferably, there are at least two detection events. In another embodiment of a cascading sensor array, upon testing of a sample in a first analyte sensor, a sample is taken from a compartment of a first analyte sensor and applied to a second, analyte sensor. A transfer element, such as tubing and a pump may be employed for this purpose. The transfer may be automated or manual.

In a preferred aspect, where electrical current is detected, one electrode is disposed in a first compartment, and a second electrode is disposed in the second compartment. This is illustrated by electrodes E1 and E2 in the various figures, which detect a chemical change caused by the polypeptide interacting with an analyte or a derivative thereof.

Of course, many other methods of detection known to those in the art may be employed. Such methods can include, without limitation, colorimetric (as developed chemically, enzymatically, immunologically or the like), chromatographic, spectroscopic (including through the use of mass spectroscopy), and any other methods of detecting a chemical substance. Detectors can include, for example, current or voltage meters; gas chromatographs; liquid chromatographs; mass spectrometers; nuclear magnetic resonance analyzers, infra-red, ultraviolet and/or Raman spectrophotometers, C,H,N,O detectors, moisture detectors, conductivity sensors, thermometers, oxygen sensors, pH detectors, colorimetric detectors, turbidity meters, particle counters, particle size detectors and the like.

A detector useful in the present invention may be disposed in either the first compartment or the second compartment, depending upon the direction of transport and the detection method employed. And, in some instances, more than one detection method can be employed, and indeed may be necessary to identify a specific analyte. In yet other aspects, a sample generated during or after introduction of an unknown sample into the sensor or cell of the present invention is removed or directed from the second or first compartment and supplied to an external detector.

For any detector, the particular substance detected may or may not be the species transported across the barrier; it may be a further surrogate generated for example by reagents known in the art and provided at side S2. In some aspects, the species transported across la barrier as a result of interaction with the polypeptide (PP), or as a result of a chemical reaction, on introduction of the analyte, can be measured, where appropriate, by use of an oxidative derivative of such molecular species. For example, glucose oxidase produces electrons from glucose on converting glucose to gluconic acid, which electrons can be measured with an electrode.

An analytical cell utilizing a detector D1 in place of the preferred electrodes is shown in FIG. 1D. Where secondary sampling or separations are utilized in detection, as in chromatography or mass spectrometry, mechanized transfer of sample from side S2 to the separation device, as known in the art, is preferably used. If a derivative is detected, further components, such as enzymes or reagents, needed to generate the measured substance based on the presence of the transported species will be provided in the appropriate compartment of the analyte sensor.

Many of the polypeptides (or complexes) proposed for use in the analytical cell or sensor of the invention are the same as those that have been used in systems in which an analytical catalyst polypeptide is incorporated into a biological membrane affixed to an electrode. As used in such a system, it is believed that the biological membrane forms pockets of solution adjacent to the electrode and entrapped by the membrane, as illustrated in FIG. 4. In FIG. 4, a biological membrane (BM) is adhered to electrode E2, and has incorporated polypeptide, PP. Analytically significant events are believed to occur in trapped solution TS. While the illustration in FIG. 4 shows a relatively large single volume of trapped solution, the solution can comprise numerous very small volumes, or very shallow volume. Such small volumes occur, for example, where the biological membrane is associated with the electrode with thio-containing linkers, as taught for example in WO93/21528 (Eur. Inst. Technol.).

In one embodiment, the analyte sensor can be flushed of any solution subjected to analysis and re-used. Where detection occurs at side S2, that, side is preferably also flushed between uses, using if appropriate, a separate flush or setup solution. Flushing can be effected with a first solution adapted to be biocompatible with the polypeptide, followed by a reintroduction of a solution adapted to support the analytical reactions. In particular, where the cost of a supportive solution is reasonable, the flush can comprise such reactive supportive solution.

In an alternative embodiment, the analyte sensor is disposable, this is particularly useful for sensors adapted to detect toxic or hazardous substances. Disposable sensors may be particularly useful in medical diagnostics for detecting analytes in a sample from a patient. Such analytes include, but are not limited to, amino acids, enzyme substrates or products indicating a disease state or condition, other markers of disease states or conditions, drugs of abuse, therapeutic and/or pharmacologic agents, electrolytes, physiological analytes of interest (e.g., calcium, potassium, sodium, chloride, bicarbonate (CO₂), glucose, urea (blood urea nitrogen), lactate, hematocrit, and hemoglobin), lipids, and the like. In another embodiment, the analyte sensor may be used for monitoring blood glucose. Furthermore, a disposable sensor may be particularly useful in connection with detection of radioactive species.

The Barrier As A Support

In a preferred embodiment in accordance with the present invention, a membrane may be disposed and/or formed within or across apertures or perforations of a barrier or support, designated in the figures as B1. A “perforated barrier” is one that has at least one hole, aperture or pore into which, or over which, a membrane can be disposed. The perforations may be formed, for example, by punching, drilling, laser drilling, stretching, and the like.

In certain preferred embodiments, the barrier is glass or a polymer (such as polyvinyl acetate, polydimethylsiloxane (PDMS), Kapton® (polyimide film, Dupont de Nemours, Wilmington, Del.), a perfluorinated polymer (such as Teflon®, from DuPont de Nemours, Wilmington, Del.), polyvinylidene fluoride (PVDF, e.g., a semi-crystalline polymer containing approximately 59% fluorine sold as Kynar™ by Atofina, Philadelphia, Pa.), PEEK (defined below), polyester, UHMWPE (described below), polypropylene or polysulfone), soda lime glass or borosilicate glass, or any of the foregoing coated with metal. The metal can be used to anchor a biocompatible membrane or a PTM (such as a monolayer or bilayer of amphiphilic molecules) (e.g., with thiol linkers). In a particularly preferred aspect of the present invention, the perforated barrier is made of a dielectric material. In one embodiment, the perforated or porous substrate is a film. Supports or substrates with high natural surface charge densities, such as Kapton and Teflon, are in some embodiments preferred. Where non-covalent interactions support the membrane's attachment, then the membrane-interacting portions of the support can directly be, or be coated or derivatized to provide, a hydrophobic surface that stabilizes an interaction with the membrane.

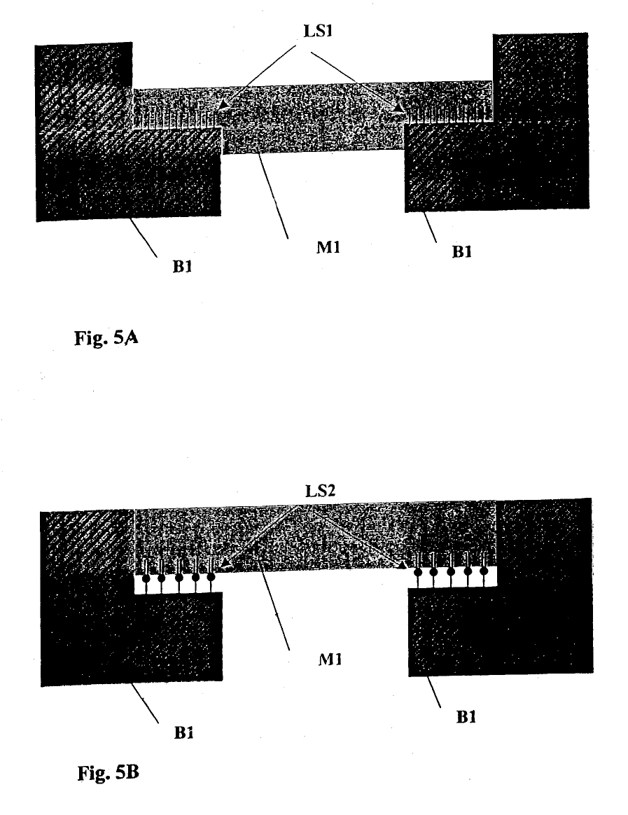

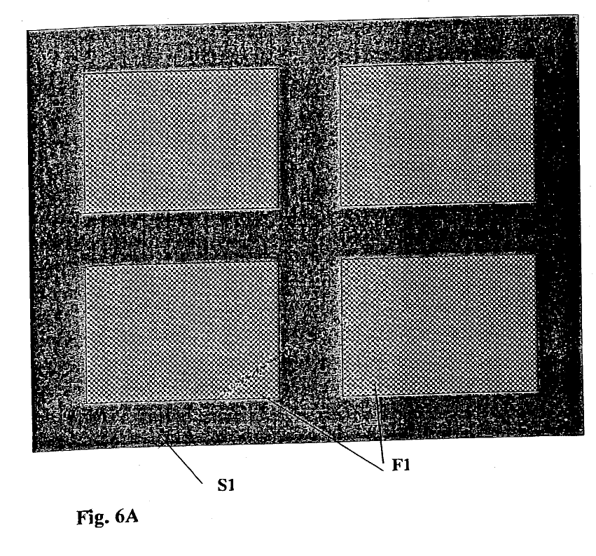

While anchoring the membrane is not essential, as has been established with membranes with incorporated Complex I, anchoring techniques known in the art can be used to stabilize the membrane. For example, as illustrated in FIG. 5A, an alkylene tail can be attached to an appropriate surface to form lipophilic surface LS1. Surface attachments can use any number of chemistries known in the art, including thiol mediated linkages with a gold coated surface. Or, as illustrated in FIG. 5B, a phospholipid can be attached via a polyethylene oxide bridge from the phospholipid head group to a surface attachment (see, e.g., WO 93/21528).

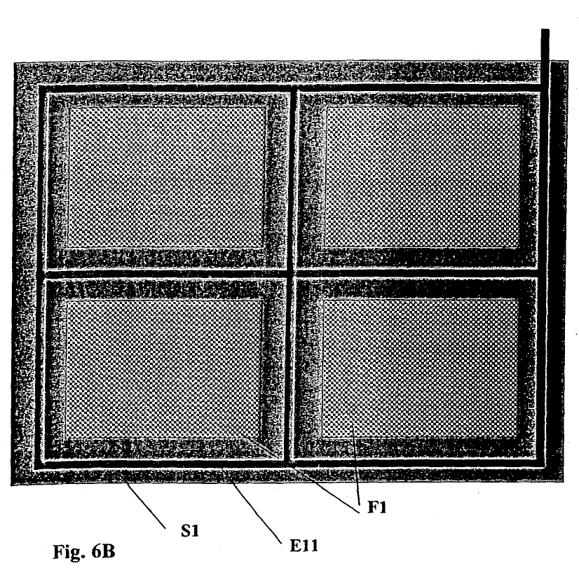

The barrier can further be fabric or mesh of an appropriate material. Such fabric provides large surface areas, while nonetheless allowing the membrane to be supported and stabilized at regular, closely spaced intervals. Such fabric support can be further supported by a scaffold of stronger material. For example, scaffold S1, illustrated in FIG. 6A, supports fabric F1.

Such fabric or mesh, when formed of an appropriate polymer, can be compressed while heated to partially or fully merge the overlapping fabric strands and provide the fabric or mesh with a smoother surface.

As illustrated in FIG. 6B, the electrode can be incorporated in the barrier, while nonetheless not supporting or attaching to the membrane, such as in the way electrode Ell is situated.

Where a perforated but otherwise solid barrier is used, the thickness of the barrier is, for example, from 15 micrometers (μm) to 50 μm, or from 15 μm to 30 μm. The width of the perforations is, for example, from 20 μm to 200 μm, or 60 μm to 140 μm, or 80 μm to 120 μm.

Perforations in the barrier and metallized surfaces can be constructed, for example, with masking and etching techniques of photolithography well known in the art. Alternatively, the metallized surfaces (electrodes can be formed, for example, by (i) thin film deposition through a mask, (ii) applying a blanket coat of metallization by thin film photo-defining, selectively etching pattern into the metallization, or (iii) photo-defining the metallization pattern directly without etching using metal impregnated resist (DuPont Fodel process, Drozdyk et al., “Photopatternable Conductor Tapes for PDP Applications,” Society for Information Display 1999 Digest, 1044-1047; Nebe et al., U.S. Pat. No. 5,049,480). In one embodiment, the barrier is a film. For example, the barrier can be a porous film that is rendered non-permeable outside the “perforations” by the metallizations. The surfaces of the metal layers can be modified with other metals, for instance by electroplating. Such electroplatings are, for example, with chromium, gold, silver, platinum, palladium, nickel, mixtures thereof, or the like, preferably gold and platinum.

In an alternative embodiment, the barrier is not associated with a membrane at all, but made of a synthetic material which exhibits proton-tunneling activity. In this instance, protons released into a first compartment as a result of introduction of the analyte into the first compartment, and interaction of the analyte or a derivative thereof with a polypeptide or other reactive component disposed in the first chamber, are transported across the barrier, the transported species, or a derivative thereof, being detected, indicating the presence of the analyte.

Membrane Formation, Protein Incorporation

A biological membrane can be formed across the perforations or openings in a fabric barrier and polypeptide incorporated therein by, for example, the methods described in detail in Niki et al., U.S. Pat. 4,541,908 (annealing cytochrome C to an electrode) and Persson et al., J. Electroanalytical Chem. 292: 115, 1990. Such methods can comprise the steps of: making an appropriate solution of lipid or other amphilphiles and polypeptide, where the polypeptide may be supplied to the mixture in a solution stabilized with a detergent; and, once an appropriate solution of lipid or other amphiphiles and polypeptide is made, the perforated dielectric substrate is dipped into the solution to form the polypeptide-containing membrane layers. Sonication or detergent dilution can facilitate polypeptide incorporation into a layer. See, for example, Singer, Biochemical Pharmacology 31: 527-534, 1982; Madden, “Current concepts in membrane protein reconsitution,” Chem. Phys. Lipids 40: 207-222, 1986; Montal et al., “Functional reassembly of membrane proteins in planar lipid bilayers,” Quart. Rev. Biophys. 14: 1-79, 1981; Helenius et al., “Asymmetric and symmetric membrane reconstitution by detergent elimination,” Eur. J. Biochem. 116:27-31, 1981; Volumes on membranes (e.g., Fleischer and Packer (eds.)) In Methods in Enzymology series, Academic Press.

One method of incorporating a polypeptide into a biological membrane is as follows: Incorporation of the polypeptide (e.g., the proton transporting enzyme Complex I) is accomplished by fusion with the membrane, in a solution containing 10 mM calcium chloride, of vesicles that contained the polypeptide. Use of calcium as an agent to promote the fusion of vesicles with membranes is well recognized in the art, as illustrated by: Landry et al., “Purification and Reconstituion of Epithelial Chloride Channels,” 191 Methods in Enzymology 572, 582 (1990) (at 582); Schindler, “Planar Lipid-Protein Membranes . . . ,” 171 Methods in Enzymology 225, 226 (1989). More specifically, the vesicles are injected onto the membrane, then incubated on side Si in a relatively small volume, such as 500 microliter. This is essentially the method of Landry et al. (at 582), or Schindler (at 236). The protein-containing vesicles are prepared by incubating a detergent solution of the protein with vesicles that had been freshly formed from lipids using sonication. This is essentially the method described in Schindler at 252 (which uses vortexing instead of sonication). This method has been successfully applied to incorporate Complex I as obtained from over-expressing E. coli into a stable membrane formed across a perforation in a Teflon barrier.

It will be recognized that the method of incorporating a particular polypeptide into a particular membrane will generally be optimized by ordinary experimentation. Further guidance is provided from the many membrane proteins that have now been successfully incorporated into a biological membrane material.

Methods of forming membranes tend to share a commonality. A thin partition made of a hydrophobic material such as Teflon with a small aperture has a small amount of lipid (or other amphiphile) introduced. The amphiphile-coated aperture is immersed in a dilute electrolyte solution upon which the lipid droplet will thin and spontaneously self-orient into a planar bilayer spanning the aperture. Membranes of substantial area have been prepared using this general technique. Common methods well known in the art for formation of the membranes themselves are the Langmuir-Blodgett technique, self-assembly technique, and injection technique. These are described in detail in copending U.S. application Ser. No. 10/213,530, the disclosure of which is incorporated herein by reference.

Membrane layers can be formed against a solid material, such as by coating onto glass, carbon that is surface modified to increase hydrophobicity, or a polymer such as polydimethylsiloxane (PDMS). Polymers such as PDMS provide an excellent porous support on which membranes can be used to span the pores.

Coating methods (which are particularly preferred with block copolymers) include a first coating or lamination of conductor (such as copper cladding), followed by plating, sputtering or using another coating procedure to coat with a noble conductor such as gold or platinum. Another method is directly sputtering an attachment layer, such as chromium or titanium onto the support, followed by plating, sputtering or other coating procedure to attach a noble conductor. The outer metal layer is favorably treated to increase its hydrophobicity, such as with dodecane-thiol. Supports with high natural surface charge densities, such as Kapton and Teflon, are in some embodiments preferred.

In some embodiments, the polypeptide is not anticipated to have trans-membrane effect, or such trans-membrane effect is facilitated by an lipophilic electron transfer mediator. For purposes of the present invention, trans-membrane effect means that the polypeptide itself has the capability of fully transporting the species across the membrane. In these cases, membrane association can be effected by linkers (typically hydrophilic) known in the art for connecting the polypeptide to the polar end of an amphipathic compound that incorporates into the membrane. The phospholipid derivatives with a polyethylene oxide linker described in WO93/21528 (Eur. Inst. Technol.) are illustrative. In cases where there is no trans-membrane effect, the analyte sensor provides an environment for the polypeptide that supports re-use of the sensor with reduced interference from time dependent adsorption of the polypeptide to a polymer or glass support. Also in such cases, side S2 can be of very limited size.

Biocompatible Membranes

Biocompatible membranes useful in an analyte sensor of the present invention can be formed from any synthetic polymer material that, when associated with one or more polypeptides as described herein, meet the objectives of the present invention. Many of the same methods and materials used to produce such membranes also apply to the formation of PTMs, which are discussed in more detail herein below.

Useful synthetic polymer materials include polymers, copolymers and block copolymers and mixtures of same. These can be bound, crosslinked, functionalized or otherwise associated with one another. “Functionalized” means that the polymers, copolymers and/or block copolymers have been modified with end or reactive groups that are selected to perform a specific function, whether that be polymerization, (crosslinking of blocks, for example), anchoring to a particular surface chemistry (use of, for example, certain sulfur linkages), facilitated electron transport via covalently linking an electron carrier or electron transfer mediator, and the like known to the art. Typically, these end or reactive groups are not considered a constituent of the polymer or block itself and are often added at the end of or after synthesis. Synthetic polymer materials are generally present on the finished membrane (the membrane in condition for use) in an amount of at least about 50% by weight of the finished membrane, more typically at least about 60% by weight of the finished membrane and often between about 70 and about as much as 99% by weight thereof. A portion of the total amount of the synthetic polymer material may be a stabilizing polymer, generally up to about a third, by weight based on the weight of the total synthetic polymer material in the finished biocompatible membrane.

Biocompatible membranes useful in the present invention are preferably produced from one or more block copolymers such as A-B, A-B-A or A-B-C block copolymers, with or without other synthetic polymer materials such as polymers or copolymers, and with or without additives.

One suitable block copolymer is described in a series of articles by Corinne Nardin, Wolfgang Meier and others. Angew Chem Int. Ed. 39: 4599-4602, 2000; Langmuir 16: 1035-1041, 2000; Langmuir 16: 7708-7712, 2000. It is characterized as a functionalized poly(2-methyloxazoline)-block-poly (dimethylsiloxane) -block-poly(2-methyloxazoline) triblock copolymer.

The Nardin-Meier polymer can provide relatively large membranes that can incorporate functional polypeptides. The methacrylate moieties at the ends of the polymer molecules allow for free-radical mediated crosslinking after incorporating polypeptide in order to provide greater mechanical stability.

Where such synthetic membranes are used with polypeptides that prefer or require the presence of certain lipids, such lipids are for example provided in the process of inserting the polypeptide into the membrane, or incorporated into the membrane formation.

Other examples of useful block copolymers are listed in copending U.S. application Ser. No. 10/213,530, the disclosure of which is incorporated herein by reference, and include, without limitation: Amphiphilic block copolymers; Triblock copolyampholytes from 5-(N,N-dimethylamino)isoprene, styrene, and methacrylic acid; Styrene-ethylene/butylene-styrene triblock copolymers, sold under the tradename KRATON® available from the Shell Chemical Company. The preferred block copolymers are of the styrene-ethylene/propylene (S-EP) types and are commercially available under the tradenames KRATON®, also available from the Shell Chemical Company; Siloxane triblock copolymers; and PDMS-b-PCPMS-b-PDMSs (PDMS=polydimethylsiloxane, PCPMS=poly(3-cyanopropylmethyl-cyclosiloxane). DEO-CPPO-CPEO triblock copolymer; PEO-PDMS-PEO triblock copolymer [Polyethylene oxide (PEO) is soluble in the aqueous phase, while the poly-dimethyl siloxane (PDMS) is soluble in oil phase]; PLA-PEG-PLA triblock copolymer; Poly(styrene-b-butadiene-b-styrene) triblock copolymer [Commonly used thermoplastic elastomers, includes Styrolux from BASF, Ludwigshafen, Germany]; Poly(ethylene oxide)/poly(propylene oxide) triblock copolymer films [Pluronic F127, Pluronic P105, or Pluronic L44 from BASF, Ludwigshafen, Germany]; Poly(ethylene glycol)-poly(propylene glycol) triblock copolymer; PDMS-PCPMS-PDMS (polydimethylsiloxane-polycyanopropylmethylsiloxane) triblock copolymer; Azo-functional styrene-butadiene-HEMA triblock copolymer, Amphiphilic triblock copolymer carrying polymerizable end groups; Syndiotactic polymethylmethacrylate (sPMMA)-polybutadiene (PBD)-sPMMA triblock copolymer, Tertiary amine methacrylate triblock (AB diblock copolymer); Biodegradable PLGA-b-PEO-b-PLGA triblock copolymer; Polylactide-b-polyisoprene-b-polylactide triblock copolymer; PEO-PPO-PEO triblock copolymer; Poly(isoprene-block-styrene-block-dimethylsiloxane) triblock copolymer; Poly(ethylene oxide)-block-polystyrene-block-poly(ethylene oxide) triblock copolymer; Poly(ethylene oxide)-poly(THF)-poly(ethylene oxide) triblock copolymer; Ethylene oxide triblock; Poly E-caprolactone [Birmingham Polymers]; Poly(DL-lactide-co-glycolide) [Birmingham Polymers]; Poly(DL-lactide) [Birmingham Polymers]; Poly(L-lactide) [Birmingham Polymers], Poly(glycolide) [Birmingham Polymers]; Poly(DL-lactide-co-caprolactone) [Birmingham Polymers]; Styrene-Isoprene-styrene triblock copolymer [Japan Synthetic Rubber Co.]; PEO/PPO triblock copolymer; PMMA-b-PIB-b-PMMA [linear triblock TPE]; PLGA-block-PEO-block-PLGA triblock copolymer [Sulfonated styrene/ethylene-butylene/styrene (S-SEBS) TBC polymer proton conducting membrane. Available as Protolyte A700 from Dais Analytic, Odessa Fla.]; Poly(1-lactide)-block-poly(ethylene oxide)-block-poly(l-lactide) triblock copolymer; Poly-ester-ester-ester triblock copolymer; PLA/PEO/PLA triblock copolymer; PCC/PEO/PCC triblock copolymer [the above polymers can be used in mixtures of two or more. For example, in two polymer mixtures measured in weight percent of the first polymer, such mixtures can comprise 20-25%, 25-30%, 30-35%, 35-40%, 40-45% or 45-50%]; various block copolymers available from Polymer Source, Inc., Dorval, Quebec, Canada, including: Poly(t-butyl acrylate-b-methyl methacrylate-b-t-butyl acrylate); Poly(t-butyl acrylate-b-styrene-b-t-butyl acrylate) [Polymer Source, Inc.]; Poly(t-butyl methacrylate-b-t-butyl acrylate-b-t-butyl methacrylate); Poly(t-butyl methacrylate-b-methyl methacrylate-b-t-butyl methacrylate); Poly(t-butyl methacrylate-b-styrene-b-t-butyl methacrylate); Poly(methyl methacrylate-b-butadiene(1,4 addition)-b-methyl methacrylate); Poly(methyl methacrylate-b-n-butyl acrylate-b-methyl methacrylate); Poly(methyl methacrylate-b-t-butyl acrylate-b-methyl methacrylate); Poly(methyl methacrylate-b-t-butyl methacrylate-b-methyl methacrylate); Poly(methyl methacrylate-b-dimethylsiloxane-b-methyl methacrylate); Poly(methyl methacrylate-b-styrene-b-methyl methacrylate); Poly(methyl methacrylate-b-2-vinyl pyridine-b-methyl methacrylate); Poly(butadiene(1,2addition)-b-styrene-b-butadiene(1,2addition)); Poly(butadiene(1,4addition)-b-styrene-b-butadiene(1,4addition)); Poly(ethylene oxide-b-propylene oxide-b-ethylene oxide); Poly(ethylene oxide-b′-styrene-b-ethylene oxide); Poly(lactide-b-ethylene oxide-b-lactide); Poly(lactone-b-ethylene oxide-b-lactone); a, w-Diacrylonyl Terminated poly(lactide-b-ethylene oxide-b-lactide); Poly(styrene-b-acrylic acid-b-styrene); Poly(styrene-b-butadiene (1,4addition) -b-styrene); Poly(styrene-b-butylene-b-styrene); Poly(styrene-b-n-butyl acrylate-b-styrene); Poly(styrene-b-t-butyl acrylate-b-styrene) [Polymer Source, Inc.]; Poly(styrene-b-ethyl acrylate-b-styrene); Poly(styrene-b-ethylene-b-styrene); Poly(styrene-b-isoprene-b-styrene); Poly(styrene-b-ethylene oxide-b-styrene); Poly(2-vinyl pyridine-b-t-butyl acrylate-b-2-vinyl pyridine); Poly(2-vinyl pyridine-b-butadiene(1,2addition)-b-2-vinyl pyridine); Poly(2-vinyl pyridine-b-styrene-b-2-vinyl pyridine); Poly(4-vinyl pyridine-b-t-butyl acrylate-b-4-vinyl pyridine); Poly(4-vinyl pyridine-b-methyl methacrylate-b-4-vinyl pyridine); Poly(4-vinyl pyridine-b-styrene-b-4-vinyl pyridine); Poly(butadiene-b-styrene-b-methyl methacrylate); Poly(styrene-b-acrylic acid-b-methyl methacrylate); Poly(styrene-b-butadiene-b-methyl methacrylate); Poly(styrene-b-butadiene-b-2-vinyl pyridine); Poly(styrene-b-butadiene-b-4-vinyl pyridine); Poly(styrene-b-t-butyl methacrylate-b-2-vinyl pyridine); Poly(styrene-b-t-butyl methacrylate-b-4-vinyl pyridine); Poly(styrene-b-isoprene-b-glycidyl methacrylate); Poly(styrene-b-a-methyl styrene-b-t-butyl acrylate); Poly(styrene-b-a-methyl styrene-b-methyl methacrylate); Poly(styrene-b-2-vinyl pyridine-b-ethylene oxide); Poly(styrene-b-2-vinyl pyridine-b-4-vinyl pyridine).

The above block copolymers can be used alone or in mixtures of two or more in the same or different classes. For example, in mixtures of two block copolymers measured in weight percent of the first polymer, such mixtures can comprise 10-15%, 15-20%, 20-25%, 25-30%, 30-35%, 35-40%, 40-45% or 45-50%. Where three polymers are used, the first can comprise 10-15%, 15-20%, 20-25%, 25-30%, 30-35%, 35-40%, 40-45% or 45-50% of the whole of the polymer components, and the second can 10-15%, 15-20%, 20-25%, 25-30%, 30-35%, 35-40%, 40-45% or 45-50% of the remainder.

Stated another way, the amount of each block copolymer in a mixture can vary considerably with the nature and number of the block copolymers used and the desired properties to be obtained. However, generally, each block copolymer of a mixture in accordance with the present invention will be present in an amount of at least about 10% based on weight of total polymers in the membrane or solution. These same general ranges would apply to membranes produced from one or more polymers, copolymers and/or mixtures with block copolymers. There may also be instances where a single polymer, copolymer or block copolymer may be “doped” with a small amount of a distinct polymer, copolymer or block copolymer, even as little as 1.0% by weight of the membrane to adjust the membrane's specific properties.

Embodiments of the invention include, without limitation, A-B, A-B-A or A-B-C block copolymers. The average molecular weight for triblock copolymers of A (or C) is, for example, 1,000 to 15,000 daltons, and the average molecular weight of B is 1,000 to 20,000 Daltons. More preferably, block A and/or C will have an average molecular weight of about 2,000-10,000 Daltons and block B will have an average molecular weight of about 2,000-10,000 Daltons.

If a diblock copolymer is used, the average molecular weight for A is between about 1,000 to 20,000 Daltons, more preferably, about 2,000-15,000 Daltons. The average molecular weight of B is between about 1,000 to 20,000 Daltons, more preferably about 2,000 to 15,000 Daltons.

Preferably, the block copolymer will have a hydrophobic/hydrophilic balance that is selected to (i) provide a solid at the anticipated operating and storage temperature and (ii) promote the formation of membrane-like structures rather than micelles. More preferably, the hydrophobic content (or block) exceeds the hydrophilic content (or block). Thus, at least one block of the diblock or triblock copolymers is preferably hydrophobic. While wettable membranes are possible, preferably the content of hydrophobic and hydrophilic synthetic polymeric materials will render the membrane sparingly wettable.

As described above, in one preferred embodiment of the present invention, there is provided a biocompatible membrane produced using a mixture of synthetic polymer materials. Such mixtures can be a mixture of two or more block copolymers that are identical but for the molecular weight of their respective blocks. For example, a biocompatible membrane can be produced using a mixture of two block copolymers, both of which are poly(2-methloxazoline)-polydimethylsiloxane-poly(2-methloxazoline), one of which having an average molecular weight of 2 kD-5 kD-2 kD and the other 3 kD-7 kD-3 kD and the ratio of the first block copolymer to the second is about 67% to 33% of the total synthetic polymer material used w/w. This shorthand reference means that the majority block copolymer's first block has a molecular weight of about 2 thousand Daltons, the second block has a molecular weight of 5 thousand Daltons and the third block has a molecular weight of 2 thousand Daltons. The minority block copolymer has blocks of about 3 thousand, 7 thousand and 3 thousand Daltons, respectively.

Furthermore, two or more entirely different block copolymers can be used and mixtures of different block copolymers and identical block copolymers that differ only in the size of their respective blocks are also contemplated. But mixtures are not limited to block copolymers.

Polymers and copolymers can be used, alone, in combination, and in combination with block copolymers in accordance with the present invention to produce biocompatible barriers and membranes having the properties described herein. Polymers and copolymers useful are preferably solid at room temperature (about 25° C.). They can be dissolved in solvents or solvent systems that can accommodate any other synthetic polymer material used, any additive used, and the polypeptide used. Polymers and copolymers useful in producing biocompatible membranes can include, without limitation polystyrenes, polyalkyl and polydialkyl siloxanes such as polydimethylsiloxane, polyacrylates such as polymethylmethacrylate, polyalkenes such as polybutadiene, polyalkylenes and polyalkylene glycols, sulfonated polystyrene, polydienes, polyoxiranes, poly(vinyl pyridines), polyolefins, polyolefin/alkylene vinyl alcohol copolymers, ethylene propylene copolymers, ethylene-butene-propylene copolymers, ethyl vinyl alcohol copolymers, perfluorinated sulfonic acids, vinyl halogen polymers and copolymers such as copolymers of vinyl chloride and acrylonitrile, methacrylic/ethylene copolymers and other soluble but generally hydrophobic polymers and copolymers all in a molecular weight of between about 5,000 and about 500,000. Particularly preferred polymers include: poly(n-butyl acrylate); poly(t-butyl acrylate); poly(ethyl acrylate); poly(2-ethyl hexyl acrylate); poly(hydroxy propyl acrylate); poly(methyl acrylate); poly(n-butyl methacrylate); poly(s-butyl methacrylate); poly(t-butyl methacrylate); poly(ethyl methacrylate) ; poly(glycidyl methacrylate); poly(2-hydroxypropyl methacrylate); poly(methyl methacrylate) poly(n-nonyl methacrylate); poly(octadecyl methacrylate); polybutadiene (1,4-addition); polybutadiene (1,2-addition); polyisoprene (1,4-addition); polyisoprene (1,2-addition and 1,4 addition); polyethylene; poly(dimethyl siloxane); poly(ethyl methyl siloxane); poly(phenyl methyl siloxane); polypropylene; poly(propylene oxide); poly(4-acetoxy styrene); poly(4-bromo styrene); poly(4-t-butyl styrene); poly(4-chloro styrene); poly(4-hydroxyl styrene); poly(a-methyl styrene); poly(4-methyl styrene); poly(4-methoxy styrene); polystyrene; Isotactic polystyrene; syndiotactic polystyrene; poly(2-vinyl pyridine); poly(4-vinyl pyridine); poly(2,6-dimethyl-p-phenylene oxide); poly(3-(hexafluoro-2-hydroxypropyl)-styrene); polyisobutylene; poly(9-vinyl anthracene); poly(4-vinyl benzoic acid); poly(4-vinyl benzoic acid sodium salt) ; poly(vinyl benzyl chloride); poly(3(4)-vinyl benzyl tetrahydrofurfuryl ether); poly(N-vinyl carbazole); poly(2-vinyl naphthalene) and poly(9-vinyl phenanthrene). Since polymers and copolymers are generally synthetic polymer materials, they may be used in the same amounts described previously for block copolymers and mixtures.

In a particularly preferred aspect of the present invention, the biocompatible membrane includes a synthetic polymer material, preferably at least one block copolymer (most preferably one that is, at least in part, amphiphilic) and a synthetic polymer material that can stabilize the biocompatible membrane. It has been discovered that certain polymers, most notably, hydrophilic polymers and copolymers capable of forming a plurality of hydrogen-bonds (“hydrogen bonding rich”) can stabilize the membrane. In the context of stabilizing polymers, the term “polymer” includes monomers, polymers and copolymers. “Hydrophilic” in this context means that the stabilizing polymer will dissolve or be solubilized in water or water miscible solvents. Without wishing to be bound to any particular theory of operation, it is believed that the use of such polymers can assist in functionally integrating polypeptides into the biocompatible membrane's structure. A stabilizing polymer imparts to a biocompatible membrane greater operating life and/or greater resistance to mechanical failure when compared to an identical biocompatible membrane produced without the stabilizing polymer when exposed to the same conditions. A stabilized biocompatible membrane wherein the synthetic polymer material includes a stabilizing polymer, used in an analyte sensor, for example, can have an increased operating life of at least about 10%, more preferably at least about 50%, most preferably at least about 100%.

Particularly preferred polymers capable of stabilizing the polypeptides in the biocompatible membranes of the present invention include: dextrans, polyalkylene glycols, polyalkylene oxides, polyacrylamides, and polyalkyleneamines. These stabilizing polymers (again including copolymers) have an average molecular weight which is generally lower than polymers and copolymers used as synthetic polymer materials. Their molecular weight generally ranges from about 1,000 Daltons to about 15,000 Daltons. Particularly preferred polymers capable of stabilizing biocompatible membranes include, without limitation, polyethylene glycol having an average molecular weight of between about 2,000 and about 10,000, polyethylene oxide having an average molecular weight of between about 2,000 and about 10,000, poly acrylamide having an average molecular weight of between about 5,000 and 15,000 Daltons. Other stabilizing polymers include: polypropylene, Poly(n-butyl acrylate); Poly(t-butyl acrylate); Poly(ethyl acrylate); Poly(2-ethyl hexyl acrylate); Poly(hydroxy propyl acrylate); Poly(methyl acrylate); Poly(n-butyl methacrylate); Poly(s-butyl methacrylate); Poly(t-butyl methacrylate); Poly(ethyl methacrylate) ; Poly(glycidyl methacrylate); Poly(2-hydroxypropyl methacrylate); Poly(methyl methacrylate); Poly(n-nonyl methacrylate); and Poly(octadecyl methacrylate).

The amount of stabilizing polymer(s) used in the biocompatible membranes is not critical so long as some measurable improvement in properties is realized and the functionality of the biocompatible membrane is not unduly hampered. Some trade-off of functionality and longevity is to be expected. However, generally, the amount of stabilizing polymer used, as a function of the total amount of synthetic polymer material found in the finished biocompatible membrane (by weight) is generally not more than one third, and typically 30% by weight or less. Preferably, the amount used is between 5 and about 30%, more preferably between about 5 and about 15% by weight of the synthetic polymer material in the finished membrane is used.

In addition to one or more polymers, copolymers and/or block copolymers, and/or stabilizing polymers, the synthetic polymer material of the invention can include at least one additive. Certain additives are used with polypeptides because the polypeptides prefer or require the presence of certain additives, such as lipids. Additives can include crosslinking agents and lipids, fatty acids, sterols and other natural biological membrane components and their synthetic analogs. These are generally added to the synthetic polymer material when in solution. These additives, if present at all, generally would be found in an amount of between about 0.50% and about 30%, preferably between about 1.0% and about 15%, based on the weight of the synthetic polymer material.

Where the biocompatible membrane incorporates cross-linking moieties, procedures useful for cross-linking include chemical cross-linking with radical-forming or propagating agents and cross-linking via photochemical radical generation with or without further radical propagating agents. Parameters can be adjusted depending on such conditions as the membrane material, the size of biocompatible membrane segments, the structure of the support, and the like. Care should be taken to minimize the damage to the polypeptide. One particularly useful method involves using peroxide at a neutral pH, followed by acidification.

Proton-Tunneling Membranes

One aspect of the present invention includes an analyte sensor having a barrier separating two compartments, the barrier being relatively proton impermeable, yet capable of participating in the transport of protons across the barrier. Without being bound by a particular theory of operation, it is believed that such transport is the result of proton-tunneling.

Yet another embodiment of the present invention is an analyte sensor containing a barrier with a PTM that is associated with at least one polypeptide capable of participating in the transport of protons from a first side to a second side of the membrane, which can include the formation of molecular structures that facilitate such transport. Preferably, the polypeptides are associated with the PTM such that they can participate in transporting protons across the PTM. In one embodiment, such polypeptides are provided as part of a separate biocompatible membrane, usually bonded to or coterminous with a PTM. In another embodiment, such polypeptides actually are part of a PTM. Such membranes are described in detail in the copending provisional application No. 60/415,686, entitled “Proton-Tunneling Membranes and Fuel Cells”, incorporated herein by reference to the extent permitted.

“Proton-tunneling” is a phrase that is used herein to describe an observed phenomena. Proton-tunneling membranes or barriers include one or more layers of synthetic polymer material (polymers, copolymers, block copolymers and mixtures of same), which are preferably impermeable to the flow of solids, liquids, gases, ions and, in particular, protons. At the very least, the majority of the flow of protons, as measured by the flow of current, is believed to be the result of proton-tunneling across these membranes and not mere proton permeability. The term “relatively proton impermeable,” as used herein, means that while a current or flow of protons occurs across the layer or membrane (under certain conditions), the minority of such current, if indeed any, is attributable to proton permeability. Proton-tunneling is believed to be the reason for transport of the remaining current, particularly in layers or membranes that do not include a polypeptide. However, even if proton-tunneling as an explanation is incomplete or inaccurate, the current that cannot be attributed to proton permeability is a measurable fact. It is this flow of protons to which the invention is directed and the phrase “relatively proton impermeable” is meant to reflect the fact that the synthetic polymer materials, when properly selected and formed into layers or membranes, will permit the flow of protons, the majority of which through a mechanism other than permeability. Yet these proton-tunneling membranes or barriers, even when completely proton impermeable as is the case in the most preferred embodiments of the present invention, are not dielectric; meaning that they will permit flow of protons when the proper materials are used and those materials are properly prepared as described herein. It has been discovered that when properly produced and used, layers of synthetic polymer materials capable of proton-tunneling, which would have been expected to be completely dielectric, in fact efficiently allow for the transporting of protons from one side of the layer to another. Membranes that meet the criteria of the present invention and exhibit such behavior are referred to herein as exhibiting proton-tunneling behavior and are proton-tunneling membranes or “PTMs.”

It is thought, for the reasons discussed herein, that proton-tunneling occurs because of cationic interaction between the protons and pi bonds of certain species within the polymer material of PTMs. This interaction is thought to be driven by excess positive charge. By modifying the amount of kinetic energy of the protons and/or by altering the nature of the proton affinity of the polymer materials used within the layer, one can adjust, and most preferably increase the rate and degree of proton transfer across the layer.

This phenomenon has been observed by the inventors to occur in certain polymeric material such as polystyrene based polymers, where the delocalized electron clouds within the benzene ring serves as a quantum well (or trap) for protons. Cationic binding with aromatic structures, as a phenomenon, has been noted in other contexts, such as with. cations and organic solvents, or amino acids. (See Dougherty, D. A., “Cation-Pi Interactions in Chemistry and Biology: A New View of Benzene, Phe, Tyr, and Trp.” Science 271: 163-168 (1996); Scrutton, N. S., and Raine, A. R. C., “Cation-Pi bonding and amino-aromatic interactions in the biomolecular recognition of substituted ammonium ligands” Biochem. J. 319: 1-8 (1996); Cubero, E., et al., “Is polarization important in cation-Pi interactions?” Proc. Natl. Acad. Sci. USA 95: 5976-80 (1998); Gallivan, J. P., and Dougherty, D. A., “Cation-Pi interactions in structural biology” Proc. Natl. Acad. Sci. USA 96:9459-64 (1999); Hunter, C. A., et al., “Substituent effects on cation-Pi interactions: A quantitative study” Proc. Natl. Acad. Sci. USA 99: 4873-76 (2002)).

It has now been discovered that in polymer layers in accordance with the present inventions, quantum wells, or continuous pathways between such wells, can be created by the presence of aromatic rings. It has been observed that when these rings bear substitute groups that strengthen the electron cloud, such as methyl groups, or in the case of non-conjugated rings or di- or polyaromatics, proton-tunneling activity is lowered. If, alternatively, the substitute groups weaken the electron cloud, as with halide-substituted styrenes, proton-tunneling activity is enhanced. These results are consistent with current research.

Without being limited by any particular theory of operation, it is believed that protons inside polymer membranes of the present invention can be trapped by quantum wells. The membranes themselves remain generally impermeable to protons unless there is an energetic proton available from one side of the membrane which enters the membrane through a tunneling effect. When one proton enters the membrane through tunneling, it is believed that another proton will exit from the other side of the membrane as the energy or protons are transferred. This may explain why the proton transport appears to be driven by excess charge, rather than by a concentration gradient.

A “quantum well” in accordance with the present invention is also a term used to explain a particular observation. It is not meant to be limiting, however. Quantum wells are a conceptualization of the energy barrier. The deeper the well, the more energy needed for tunneling. Certain polymer materials that are not useful in accordance with the present invention will not allow the transmission of protons from one side of a layer to another. Using a quantum well as an explanation, these polymer materials present a very deep well that can trap protons and quench proton-tunneling. Conceptually, no matter how much kinetic energy, within reason, a proton would have, it would be insufficient to tunnel through or out of such wells. Such materials are completely dielectric in accordance with the present invention. The shallower the well, the greater the probability that a proton can escape or tunnel through the layer. By adjusting the amount of kinetic energy of the protons and/or by adjusting the depth of the well (the affinity of the electron cloud of certain aromatic or resonance species for protons), one can increase or decrease the probability of proton-tunneling and increase the rate and density of proton flow.

To detect the strong binding of a protonic species, such as deuterium, to wells that are ‘too deep,’ i.e., soaking such materials in a deuterium-containing liquid, then exchanging the liquid and looking at the rate of deuterium loss from the material. Whereas those materials which characteristically contain wells that are ‘too shallow’ would neither allow the transfer of protons, nor measurably bind deuterium at a level above a control.

The PTM may be a single layer or multiple layers (including layers in intimate contact, or coterminous with each other), may be bound to a solid support (barrier) or stretched across or disposed within the apertures or pores of an otherwise dielectric material. Or a proton-tunneling barrier may be employed which is free standing.

In one embodiment, the invention includes a membrane having at least one layer of a synthetic polymer material separating a first compartment from a second compartment. The layer is relatively proton impermeable and not dielectric and is capable of participating in the transport of protons from the first side to the second side thereof. The synthetic polymer material used preferably includes at least one species that exhibits resonance and more particularly, at least one aromatic group. The synthetic polymer material preferably has a relatively low electrostatic binding energy and/or a relatively low amount of polarization energy.

In general terms, a PTM is made by selecting a synthetic polymer material that exhibits proton-tunneling and forming a layer from same having a first side and a second side. The layer must be capable of transporting protons from the first side of the layer to the second side of the layer.

In selecting synthetic polymer material a number of factors may be considered, including, without limitation, the depth of the quantum wells of the material under consideration, its propensity for proton-tunneling, whether or not a generally thin yet impermeable layer can be produced from that material, whether the material can produce a relatively thin layer that is at least relatively proton impermeable, the relative electrostatic binding energy and/or the relative polarization energy.

There are two basic criteria for PTMs in accordance with the present invention. First, they must be made out of a material that permits, facilitates or encourages the flow of protons, preferably even relatively low kinetic energy protons, through a mechanism other than permeability, and preferably through proton-tunneling. These materials should have relatively shallow quantum wells. While polymer layers that are relatively proton impermeable may be used, it is preferred that the polymer layer forming the membrane be as proton impermeable as possible. The less permeable the better. Therefore, it is preferred that one use a material that is “substantially totally proton impermeable,” which excludes all but incidental proton leakage or leaching from the layer. Second, the PTM must be produced in a way that permits proton-tunneling to occur.

As to the first criteria, not all materials allow for or facilitate proton-tunneling. Proton-tunneling activity, for example, is apparently blocked when the membrane includes nonconjugated unsaturated polymer components, such as butylenes. A copolymer of 1-2 butadiene and polystyrene (P2867 Polymer Source, Inc.) exhibits a drastic reduction of proton-tunneling activity at as low as 20% w/w in a membrane produced with P127 (Polymer Source, Inc), or 3G55 (BASF, Ludwigshafen, Germany) both of which are polystyrene-poly 1-4 butadiene-polystyrene triblock copolymers. A complete quenching of proton-tunneling activity occurs by 50% w/w. On the other hand, an otherwise identical membrane made completely of P127 or 3G55 exhibits significant proton-tunneling. Alternative ring structures, such as the nitrobenzene ring of polyvinylpyridine, also demonstrate proton-tunneling activity, but only when protonation sufficiently decreases the strength of the electron cloud. Thus an important aspect of the present invention is the selection of a polymeric material that permits proton transfer via proton-tunneling. The selected material should also be capable of being formed into layers/membranes of other solid/semisolid three dimensional structures which are proton impermeable.

Any synthetic polymer material containing an aromatic group, such as a benzene ring, may potentially be useful in accordance with the present invention. Other chemical species that do not, strictly speaking, possess an aromatic ring, but may share a resonance electron, may also be useful. Any synthetic polymeric material including a species that exhibits resonance is a possible candidate. This includes resonance hybrids. Aromatic compounds and compounds exhibiting resonance in this regard can include heterocyclic. rings, which exhibit resonance. Synthetic polymer materials may be homogenous, may be a copolymer or a block copolymer or a mixture. Not all of the synthetic polymer materials used, in a layer or membrane, be they monomers or blocks, need possess the capability of facilitating proton-tunneling.

As previously mentioned, compounds exhibiting resonance structure substituted with groups that strengthen the electron cloud such as methyl groups or generally electron positive groups can lower proton-tunneling activity. This can be thought of as digging a deeper well and thus making it harder for tunneling to occur. Alternatively, the use of substituents that weaken the resonance electron cloud such as electronegative groups like halides and electron withdrawing species such as hydroxy groups can enhance proton-tunneling or, to complete the analogy, provide a shallower well. Other compounds exhibiting an aromatic character but not generally conducive to proton-tunneling may be rendered useful in accordance with the present invention by increasing substitution with electronegative species or with sufficient protonation to decrease the strength of the electron cloud.

For example, membranes formed from a pure polystyrene-based homopolymer (M.W. 250,000 -Acros Organics, Lot No. A014302901, Geel, Belgium) produced by a solvent evaporation methodology as discussed herein, demonstrated proton-tunneling activity. A particularly useful polystyrene-based membrane can also be produced from a polystyrene-block copolymer such as 3G55, where polystyrene is formed with a flexible block, allowing a strong yet deformable membrane to be formed.

Testing the degree of proton-tunneling and indeed the degree of proton permeability, if any, can be done in a conventional manner. One preferred way is to produce a test cell having preferably a Zn or Al metal anode and a PTM. The test cell is operated for a predetermined period of time, generally about an hour or more, under a predetermined load, such as from 2-20 ohms, after first having measured the pH of the cathode compartment (measuring the pH of the electrolyte in the cathode compartment). Current and voltage are measured over that time and pH of the cathode compartment is measured at the end, once the load has been removed. From any measurable change in pH and the volume of the catholyte (electrolyte in the cathode compartment), one can calculate the number of protons crossing the membrane from anode to cathode via permeability. The total number of protons, the area of membrane, and the run time of the test, can be used to calculate the permeability to protons. The net number of protons crossing from anode to cathode via the membrane during the test is analogous to a current flow. The current due to proton permeability can be subtracted from the measured current output of the test cell and the difference is the current that can be attributed to proton-tunneling. As is generally true throughout, “current”, the flow of electrons over time, can be used to measure the ion flow (including protons)in a test cell (and across membranes)and therefore “current” is often used herein to describe the flow of protons as well. Thus, a ;material which will not permit the flow of protons is, for the purposes of this application, dielectric.

In general, the layers or membranes in accordance with the present invention are relatively proton impermeable and thus will have the majority of any current flowing across them attributed to something other than proton permeability. Preferably proton-tunneling will participate in the majority, and even more preferably, the overwhelming majority of the flow of protons. However, preferably, the relative proportion of current from other than permeability, such as proton-tunneling as compared to proton permeability will be much greater than 1:1. More preferably, a current ratio of at least 10:1 is observed (proton-tunneling:proton permeability). More preferably, at least 100:1 and even more preferably at least 1000:1. Most preferably, all of the current is attributable to something other than permeability and preferably to proton-tunneling. Of course, this would mean that the PTM is substantially totally proton impermeable. Generally polymers useful in accordance with the present invention will have either a relatively low electrostatic binding energy or a relatively low amount of polarization energy. Preferably, both are low. Indeed, a material that has both a relatively high electrostatic binding energy and a relatively high amount of polarization energy will usually not be useful to form a PTM for use in a PTM based analyte sensor. Conversely, if both the electrostatic binding energy and the amount of polarization energy are relatively low, then the material under consideration is a good candidate for use in a PTM. Preferably, for a PTM that will be used in an analyte sensor of the present invention, the electrostatic binding energy is less than about 19.3 Kcal/mole and more preferably, 15.0 Kcal/mole or less. The amount of polarization energy is preferably less than about 16.2 Kcal/mole and more preferably; 10.0 Kcal/mole or less. Both electrostatic binding energy and polarization energy figures are measured and calculated using the techniques and computations described in Cubero et al., “Is polarization important in cation-Pi interactions?” 95 Proc. Natl. Acad. Sci. USA (1998) 5976-80, the text of which is hereby incorporated by reference for purposes of describing methodologies for measuring and calculating both electrostatic binding energy and polarization energy.

Examples of synthetic polymer materials exhibiting these properties include: Triblock copolyampholytes from 5-(N,N-dimethylamino) styrene [Bieringer et al., Eur. Phys. J.E. 5:5-12, 2001. Among such polymers are Ai₁₄S₆₃A₂₃, Ai₃₁S₂₃A₄₆, Ai₄₂S₂₃A₃₅, Ai₅₆S₂₃A₂₁, Ai₅₇S₁₁A₃₂]; styrene-ethylene/butylene-styrene triblock copolymer [(KRATON) G 1650, a 29% styrene, 8000 solution viscosity (25 wt-% polymer), 100% triblock styrene-ethylene/butylene-styrene (S-EB-S) block copolymer; (KRATON) G 1652, a 29% styrene, 1350 solution viscosity (25 wt-% polymer), 100% triblock S-EB-S block copolymer; (KRATON) G 1657, a 4200 solution viscosity (25 wt-% polymer), 35% diblock S-EB block copolymer. The styrene-ethylene/propylene (S-EP) types and are commercially available under the tradenames (KRATON) G 1726, a 28% styrene, 200 solution viscosity (25 wt-% polymer), 70% diblock S-EP block copolymer; (KRATON) G-1701X a 37% styrene,>50,000 solution viscosity, 100% diblock S-EP block copolymer; and (KRATON) G-1702X, a 28% styrene,>50,000 solution viscosity, 100% diblock S-EP block copolmyer all available from the Shell Chemical Company, Houston, Tex., USA]; poly(styrene-b-butadiene-b-styrene) triblock copolymer [Commonly used thermoplastic elastomers, includes Styrolux from BASF, Ludwigshafen, Germany]; azo-functional styrene-butadiene-HEMA triblock copolymer, amphiphilic triblock copolymer carrying polymerizable end groups; poly(isoprene-block-styrene-block-dimethylsiloxane) triblock copolymer; poly(ethylene oxide)-block-polystyrene-block-poly(ethylene oxide) triblock copolymer; styrene-isoprene-styrene triblock copolymer [Japan Synthetic Rubber Co., MW=140 kg/mol, Block ratio of PS/PI=15/85]; [Ionomers of poly(styrene-co-4-styrene sulfonic acid or its salt); ionomers of Poly(styrene-co-N-methyl 2-vinyl pyridinium iodide); ionomers of poly(styrene-co-N-methyl 4-vinyl pyridinium iodide); ionomer of Poly(styrene-co-metal acrylate); ionomer of poly(styrene-co-metal methacrylate); ionomer of poly(styrene-co-metal 4-vinyl benzoate); bithiophene Labeled polystyrene; bromo-bithiophene labeled polystyrene; three-arm polystyrene; four-arm polystyrene eight-arm polystyrene; amino terminated poly(styrene-b-isoprene); amino terminated polystyrene; carboxy terminated polystyrene; carboxyl chloride terminated polystyrene; chloro terminated polystyrene; dimethyl chlorosilane terminated polystyrene; dimethyl silane terminated polystyrene; hydroxy terminated polystyrene; sulfonic acid sodium salt terminated polystyrene; sulfonic acid terminated polystyrene; thiol terminated polystyrene; vinyl terminated polystyrene; α, ω-dicarboxy terminated polystyrene; α, ω-dihydroxy terminated polystyrene; α-hydroxyl-ω-styrene terminated polystyrene; α-hydroxyl-ω-amino terminated polystyrene; α-hydroxyl-ω-carboxyl terminated polystyrene; α, ω-disulfonic acid terminated polystyrene; amino terminated poly(2-vinyl pyridine); carboxy terminated poly(2-vinyl pyridine); chloro terminated poly(2-vinyl pyridine); dimethyl chlorosilane terminated poly(2-vinyl pyridine); hydroxy terminated poly(2-vinyl pyridine); thiol terminated poly(2-vinyl pyridine); vinyl terminated poly(2-vinyl pyridine); α, ω-dihydroxy terminated poly(2-vinyl pyridine); α, ω-dicarboxy terminated poly(2-vinyl pyridine); carboxy terminated poly(4-vinyl pyridine); hydroxy terminated poly(4-vinyl pyridine); vinyl terminated poly(4-vinyl pyridine); random copolymer poly(styrene-co-4-bromostyrene); random copolymer poly(styrene-co-t-butyl-4-vinyl benzoate); random copolymer poly(styrene-co-t-butyl methacrylate); random copolymer poly(styrene-co-butadiene); random copolymer poly(styrene-co-p-carboxyl chloro styrene); random copolymer poly(styrene-co-p-chloro methyl styrene); random copolymer poly(styrene-co-methyl methacrylate); random copolymer poly(styrene-co-4-OH styrene); random copolymer poly(styrene-co-4-vinyl benzoic acid); alternating copolymer poly(carbo tert.butoxy α-methyl styrene-alt-maleic anhydride); alternating copolymer poly(a-methyl styrene-alt-methyl methacrylate); alternating copolymer poly(styrene-alt-methyl methacrylate); poly(butadiene-b-styrene-b-methyl methacrylate); poly(styrene-b-acrylic acid-b-methyl methacrylate); poly(styrene-b-butadiene-b-methyl methacrylate); poly(styrene-b-butadiene-b-2-vinyl pyridine); poly(styrene-b-butadiene-b-4-vinyl pyridine); poly(styrene-b-t-butyl acrylate-b-methyl methacrylate); poly(styrene-b-t-butyl methacrylate-b-2-vinyl pyridine); poly(styrene-b-t-butyl methacrylate-b-4-vinyl pyridine); poly(styrene-b-isoprene-b-glycidyl methacrylate); poly(styrene-b-a-methyl styrene-b-t-butyl acrylate); poly(styrene-b-a-methyl styrene-b-methyl methacrylate); poly(styrene-b-2-vinyl pyridine-b-ethylene oxide); poly(styrene-b-2-vinyl pyridine-b -4-vinyl pyridine; poly(t-butyl acrylate-b-styrene-b-t-butyl acrylate); poly(t-butyl methacrylate-b-styrene-b-t-butyl methacrylate); poly(methyl methacrylate-b-styrene-b-methyl methacrylate); poly(methyl methacrylate-b-2-vinyl pyridine-b-methyl methacrylate); poly(butadiene(1,4 addition)-b-styrene-b-butadiene(1,4 addition)); poly(ethylene oxide-b-styrene-b-ethylene oxide); poly(styrene-b-acrylic acid-b-styrene); poly(styrene-b-butadiene (1,4 addition)-b-styrene); poly(styrene-b-butylene-b-styrene); poly(styrene-b-n-butyl acrylate-b-styrene); poly(styrene-b-t-butyl acrylate-b-styrene); poly(styrene-b-ethyl acrylate-b-styrene); poly(styrene-b-ethylene-b-styrene); poly(styrene-b-isoprene-b-styrene); poly(styrene-b-ethylene oxide-b-styrene); poly(2-vinyl pyridine-b-t-butyl acrylate-b-2-vinyl pyridine); Poly(2-vinyl pyridine-b-butadiene(1,2 addition) -b-2-vinyl pyridine); Poly(2-vinyl pyridine-b-styrene-b-2-vinyl pyridine); poly(4-vinyl pyridine-b-t-butyl acrylate-b-4-vinyl pyridine); poly(4-vinyl pyridine-b-methyl methacrylate-b-4-vinyl pyridine); poly(4-vinyl pyridine-b-styrene-b-4-vinyl pyridine); poly(isoprene-b-N-methyl 2-vinyl pyridinium iodide); poly(butadiene-b-N-methyl 4-vinyl pyridinium iodide); poly(styrene-b-acrylic acid); poly(styrene-b-acrylamide); poly(styrene-b-cesium acrylate); poly(styrene-b-sodium acrylate); poly(styrene-b-methacrylic acid); poly(styrene-b-sodium methacrylate); poly(styrene-b-N-methyl 2-vinyl pyridinium iodide); poly(styrene-b-N-methyl-4-vinyl pyridinium iodide); poly(2-vinyl pyridine-b-ethylene oxide); poly(N-methyl 2-vinyl pyridinium iodide-b-ethylene oxide); poly(N-methyl 4-vinyl pyridinium iodide-b-methyl methacrylate); poly(t-butyl acrylate-b-2-vinyl pyridine); poly(t-butyl acrylate-b-4-vinylpyridine); poly(2-ethyl hexyl acrylate-b-4-vinyl pyridine); poly(t-butyl methacrylate-b-2-vinyl pyridine); poly(t-butyl methacrylate-b-4-vinyl pyridine); poly(butadiene-b-4-vinyl pyridine); poly(isoprene(1,4 addition)-b-2-vinyl pyridine); poly(isoprene(1,2 addition)-b-4-vinyl pyridine); poly(isoprene(1,4 addition)-b-4-vinyl pyridine); poly(ethylene-b-2-vinyl pyridine); poly(ethylene-b-4-vinyl pyridine); poly(isobutylene-b-4-vinyl pyridine); poly(styrene-b-butadiene(1,4 addition)); poly(styrene-b-n-butyl acrylate); poly(styrene-b-t-butyl acrylate); Poly(styrene-b-t-butyl methacrylate); poly(styrene-b-n-butyl methacrylate); poly(styrene-b-t-butyl styrene); poly(styrene-b-ε-caprolactone); poly(styrene-b-cyclohexyl methacrylate); Poly(styrene-b-N,N-dimethyl acrylamide); poly(styrene-b-N,N-dimethyl amino ethyl methacrylate); poly(styrene-b-dimethylsiloxane); poly(styrene-b-glycidyl methacrylate); poly(styrene-b-2-hydroxyethyl methacrylate); poly(styrene-b-2-hydroxyethyl methacrylate with cholesteryl chloroformate); poly(styrene-b-N-isopropyl acrylamide); poly(styrene-b-isoprene(1,4 addition)); poly(styrene-b-L-lactide); poly(styrene-b-methyl acrylate); poly(styrene-b-methyl methacrylate); poly(styrene-b-n-propyl methacrylate); Poly(styrene-b-methyl methacrylate (isotactic)); Poly(styrene-b-2-vinyl pyridine); poly(styrene-b-4-vinyl pyridine); tapered block copolymer poly(styrene-b-butadiene); tapered block copolymer poly(styrene-b-ethylene); poly(2-vinyl naphthalene-b-methyl methacrylate); poly(2-vinyl pyridine-b-e-caprolactone); poly(2-vinyl pyridine-b-methyl methacrylate); Poly(2-vinyl pyridine-b-4-vinyl pyridine); poly(4-vinyl pyridine-b-methyl methacrylate); poly(2-vinyl N-methyl pyridinium iodide*); poly(4-vinyl-N-methylpyridinium iodide*); Poly(styrene sulfonic acid); poly(4-acetoxy styrene); poly(4-bromo styrene); poly(4-t-butyl styrene); poly(4-chloro styrene); poly(4-hydroxyl styrene); poly(a-methyl styrene); poly(4-methyl styrene); poly(4-methoxy styrene); polystyrene; polystyrene broad distribution; isotactic polystyrene; syndiotactic polystyrene; poly(2-vinyl pyridine); poly(4-vinyl pyridine); poly(3-(hexafluoro-2-hydroxypropyl)-styrene); poly(4-vinyl benzoic acid) ; poly(vinyl benzyl chloride); poly(3(4)-vinyl benzyl tetrahydrofurfuryl ether); poly(N-vinyl carbazole) ; all of the above available from Polymer Source, Inc., Dorval, Quebec].

More preferably, nonrandom copolymers are used. The use of particularly well ordered materials such as isotactic or syndiotactic polystyrene to form membranes in accordance with the present invention may provide an even higher density of proton-tunneling fluxes. See U.S. Pat. No. 4,980,101.

As with biocompatible membranes discussed previously, it may be desirable to use stabilizing polymers with PTMs of the present invention, including those produced from nonrandom block copolymers, and the same principles, methods, and stabilizers may generally be employed. In a particularly preferred aspect of the present invention, the PTM and/or any biocompatible membrane produced may include a synthetic polymer material, preferably at least one block copolymer (most preferably one that is, at least in part, amphiphilic) and a synthetic polymer material that can stabilize the membrane.

The amount of stabilizing polymer(s) used is not critical so long as some measurable improvement in properties is realized and the functionality is not unduly hampered. Some trade of functionality and longevity is to be expected. However, generally, the amount of stabilizing polymer used, as a function of the total amount of synthetic polymer material found in the finished membrane (by weight) is generally not more than one-third, and typically 30% by weight or less. Preferably, the amount used is between 5 and about 30%, more preferably between about 5 and about 15% by weight of the synthetic polymer material in the finished membrane is used.

Returning to the discussion of the polymeric materials used to produce PTMs, the above polymers, copolymers and block copolymers can be used alone or in mixtures of two or more in the same or different classes, similar to the production of biocompatible membranes.

For example, in mixtures of two block copolymers measured in weight percent of the first polymer, such mixtures can comprise 10-15%, 15-20%, 20-25%, 25-30%, 30-35%, 35-40%, 40-45% or 45-50%. Where three polymers are used, the first can comprise 10-15%, 15-20%, 20-25%, 25-30%, 30-35%, 35-40%, 40-45% or 45-50% of the whole of the polymer components, and the second can 10-15%, 15-20%, 20-25%, 25-30%, 30-35%, 35-40%, 40-45% or 45-50% of the remainder.

Stated another way, the amount of each polymer in a mixture can vary considerably with the nature and number of the polymers used and the desired properties to be obtained. However, generally, each polymer of a mixture in accordance with the present invention will be present in an amount of at least about 10% based on weight of total polymers in the membrane or solution. These same general ranges would apply to PTMs produced from one or more polymers, copolymers and/or mixtures with block copolymers. There may also be instances where a single polymer, copolymer or block copolymer may be “doped” with a small amount of a distinct polymer, copolymer or block copolymer, even as little as 1.0% by weight of the membrane to adjust the membrane's specific properties.

The PTMs may include, without limitation, A-B, A-B-A or A-B-C block copolymers. The average molecular weight for triblock copolymers of A (or C) is, for example, 1,000 to 15,000 daltons, and the average molecular weight of B is 1,000 to 20,000 daltons. More preferably, block A and/or C will have an average molecular weight of about 2,000-10,000 Daltons and block B will have an average molecular weight of about 2,000-10,000 daltons.

If a diblock copolymer is used, the average molecular weight for A is between about 1,000 to 20,000 Daltons, more preferably, about 2,000-15,000 Daltons. The average molecular weight of B is between about 1,000 to 20,000 Daltons, more preferably about 2,000 to 15,000 Daltons.

Preferably, the block copolymer will have a hydrophobic/hydrophilic balance that is selected to (i) provide a solid at the anticipated operating and storage temperature and (ii) promote the formation of membrane-like structures rather than micelles. More preferably, the hydrophobic content (or block) shall exceed the hydrophilic content (or block). Thus, at least one block of the diblock or triblock copolymers is preferably hydrophobic. While wettable membranes are possible, preferably the content of hydrophobic and hydrophilic synthetic polymeric materials will render the membrane sparingly wettable.

As described above, in one embodiment of the present invention, there is provided a PTM produced using a mixture of synthetic polymer materials. Such mixtures can be a mixture of two or more block copolymers that are identical but for the molecular weight of their respective blocks. For example, a biocompatible membrane can be produced using a mixture of two block copolymers, 3G55 and P127 (from Polymer Source) and the ratio of the first block copolymer to the second is about 67% to 33% of the total synthetic polymer material used w/w.

Of course, two or more entirely different block copolymers can be used and mixtures of different block copolymers and identical block copolymers that differ only in the size of their respective blocks are also contemplated. But mixtures are not limited to block copolymers.

Polymers and copolymers can be used, alone, in combination, and in combination with block copolymers in accordance with the present invention to produce PTMs having the properties described herein. Polymers and copolymers useful are preferably solid at room temperature (about 25° C.) They can be dissolved in solvents or solvent systems that can accommodate any other synthetic polymer material used, any additive used, and a polypeptide, if used.

In addition to one or more polymers, copolymers and/or block copolymers, and/or stabilized polymers, the synthetic polymer material of the invention can include at least one additive. Additives can include crosslinking agents and lipids, fatty acids, sterols and other natural biological membrane components and their synthetic analogs. These are generally added to the synthetic polymer material when in solution. These additives, if present at all, generally would be found in an amount of between about 0.50% and about 30%, preferably between about 1.0% and about 15%, based on the weight of the synthetic polymer material.

Where the barrier, a PTM or biocompatible membrane, incorporates cross-linking moieties, procedures useful for cross-linking include chemical cross-linking with radical-forming or propagating agents and cross-linking via photochemical radical generation with or without further radical propagating agents. Parameters can be adjusted depending on such conditions as the membrane material, the size of biocompatible membrane segments, the structure of the support, and the like. Care should be taken to minimize the damage to the polypeptide used, if any. One particularly useful method involves using peroxide at a neutral pH, followed by acidification.

Polystyrene-based block copolymers with a flexible block allow strong, yet deformable membranes to be formed which demonstrate proton-tunneling activity. It is possible to increase the level of proton-tunneling activity in some block-copolymer based membranes via doping the preparation with homopolymers that exhibit proton-tunneling activity, including polystyrene, polyfluorostyrene, polychlorostyrene, or polybromostyrene.

It is anticipated that other aromatic polymer sidechains, as well as alternate substituents of styrenic polymers will demonstrate such proton-tunneling activity, including but not limited to: polyfluorostyrene, poly(difluoro)styrene, poly(trifluoro)styrene, poly(dibromoethyl)styrene, polyaminostyrene, polyphenol, pyridine ring substituents similar to those found to be active with styrene, including chloro and fluoro, etc., cyclopentene or cyclopentadienes and their derivatives, pyrrolidine and its derivatives, or any conjugated ring system that exhibits affinity for or the capacity to reversibly bond with protons.

Merely selecting the proper polymer material, however, is generally insufficient. It has been found that the way in which the layers or membranes are produced plays a significant role in whether or not a PTM can be created. That is to say that even the use of materials that have been found to be highly conducive to proton-tunneling may not be sufficient to produce PTMs in accordance with the present invention.

Generally, a PTM in accordance with the present invention should be as thin as possible while still maintaining structural integrity and proton impermeability, preferably substantially total proton impermeability. One method of accomplishing this goal is by casting very thin films onto other surfaces that will act as supporting structures. For example, a membrane or layer can be cast onto the surface of a porous material or a material into which apertures or holes have been formed, such as by drilling. The pores or apertures allow access to a portion of one side of the layer, while the other side may be completely exposed. Instead of casting a single layer across the entire surface, smaller membranes may be cast or placed adjacent each aperture or pore. In another alternative, a membrane or layer can be formed within an aperture or pore.

Alternatively, membranes can be cast on a solid surface and removed therefrom and placed onto or in some other form of solid support. Polymeric monolayers, or bilayers, are desirable for such applications.

Thicker layers can be produced as well. Generally, these layers can have a thickness of about 5 microns or more, preferably, however, they will be as thin as possible. The upper limit is unimportant as long as sufficient proton-tunneling activity is observed. These measurements are taken at the widest point of the membrane (the greatest distance between points on opposing surfaces). However, when membranes of this type are employed, it is preferred that they contain a structure such as that illustrated in FIG. 8. FIG. 8 illustrates in cross-section, an embodiment of a PTM, 100. The membrane contains a number of pores 104, which define relatively narrower portions of the membrane or interfaces 106. These interfacial areas 106 are significantly thinner than the surrounding portions 102 of membrane 100.

It is believed that these interfacial portions 106 are the site of proton-tunneling or, at least, a greater proportion of proton-tunneling. The relatively thin interfaces 106 created by pores 104 may create thousands relatively thin membrane barriers per square centimeter, while the relatively thicker portions 102 act as supporting superstructure to provide structural integrity. In this way, membrane 100 resembles a foam containing pores. “Pores” in this context does not equate to channels that run or traverse the thickness of membrane 100, as the membrane must be proton impermeable and preferably substantially totally proton impermeable. Such channels would increase permeability and are therefore generally undesirable. The width, depth and number of pores 104 need not be consistent or ordered. However, preferably, membrane 100 is formed so as to maximize the content of relatively thin portions 106.

Another methodology that can be employed in accordance with the present invention to create PTMs containing large relative proportion of interfacial areas 106 as illustrated in FIG. 8 is the use of microparticles 108. Microparticles can be made of anything including other polymeric materials, corn starch, polystyrene and isotactic polystyrene. Indeed, these microparticles can be made of materials that are themselves even more conducive to proton-tunneling than the membrane material itself. Preferred particles are produced using materials that contains a homocyclic, heterocyclic, polycyclic, aromatic or mixed carbon based ring structures. As shown in FIG. 8, the position occupied by various microparticles 108 may or may not have an influence on proton-tunneling. Microparticle 108a, for example, is completely encased within the polymer material of membrane 100. This positioning greatly reduces access by a proton to microparticle 108a. It is therefore less likely to provide benefit to proton-tunneling. Microparticle 108b, however, is disposed in one surface of membrane 100, which opposes a pore 104, but does not create a hole through membrane 100. This results in a relative narrowing of membrane 100 and is believed to create an interface region 106 as previously described. It is not clear whether proton-tunneling is facilitated by the actual particle or by small gaps or paths between the material of membrane 100 and the particle itself, which allow access to relatively thinner interfacial regions 106. It has been shown, however, that the use of such particulate matter can improve proton-tunneling.

Generally, these microparticles should have at least one dimension that is greater than the thickness of layer/membrane 100 to ensure that they are not totally encased as illustrated by particle 108a. Generally, the size of the particles can range from about 10-50 microns. The amount of particles is not important as long as the membrane remains viable and provides advantages in terms of proton-tunneling over membranes with lesser amounts of same. However, a weight of 3-4 times the weight of the other membrane materials is usually a practical limit.

Another technique used in creating PTMs in accordance with the present invention is surface wetting. Surface wetting may overcome charge effects at the surface, which interfere with the transfer of protons or may do nothing more than facilitate the reduction of surface tension within the pores thus permitting more complete wetting, and therefore a more complete interface for protons to relatively thinner interfacial regions of the membrane. In any event, however, it has been found that the use of surface wetting techniques greatly enhances the ability to transport protons across a PTM.

Such surface wetting may be accomplished through the formation of the membranes incorporating wetting agents, the modification of the surface of the membranes to establish wettability, or by other means. By way of example, even simple treatment of the surfaces with water-miscible organic solvents, such as methanol, ethanol, propanol, isopropanol, etc. is sufficient to allow proton-tunneling activity. It is preferable, however, that the surface of the polymer membrane be stable to the addition of the wetting agent. Other treatments, such as coating the surface with polyvinyl alcohol, or glycerol, or with enzymes which alter the surface properties, as in U.S. Pat. No. 6,436,696 (wherein treatment with lipase improves the surface wettability of polyesters) are also possible.

Bonding or grafting of hydrophilic or amphiphilic polymers to the surface, as in U.S. Pat. Nos. 6,433,243 or 6,440571, or U.S. patents applications Nos. 20020004140, 20020120333, 20020061406, or 20020017487, or such procedures using super-critical carbon dioxide, as in U.S. patent application No. 20020051845 may also be used, provided these processes do not occlude the structures necessary for protons to enter the membrane, although this is not intended to imply a particular form.

Prior to the use of a wetting agent, it may be desirable to treat the membrane or one layer thereof with an acid followed by neutralization. The type of acid and its concentration are not critical as long as an improvement is observed between a membrane prepared with the acid wash and one prepared identically without the acid wash. Acids can include, without limitation: organic acids and mineral acids such as hydrochloric acid, sulfuric acid, nitric acid, ascorbic acid, citric acid. Generally, the acid will have a concentration of at least about one normal. The length of time of acid treatment is also not critical as long as the objectives described above are met and the structural integrity of the membrane is not compromised. However, generally, the treatment will last from a few seconds to a few minutes. It has been observed that at least with some of the membranes in accordance with the present invention this additional treatment step can shrink the membrane minimally, and increase its modulus and stiffness. Generally thereafter, wetting is used as described above.

Another method of promoting proton-tunneling and producing effective PTMs is the use of certain polypeptides within or in association with a PTM. In this regard, two interesting phenomena have been observed. First, when a PTM is placed in intimate contact with a biocompatible membrane composed of, for example, a block copolymer and a polypeptide such as NADH dehydrogenase (“Complex I”), protons are effectively transported across both layers. Indeed, the efficiency of transport of the biocompatible membrane is not compromised. Second, a membrane created as a single layer PTM as described herein and also including Complex I, for example, is also useful for transmitting protons. In FIG. 8, polypeptides 112 are shown associated with PTM 100. These polypeptides may be disposed in a portion of, or a single surface of, membrane 100 (see 110a), disposed within a pore (see 110b) or may traverse the membrane (see 110c). What is particularly interesting about this second embodiment is that the amount of protons transported from one surface of the layer to the other can exceed that which would be expected from the creation of the membrane with the same amount of Complex I, which had no proton-tunneling capabilities and the creation of a membrane having proton-tunneling abilities but no Complex I. These sorts of synergies are not uncommon in accordance with the present invention. In essence, this embodiment creates a biocompatible membrane from proton-tunneling materials having sufficiently shallow quantum wells.

Polypeptides that can be used in accordance with the present invention-whether in association with a PTM, or in a combined membrane having a PTM layer coterminous with a biocompatible membrane layer associated with polypeptides-wherein the polypeptides are capable of participating in the transporting of protons from a first side to a second side of the membrane, including participating in the formation of molecular structures that facilitate such transport. Polypeptides which are also useful in accordance with the present invention include any that, when added or in other ways associated with the PTM, facilitate proton-tunneling. Preferably, the polypeptides are associated with the PTM such that they can participate in transporting protons across the PTM.

Electron Transfer Mediators

An “electron carrier” refers to a molecule used to donate electrons in an enzymatic reaction. Electron carriers include, without limitation, reduced nicotinamide adenine dinucleotide (denoted NADH; oxidized form denoted (NAD or NAD+), reduced nicotinamide adenine dinucleotide phosphate (denoted NADPH; oxidized form denoted NADP or NADP+), reduced nicotinamide mononucleotide (NMNH; oxidized form NMN), reduced flavin adenine dinucleotide (FADH2; oxidized form FAD), reduced flavin mononucleotide (FMNH2; oxidized form FMN), reduced coenzyme A, and the like. Electron carriers include proteins with incorporated electron-donating prosthetic groups, such as coenzyme A, protoporphyrin IX, vitamin B12, and the like. Further, electron carriers include gluconic acid (oxidized form: glucose), oxidized alcohols (e.g., ethylaldehyde), and the like.

An “electron transfer mediator” refers to a composition which facilitates transfer to an electrode of electrons released from an electron carrier.

Electron transfer mediators are known in other contexts in the art, as illustrated in: Wingard et al., Enzyme Microb. Technol. 4:137-142, 1982 (methyl viologen); Palmore et al., J. Electroanalytical Chem. 443: 155-161, Feb. 10, 1998 (1,1′-dibenzyl-4,4′-dipyridinium dichloride, benzyl viologen); Matsue et al., Biochem. Biophys. Acta, 1038: 29-38, 1990 (N,N,N′,N′-tetramethyiphenylenediamine, TMPD). Among further electron transfer mediators are methyl viologen, TMPD and phenazine methosulfate (PMS), which have been successfully used in fuel cell devices using complex I.

Electron transfer mediators in some embodiments are used to transfer electrons from an electrochemical reduction at the barrier B to a spatially separated electrode. In some embodiments, these can be incorporated at side S2.

Exemplary Polypeptides

In some aspects of the analyte sensors of the present invention, polypeptides are employed which are capable of participating in the transport of a species across a biocompatible membrane or PTM once a specific analyte has been introduced. In some embodiments, the polypeptide may have catalytic activity, which is generally the transport of a chemical or proton across a biocompatible membrane from side S1 to side S2, or extraction of electrons that can be ferried to an electrode, for instance using an electron transfer mediator.

It should be noted that any reference to a “polypeptide” herein may include a single polypeptide, but also includes reference to a complex of polypeptides that together provide a functional unit capable of participating in the transport upon which the analyte sensor is based.

Where appropriate, side S1 includes components, such as enzymes or reagents that transform the analyte to a form which the polypeptide PP can act upon. Such enzymes can be immobilized in the vicinity of the polypeptide, PP.

Examples of useful polypeptides that can be associated with a synthetic polymer material, so as to form a biocompatible membrane or PTM in accordance with the present invention, and that can participate in one or both of the oxidation/reduction and transmembrane transport functions (molecules, atoms, protons, electrons) include, without limitation, NADH dehydrogenase (“complex I”) (e.g., from E. coli. Tran et al., “Requirement for the proton pumping NADH dehydrogenase I of Escherichia coli in respiration of NADH to fumarate and its bioenergetic implications,” Eur. J. Biochem. 244: 155, 1997), NADPH transhydrogenase, proton ATPase, and cytochrome, oxidase and its various forms. Further polypeptides include: glucose oxidase (using NADH, available from several sources, including a number of types of this enzyme available from Sigma Chemical), glucose-6-phosphate dehydrogenase (NADPH, Boehringer Mannheim, Indianapolis, Ind.), 6-phosphogluconate dehydrogenase (NADPH, Boehringer Mannheim), malate dehydrogenase (NADH, Boehringer Mannheim), glyceraldehyde-3-phosphate dehydrogenase (NADH, Sigma, Boehringer Mannheim), isocitrate dehydrogenase (NADH, Boehringer Mannheim; NADPH, Sigma), a-ketoglutarate dehydrogenase complex (NADH, Sigma) and proton-translocating pyrophosphates. Also included are succinate:quinone oxidoreductase, also referred to as “Complex II,” “A structural model for the membrane-integral domain of succinate:quinone oxidoreductases” Hagerhall, C. and Hederstedt, L., FEBS Letters 389; 25-31 (1996) and “Purification, crystallisation and preliminary crystallographic studies of succinate:ubiquinone oxidoreductase from Escherichia coli.” Tornroth, S., et al., Biochim. Biophys. Acta 1553; 171-176 (2002), heterodisulfide reductases, F(420)H(2) dehydrogenase, (Baumer et al., “The F420H2 dehydrogenase from Methanosarcina mazei is a Redox-driven proton pump closely related to NADH dehydrogenases.” 275 J. Biol. Chem. 17968 (2000)) or a formate hydrogenlyase (Andrews, et al., A 12-cistron Escherichia coli operon (hyf) encoding a putative proton-translocating formate hydrogenlyase system.” 143 Microbiology 3633 (1997)), Nicotinamide nucleotide transhydrogenases: “Nicotinamide nucleotide transhydrogenase: a model for utilization of substrate binding energy for proton translocation.” Hatefi, Y. and Yamaguchi, M., Faseb J., 10; 444-452 (1996), Proline Dehydrogenase: “Proline Dehydrogenase from Escherichia coli K12. ” Graham, S., et al., J. Biol. Chem. 259; 2656-2661 (1984), and Cytochromes including, without limitation, cytochrome C oxidase (crystallized with either undecyl-b-D-maltoside or cyclohexyl-hexyl-b-D-maltoside), Cytochrome bc1: “Ubiquinone at Center N is responsible for triphasic reduction of cytochrome bc1 complex.” Snyder, C. H., and Trumpower, B. L., J. Biol. Chem. 274; 31209-16 (1999), Cytochrome bo3: “Oxygen reaction and proton uptake in helix VIII mutants of cytochrome bo3.” Svensson, M., et al., Biochemistry 34; 5252-58 (1995), “Thermodynamics of electron transfer in Escherichia coli cytochrome bo3.” Schultz, B. E., and Chan, S. I., Proc. Natl. Acad. Sci. USA 95; 11643-48 (1998), and Cytochrome d: “Reconstitution of the Membrane-bound, ubiquinone-dependent pyruvate oxidase respiratory chain of Escherichia coli with the cytochrome d terminal oxidase.” Koland, J. G., et al., Biochemistry 23; 445-453 (1984), Joost and Thorens, “The extended GLUT-family of sugar/polyol transport facilitators: nomenclature, sequence characteristics, and potential function of its novel members (review)” 18 Mol. Membr. Biol. 247-56 (2001), and selective channel proteins including those disclosed in Goldin, A. L., “Evolution of voltage-gated Na(+) channels.” J. Exp. Biol. 205; 575-84 (2002), Choe, S., “Potassium channel structures.” Nat,. Rev. Neurosci. 3;115-21 (2002), Dimroth, P., “Bacterial sodium ion-coupled energetics.” Antonie Van Leeuwenhoek 65; 381-95 (1994), and Park, J. H. and Saier, M. H. Jr., “Phylogenetic, structural and functional characteristics of the Na—K—Cl cotransporter family.” J. Membr. Biol. 149; 161-8 (1996). All of the foregoing are hereby incorporated by reference.

Additionally, it is contemplated that genetically modified polypeptides, such as modified enzymes, can be used. One commonly applied technique for genetically modifying an enzyme is to use recombinant tools (e.g., exonucleases) to delete N-terminal, C-terminal or internal sequence. These deletion products are created and tested systematically using ordinary experimentation. As is often the case, significant portions of the gene product can be found to have little effect on the commercial function of interest. More focused deletions and substitutions can increase stability, operating temperature, catalytic rate and/or solvent compatibility providing enzymes that can be used in the invention. Of course it is possible to use mixtures of various polypeptides described herein as may be desirable.

Cytochrome P450s (CYPs)

Cytochrome P450 “oxidoreductases” (“CYPs”) utilize oxygen and an electron carrier (generally NADPH or NADH) to add oxygen to a substrate in a hydroxylation, epoxidation or peroxygenation reaction. An important subset of CYPs frequently react with drugs to, primarily, render these xenobiotic chemicals more water soluble and hence facilitating their excretion. CYPs can also act to activate or inactivate such drugs, or in some instances to create toxic derivatives of such drugs. Drugs can also act as inhibitors of CYPs, creating a potential for adverse drug interactions wherein a CYP that acts to remove a drug, and hence to establish the anticipated pharmacokinetics for a drug, is inactivated. Such inhibition results in the drug being resident in the body longer than anticipated, leading to drug build up and too great a possibility of toxicity. CYP inhibition can also prevent activation of another drug, potentially causing harm by disabling an important pharmacological intervention.

In humans, from the family of more than twenty CYP enzymes, CYP1A2, CYP2C9, CYP2C19, CYP2D6, CYP2E1 and three closely related “CYP3A” enzymes (CYP3A4, CYP3A5 and CYP3A7) account for the metabolism of nearly all clinically useful medications. CYP3A4 is believed to be one of the more clinically significant of the CYPs and is present in the small intestine as well as the liver, such that it has an early opportunity to act on orally administered medicaments.

An analytical cell of the present invention can be used to measure whether a chemical is acted upon by a given CYP, or, if the chemical is introduced to the analytical cell with a chemical that is acted upon by the CYP, whether the first chemical is an inhibitor or activator of the CYP.

CYP activity is, for example, detected by electron leakage to electron transfer mediators.

Sequence information on CYP1A2 can be found under Swiss-Prot Accession P05177; on CYP2C9 under Accession P11712; on CYP2C19 under Accession P33261; on CYP2D6 under Accession P10635; on CYP2E1 under Accession P05181; on CYP3A4 under Accession P08684; on CYP3A5 under Accession P20815; on CYP3A7 under Accession P24462. Reconstitution of recombinants into a membrane environment CYPs is an ordinary component of CYP assays. See, e.g., Hanna et al. J. Biol. Chem. 276:39553-39561, 2001.

Sugar Transporters

In prior work, a glucose transporter has been used to transport glucose across a biological membrane to a thin layer of aqueous gel affixed to an electrode. The biological membrane was also bonded to the gel. The gel, either a crosslinked gel or an aromatic polyamine-polymer, was doped with avidin and deposited at the metal electrode by electropolymerization. This layer (less than 10 nm thick) served as a submembrane compartment. The facilitated glucose transporter (GLUT-I) purified from human erythrocytes was integrated into a lipid membrane containing artificial biotinylated lipids and reacted with the activated surface of the glucose sensitive electrode. Neumann-Spallart et al., Appl. Biochem. Biotechnol. 68(3):153-69, 1997.

In the present invention, a sugar transporter is incorporated into a membrane M1 of the barrier. B1, which membrane is not supported upon an electrode. The transporter can be any facilitative transporter of a C5 to C9 sugar, or a molecule related thereto by one to two substitutions of a hydroxide by an amine (which may be acetylated) or hydrogen, by further oxidation to form a carboxylic acid (or salt thereof), by methylation or ethylation of one or more hydroxyls, by formation of a phosphoester at one or more hydroxyls, or by two of the foregoing molecules being joined at their anomeric carbons by an ether bridge formed by a dehydration reaction (as a whole, a class of molecules also generally referred to as “sugars”). Preferably, the transporter transports glucose or fructose, most preferably glucose. The transporter is preferably a member of the GLUTx family that includes GLUT1 (from erythrocytes), GLUT2 (from liver), GLUT3 (from brain), GLUT4 (from muscle), GLUT5 (from small intestine) and GLUT7 (from microsomes). A large number of these transporters have been cloned, and information on them can be found in Swiss-Prot database.

The transporter provides a selective entryway into detection side S2. At side S2, ordinary detection means can be employed, including electrodes on which glucose oxidase has been linked or absorbed. Or, because of the selectivity provided by the barrier B1, a less selective detection method can be used, including measuring an increase or decrease in optical rotation at an appropriate wavelength as measured at side S2, reaction with diphenylhydrazine to form a colored product, and the like.

Amino Acid Transporters

A great number of amino acid transporters have been characterized, and can be used in an analytical cell. Such amino acid transporters include transporters for gamma amino butyric acid, taurine, and other amino acids utilized in plants or animals. Where the transporter co-transports other molecules, such as sodium and chloride in the case of the glycine transporter Glyt1, the co-transported moieties placed at side S1 or S2 as appropriate. Or, such a transporter can be used to detect the co-transported substance, and the amino acid provided as needed to support the transport thus dependent upon the co-transported substance. Also, the co-transported moiety can be preferentially placed so that it is detectably transported to the other side when the amino acid is transported. Accordingly, in some embodiments, transport to side Si can provide the indication, or a supplementary indication, of activity. (These considerations on co-transported substances apply to any co-transporting transporter, not just to such amino acid transporters.)

That these amino acid transporters can be reconstituted in lipid bilayer analogous systems has been demonstrated by, for example, the reconstitution of solubilized “System A” amino acid transporters into liposomes described by Fafournoux et al., J. Biol. Chem. 264:4805-4811, 1989.

Again, in addition to highly specialized detection systems, such as systems utilizing amino acid catabolizing enzymes affixed to an electrode, the selectivity provided by the barrier B1 allows the use of less selective detections. Such detections include colorimetric detection for amines (e.g., reaction with ninhydrin) and fluorometric detection for amines (e.g., reaction with o-phthalaldehyde), and the like.

Electron and/or Proton Transporting Proteins

The presence of an electron carrier (as the analyte or as a result of catabolism of the analyte) can be detected with redox enzymes that transport electrons from such electron carriers. Use of an appropriate electron transfer mediator carries such electrons to a separately located electrode. Or, where the transporter co-transports protons, a change in pH can be detected.

Examples of particularly preferred redox enzymes providing one or both of the oxidation/reduction and proton pumping functions include, for example, NADH dehydrogenase (“complex I”), NADPH transhydrogenase, proton ATPase, and cytochrome oxidase and its various forms, and the like. The complex I NADH dehydrogenase (or, NADH:ubiquinone oxidoreductase), which is expressed from an operon, can be overexpressed in E. coli by substituting a T7 promoter in the operon to provide quantities useful in the invention.

Complex I can be isolated from over-expressing E. coli by the method described by Spehr et al., noted above, using solubilization with dodecyl maltoside.

Further redox enzymes can be artificially associated with a biocompatible membrane or PTM. For example, amphipathic molecules having a protein reactive moiety tethered to the hydrophilic end can be used to associate a polypeptide directly or indirectly to the biocompatible membrane or PTM. Indirect associations include for example, coupling avidin to the amphiphile to strongly associate a biotinylated enzyme. For this use, and other redox enzyme uses, a lipid soluble electron transfer mediator, such as coenzyme Q, can facilitate electron movement to side S2. As can be seen, some of the redox enzymes react in a manner dependent upon the presence of prospective analytes (or derivatives), such that generation of the reduced electron carrier is not the signal triggering event. In these cases, the reduced electron carrier is provided at side S1 in amounts sufficient to support the signal triggering reaction.

It will be recognized that the source of any polypeptide used in the invention can be a thermophilic organism providing a more temperature stabile polypeptide. For example, complex I can be isolated from Aquifex aeolicus in a form that operates optimally at 90° C., as described in Scheide et al., FEBS Letters 512: 80-84, 2002 (describing a preliminary isolation using the type of detergent extraction used elsewhere for complex I).

In one embodiment, the invention provides a toxicity screen whereby bioactive agents or prospective bioactive agents (e.g., drugs) are tested against multiple transporters for neurotransmitters.

Pore-Forming Polypeptides

Grmacidin is a pore-forming membrane polypeptide that is stable in organic solvents and is therefore conducive to incorporation into a biocompatible membrane. Gramacidin functions in pairs or groups with species, e.g., ions, passing through the membrane only when two or more gramacidin molecules are associated to form a pore. The ability of the membrane to allow transport of a species is determined by the number of associated pairs or groups of gramacidin molecules. An analyte sensor of the present invention can be used to measure whether an analyte or derivative of an analyte acts upon the gramacidin, or interacts with the gramacidin to inhibit the pore-forming function of the gramacidin pairs. In this embodiment of the present invention, species known to be capable of transport through the gramacidin pore can be introduced into the first compartment of an analyte sensor and the presence of this species in the second compartment indicates that the gramicidin pore is functioning to allow species transport. The presence of an analyte or derivative of an analyte that interacts with the gramacidin to disrupt the pores and the transport of the detected species. Detection, for example, by reduction or elimination of the signal in the second compartment indicates the presence of the analyte. Gramacidin can be incorporated into the membrane at mass ratios of polypeptide to polymer in amounts as low as about 1:50,000; preferably, useful amounts are about 1:100 to about 1:50,000.

Formation of Barriers Including Biocompatible Membranes and PTMs

The amount of polypeptide used will vary with the type of polypeptide used, the nature and function of the PTM or biocompatible membrane, the environment in which it will be used, and the polymeric material used, etc. In general, however, as long as some polypeptide is present and functional, and as long as the amount of polypeptide used does not prevent membrane formation or render the membrane unstable, and, if used in a PTM, as long as the proton-tunneling is enhanced, then any amount of polypeptide is useful. Generally, the amount of polypeptide will be at least about 0.01%, more preferably at least about 5%, even more preferably at least about 10%, and still more preferably at least about 20% and most preferably 30% or more by weight based on the final weight of the biocompatible membrane or PTM. The maximum amount is similarly not limited, except by the ability to form a stable and functional membrane. The amount of polypeptide to solvent can be as low as 0.001% w/v and as high as 50.0% w/v. Preferably, the concentration is from about 0.5% to about 5.0% w/v. More preferably the concentration is from about 1.0% to about 3.0% w/v. These ranges apply both to PTMs and biocompatible membranes.

Suitable solubilizing and/or stabilizing agents such as cosolvents, detergents and the like may also be needed, particularly in connection with the polypeptide solution. Solubilizing detergents are useful typically at the 0.01% to 1.0% concentration level, and more preferably up to about 0.5% is contemplated. Such detergents include ionic detergents: Sodium dodecyl sulfate, Sodium N-dodecyl sarcosinate, N-dodecyl Beta-D-glucopyranoside, octyl-Beta-D-glucopyranoside, dodecyl-maltoside, decyl, undecyl, tetradecyl-maltoside (in general, an alkyl chain of about 8 carbons or more bonded to a sugar as a general form of an ionic detergent) octyl-beta-D-glucoside and polyoxyethylene (9) dodecyl-ether, C₁₂E₉, as well as non-ionic detergents, such as Triton X-100, or Nonidet P-40. Also useful are certain polymers, typically diblock copolymers which exhibit surfactant properties, such as BASF's Pluronic series, or Disperplast (BYK-Chemie).

The solvent used in producing the synthetic polymer material solution is preferably selected to be miscible with both the water used (the polypeptide solution often includes water) and at least one of the synthetic polymer materials (polymer, copolymer and/or block copolymer). However, as described above, it is possible to form membranes using solvents or mixtures which are not water miscible. Note that while the use of solvents to produce solutions is preferred, the term “solution” as used herein generally encompasses suspensions as well.

When a block copolymer is used, the solvent should solubilize these synthetic polymer materials. While the synthetic polymer material may be relatively sparingly soluble in the solvent (less than 5% w/v), it is preferably more soluble than 5% w/v and generally, solubility is at least 5 to 10% w/v, preferably greater than 10% w/v synthetic polymer material to solvent.

Appropriate solvents may include, without limitation, low molecular weight aliphatic alcohols and diols of between 1 and 12 carbons such as methanol, ethanol, 2-propanol, isopropanol, 1-propanol, aryl alcohols such as phenols, benzyl alcohols, low molecular weight aldehydes and ketones such as acetone, methyl ethyl ketone, cyclic compounds such as benzene, cyclohexane, toluene and tetrahydrofuran, halogenated solvents such as dichloromethane and chloroform, and common solvent materials such as 1,4-dioxane, normal alkanes (C₂-C₁₂) and water. Solvent mixtures are also possible as long as the mixture has the appropriate miscibility, rate of evaporation and the other criteria described for individual solvents. Solvent components that have any tendency to form protein-destructive contaminants such as peroxides can be used as long as they can be appropriately purified and handled. Solvent typically comprises 30% v/v or more of the polypeptide/synthetic polymer material solution, preferably 20% v/v or more, and usefully 10% v/v or more.

If the biocompatible membranes or PTMs are to include “other materials” such as detergents, lipids (e.g. cardiolipin), sterols (e.g. cholesterol) or buffers and/or salts, those too would be added prior to formation of the membrane and they would be present in an amount of between about 0.01 and about 30%, preferably between about 0.01 and about 15% based on the weight of the finished biocompatible membrane. Other materials, as opposed to additives, are most often mixed with the polypeptide solutions, not the synthetic polymer solutions.

One technique for improving proton-tunneling in a PTM is the use of doping. Dopants, as opposed to particles, are soluble along with the primary synthetic polymer in the solvent or solvent combination employed. This can be accomplished by including homopolymers that exhibit proton-tunneling activity including, for example, polystyrene, polyfluorostyrene, polychlorostyrene or polybromostyrene up to about 20% by weight.

PTM formation, in particular, can be accomplished in several ways. One technique involves first creating a solution of the polymer to be used in an appropriate solvent. Solvents generally useful for creation of PTMs have been discussed. However, for most styrene polymers, aliphatic or aromatic organic solvents are required. To dissolve pellets of 3G55, mixtures of 50% v/v acetone with an alkane solvent (either pentane, hexane, heptane or octane) can be useful, though dissolution of STYROLUX 3G55 (polystyrene-polybutadiene-polystyrene triblock polymer) pellets in tetrahydrofuran is preferable. The concentration of useful solutions varies from 1% to 50% w/v, more preferably the concentration is from 5% to 10 % w/v.

The inclusion of non-dissolvable microparticles in a PTM, such as those formed of polyethylene, isotactic polystyrene, or cornstarch, dopants, etc., if contemplated, should be added to the polymer solution at this point.

Also possible is the inclusion of surfactants, such as dodecyl maltoside, Triton X-100, Nonidet P-40, sodium dodecyl sulfate, or polymeric surfactants such as Pluronic L-101 (BASF) in the membrane-forming solution. Such inclusion provides a wettable surface which does not necessarily require further surface activation. If the PTM is to include a polypeptide, that too should be added at this time. Polypeptides may be added directly to the membrane forming solution or, as discussed herein, may be placed in a separate solution for introduction first.

In one embodiment, the polymer solution may be contacted to apertures in a thin-sheet support of materials such as polyimide (Kapton), thermoformed polystyrene sheet or polysulfone. For the latter two materials, the support is also soluble in the solvent (THF) and bonding is therefore more complete than in the former case, where delamination can occur if the mechanical properties of the membrane-forming polymer and the support do not match well, such as with homopolymeric polychlorostyrene.

Apertures in a support can be as small as 1 micron, or as large as several millimeters without need for a delaminatable backing, such as polytetrafluoroethylene (Teflon-Dupont). With such a backing, apertures of more than 1 cm in diameter can be used.

It is also possible to cast unsupported membranes with these polymeric materials. Membranes of regular or irregular shapes have been cast with areas greater than a square inch, and larger membranes are possible.

All PTMs formed as above are allowed to form via solvent evaporation in a fume hood until visibly dry prior to vacuum drying for at least an additional 15 minutes.

Following drying, the surface of the PTM can be treated to allow a greater degree of wetting (especially where surfactants were not used to form the polymeric sheet). Such treatment involves primarily immersion in a bath of a wetting solution, or minimally coating or spraying the surfaces of the membrane with a wetting solution. Wetting solutions include alcohols (methanol, ethanol, propanol, isopropanol) or alcohol-water mixtures; surfactant solutions (up to 1% w/v of compounds as described above) or can include other treatments that render the surface of the membrane more hydrophilic. When polypeptides are included in the PTM, it may not be necessary to add surfactants at all. This may be because many useful polypeptides are isolated and provided commercially in solutions that already contain surfactants. It is believed, however, that the presence of enzymes capable of proton transport may eliminate the need for surface wetting entirely. One should also be cautious when using additional surface wetting agents in PTMs incorporating polypeptides as surfactants can denature polypeptides in certain instances.

Biocompatible membranes, and PTMs which include polypeptides, in accordance with the present invention, can be produced using any one of a number of conventional techniques used in the production of membranes from synthetic polymer materials, as long as the resulting biocompatible membranes and PTMs are useful as described herein. One method of forming biocompatible membranes, which is also useful in the formation of PTMs is described in pending U.S. application Ser. No. 10/213,477, filed Aug. 7, 2002 (inventors Rosalyn Ritts and Hoi-Cheong Steve Sun; assignee PowerZyme, Inc.), the text of which is incorporated by reference.

In general, however, both biocompatible membranes and PTMs associated with polypeptides which can be used in an analyte sensor of the present invention include the following steps:

1. Form a solution or suspension of synthetic polymer material in a solvent or mixed solvent system. The solution or suspension can be a mixture of two or more block copolymers, although it may contain one or more polymers and/or copolymers. The solution or suspension preferably contains 1 to 90% w/v synthetic polymer material, more preferably 2 to 70%, or yet more preferably 3 to 20% w/v. Seven % w/v is particularly preferred.

2. One or more polypeptides (typically with solubilizing detergent) are placed in solution or suspension, either separately or by being added to the existing polymer solution or suspension. Where the solvent used to solubilize the synthetic polymer materials is the same, or of similar characteristics and solubility to that which can solubilize the polypeptide, it is usually more convenient to add the polypeptide to the polymer solution or suspension directly. Otherwise, the two or more solutions or suspensions containing the synthetic polymer materials and the polypeptide must be mixed, possibly with an additional cosolvent or solubilizer. Most often, the solvent used for the polypeptide is aqueous.

Mixing of these solutions and/or suspensions is often a relatively simple matter and can be accomplished by hand or with automated mixing tools. Heating or cooling may also be useful in membrane formation depending on the solvents and polymers used. In general, rapidly evaporating solvents tend to form membranes better with cooling while extremely slowly evaporating solvents would most likely benefit from a slight degree of heating. One can examine the boiling point of solvents used to select those with the most favorable characteristics provided they are appropriate for the polymer used. One must, of course, however consider also the need to incorporate the polypeptide into the solvent polymer mixture, which can be a nontrivial matter. It is possible, for example, to mix 5 microliters of a detergent solubilized Complex I (0.15% w/v dodecyl maltoside) having 10 mg/ml of Complex I into 95 microliters of a mixture of a 3.2% w/v polystyrene-polybutadiene-polystyrene triblock copolymer (a completely hydrophobic triblock Sold under the trademark STYROLUX 3G55, available from BASF) in a 50/50 mixture of acetone and hexane and to deposit same in a manner that will allow for membrane formation. In this case, the final mixture includes about 5% v/v of water, and 0.75% w/w Complex I relative to the weight of the synthetic polymer material. Generally, the solutions are sufficiently stable at room temperature to be useful for at least about 30 minutes, provided that the solvents do not evaporate during that time. They also can be stored overnight, or longer, generally under refrigerated conditions.

3. A volume of the final solution or suspension including both the polypeptide(s) and the synthetic polymer materials is formed into a membrane and allowed to at least partially dry, thereby removing at least a portion of the solvent. It is possible to completely dry some of the membranes produced in accordance with the invention or to substantially dry same. By substantially dry it is meant that there may be some residual solvent, up to about 15%, which is often retained even if left out at room temperature for several hours. Biocompatible membranes can be formed, as opposed to PTMs, by selecting polymers that do not have the observed proton-tunneling properties. In one embodiment, a biocompatible membrane can be formed and associated with a second membrane, which is a PTM-that is they form a multi-layered membrane. Preferably, the two membranes are placed into intimate contact, such as by being stacked. Otherwise, they may be spaced apart and the gap filled by some fluid, usually a liquid electrolyte.

In a particularly preferred embodiment, substantially all of the weight of the finished membrane will be either polypeptide or synthetic polymer material. The amount of synthetic polymer material, including additives and stabilizing polymers, if any, ranges from about 70% to about 99% by weight of the finished membrane. However, it may be desirable to have relatively high polypeptide content or it may be necessary to retain some solvent, so the amount of synthetic polymer material may be reduced accordingly. Generally, however, at least about 50% by weight of the finished biocompatible membrane will be synthetic polymer material. When the synthetic polymer material is a mixture that includes a block copolymer and a polymer or copolymer, other than a stabilizing polymer, the block copolymer can be present in an amount of at least about 35% by weight of the membrane. Up to about 30% by weight of the membrane can be “additives” and “other materials” (collectively) as defined herein. More preferably the amount of additives and other materials is up to about 15% by weight of the membrane. Up to about 30% by weight of the synthetic polymer material can be stabilizing polymer. Generally the stabilizing polymer will be present in an amount of between about 5 and about 20% of the weight of the synthetic polymer material used. These numbers are exclusive of microparticles and any wetting agent used to treat the surface of a PTM.

Identifying which solvents are particularly useful in accordance with the present invention and which combination of polymers and polypeptides and solvents should be used depends on a number of factors, some of which have already been discussed in terms of miscibility, evaporation and the like. The polymer and polypeptide constituents must be able to be completely dissolved in the solvent or solvent mixture. Evaporation rate must be sufficiently long to allow one time to produce a membrane. However, the amount of time should not be so long as to render manufacturing impractical. While nonpolar solvents may be useful, generally more nonpolar solvents may not be useful in certain circumstances as ionic or hydroxyl components of the polymer may be poorly soluble in completely nonpolar solvents. Thus one may be able to dissolve a highly rigid, hydrophobic component such as polystyrene and be unable to simultaneously dissolve a highly ionic component such as an acrylic acid. However, with polymers of completely hydrophobic character, then nonpolar solvents are preferred. The solvents should generally be, in part, nonaqueous as the polymer should be at least in part nonwater dissolvable. And while water-miscibility is most desired for membrane protein reconstitution, it is not a rigidly limiting factor. Thus, preferably, all solvents are nonaqueous. The solvent for the polypeptide and stabilizing polymers, however, is predominantly water or at least water miscible.

Preferred methods of forming biocompatible membranes including both at least one synthetic polymer material and a stabilizing polymer include the step of making an appropriate solution of block copolymer and, usually separately stabilizing polymer and polypeptide. As described elsewhere, the polypeptide may include one or more detergents or surfactants and is typically in an aqueous solution. Once the appropriate solutions are made and mixed, membranes can be made by any of the techniques disclosed herein or known to the art including, for example, coating a perforated dielectric substrate with the solution followed by at least partial evaporation of solvents. Such evaporation can be facilitated in a vacuum.

One method of forming a biocompatible membrane, including a hydrogen-bonding rich stabilizing polymer, is as follows:

1. A solution or suspension of Protolyte A700 block copolymer in a solvent as supplied is diluted with an equal volume of ethanol (5% water w/v). The solution contains about 5% w/v of block copolymer.

2. Separately, an aqueous solution or suspension of the stabilizing agent is made by mixing 943 mg of polyethylene glycol (PEG) 8000 to produce a solution having a concentration of about 2.3% w/v. The concentration of the stabilizing agent in solution is near the saturation limit.

3. Next, 4 microliters of a solution including 10 mg/ml of E. coli derived Complex I along with 0.15% w/v of dodecyl maltoside is added to 6 microliters of the PEG solution and mixed them to generate a solution or suspension.

4. The 10 microliters of the solution is then mixed with 10 microliters of the solution including the block copolymer.

5. A small volume (e.g., 4 microliters) resulting solution is dropped onto the apertures of a subset of apertures (holes drilled through the support) of a perforated substrate of 1 mil (25.4 microns) thick KAPTON, a brand of polyimide, having apertures that are 100 micrometers in diameter and 1 mil deep.

6. The solution is allowed to air dry in a hood thereby removing the solvent.

7. Steps 5 and 6 are repeated as needed to cover all apertures.

The above-described method of introducing polypeptide to a solution containing a stabilizing polymer prior to mixing with non-aqueous solvent(s) in the presence of block copolymers is believed to stabilize the function of polypeptides used in the biocompatible membrane. However, the polymer and block copolymer could also be mixed and the resulting solution mixed with a generally aqueous polypeptide solution. Optionally one would check each aperture to ensure membrane formation, or check at least a statistically relevant number of apertures microscopically. If apertures do not contain a membrane, holes are repaired using additional solution and a micropipette-scaled pipetting device. It typically requires only a very small volume of solution to repair such holes. The membranes can be completely or substantially completely dried in a vacuum apparatus, or desiccator. Membranes so formed may be stored dried in vacuum or desiccated, if desired.

Parameters can be adjusted depending on such conditions as the membrane material, the size of biocompatible membrane, the thickness of the biocompatible membrane, the structure of the support, and the like.

Once the polypeptide/synthetic polymer material solution has been produced, it can be formed into a membrane. Biocompatible membranes in accordance with the present invention can be free standing membranes. Such membranes can be formed by pouring the solution into a pan or onto a sheet such that they achieve the desired thickness. Once the solution has been dried and the solvent dried off, the dry membrane may be removed from the pan or peeled from the backing layer. Suitable antitack agents may be used to assist in this process. Biocompatible membranes can be formed against a solid material, such as by coating onto glass, carbon that is surface modified to increase hydrophobicity, or a polymer (such as polyvinyl acetate, PDMS, Kapton®, a perfluorinated polymer, PVDF, PEEK, polyester, or UHMWPE, polypropylene or polysulfone). Polymers such as PDMS provide an excellent support that can be used to establish openings on which biocompatible membranes can be formed.

The membrane may then be cut or shaped as needed or used as is. Furthermore, to facilitate use of the membrane, it may be attached physically or through a fastening device or adhesive to a holder if desired. This can be conceptualized as stretching a canvas over a frame prior to painting a picture when the frame is the support and the membrane is the canvas. Alternatively, the membrane may be formed directly or in connection with such a structure. A suitable analogy would be taking a child's bubble wand, used for blowing bubbles, and dipping it into a solution of soap and water. A film of soap and water forms across the opening of the wand. The structural material at the periphery of the film allows the film to be handled and manipulated and provides rigidity and strength. It also helps provide the desired shape of the film. An analogous process can be employed using a physical structure and the membrane-forming solutions of the present invention.

Biocompatible membranes used in the invention are optionally stabilized against a solid support. One method for accomplishing such stabilization uses sulfur-mediated linkages of lipid-related molecules to glue, tether or bond metal surfaces or surfaces of another solid support to biocompatible membranes. For example, a porous support can be coated with a sacrificial or removable filler layer, and the coated surface smoothed by, for example, polishing. Such a porous support can include any of the proton-conductive polymeric membranes discussed, typically so long as the proton-conductive polymeric membrane can be smoothed following coating, and is stable to the processing described below. One useful porous support is glass frit. The smoothed surface is then coated (with prior cleaning as necessary) with metal, such as with a first layer of chrome and an overcoat of gold. The sacrificial material is then removed, such as by dissolution, taking with it the metallization over the pores but leaving a metallized surface surrounding the pores. The sacrificial layer can comprise photoresist, paraffin, cellulose resins (such as ethyl cellulose), and the like.

The tether or glue comprises alkyl thiol, alkyl disulfides, thiolipids and the like adapted to tether a biocompatible membrane. Such tethers are described for example in Lang et al., Langmuir 10: 197-210, 1994. Additional tethers of this type are described in Lang et al., U.S. Pat. No. 5,756,355 and Hui et al., U.S. Pat. No. 5,919,576.

The biocompatible membrane can be formed across the pores, perforations or apertures and polypeptide incorporated therein by, for example, the methods described in detail in Niki et al., U.S. Pat. No. 4,541,908 (annealing cytochrome C to an electrode) and Persson et al., J. Electroanalytical Chem. 292: 115, 1990. Such methods can comprise the steps of: making an appropriate solution of polypeptide and synthetic polymer material as previously discussed, the perforated substrate preferably a dielectric substrate is dipped into the solution to form the polypeptide-containing biocompatible membranes. Sonication or detergent dilution may be required to facilitate enzyme incorporation into a biocompatible membrane. See, for example, Singer, Biochemical Pharmacology 31: 527-534, 1982; Madden, “Current concepts in membrane protein reconstitution,” Chem. Phys. Lipids 40: 207-222, 1986; Montal et al., “Functional reassembly of membrane proteins in planar lipid bilayers,” Quart. Rev. Biophys. 14: 1-79, 1981; Helenius et al., “Asymmetric and symmetric membrane reconstitution by detergent elimination,” Eur. J. Biochem. 116: 27-31, 1981; Volumes on membranes (e.g., Fleischer and Packer (eds.)), in Methods in Enzymology series, Academic Press.

Alternatively, a thin partition made (preferably but not necessarily) of a hydrophobic material such as Teflon with a small aperture has a small amount of amphiphile introduced. The coated aperture is immersed in a dilute electrolyte solution upon which the droplet will thin and spontaneously self-orient spanning the-aperture. Biocompatible membranes of substantial area have been prepared using this general technique. Two common methods for formation of the biocompatible membranes themselves are the Langmuir-Blodgett technique and the injection technique. Referred to earlier and described in detail in copending U.S. application Ser. No. 10/213,530, incorporated herein by reference to the extent permitted.

The thickness of a substrate, be it a perforated substrate having apertures or a porous material, could be, for example, about 15 micrometers to about 5 millimeters, preferably about 15 to about 1,000 micrometers, and more preferably, about 15 micrometer to about 30 micrometers. The width of the perforations or pores is, for example, about 1 micrometer to about 1,500 micrometers, more preferably about 20 to about 200 micrometers, and even more preferably, about 60 to about 140 micrometers. About 100 micrometers is particularly preferred. Preferably, perforations or pores comprise in excess of about 30% of the area of any area of the dielectric substrate involved in transport between the chambers, such as from about 50 to about 75% of the area.

The thickness of the biocompatible membrane in accordance with the present invention can be adjusted by known techniques such as controlling the volume introduced to a particular size pore, perforation, pan or tray, etc. The thickness of the membrane will be dictated largely by its composition and function. A membrane intended to include a transmembrane proton transporting complex such as complex I must be thick enough to provide sufficient support and orientation to the polypeptide complex. It should not, however, be so thick as to prevent effective transportation of the proton across the membrane. For an aperture or perforation of about 100 microns in diameter in an array of about 100 apertures and a solution including complex I in an amount of about 4 microliters in a copolymer solution containing about 7% w/v of the poly(2-methyloxazoline)-block-poly(dimethylsiloxane)-block-poly(2-methyloxazoline)-triblock copolymer described in one of the previously identified Meier et al. articles, a membrane of suitable thickness can be obtained. The thickness of the membrane can vary widely depending upon its needed longevity, its function, etc. Membranes that are designed to transport protons for example are often thinner than membranes that are associated with a polypeptide involved in an oxidation reaction. However, in general, the membranes will range from between about 10 nanometers to 100 micrometers or even thicker. Indeed, biocompatible membranes useful for transporting protons in an analyte sensor have been successful at thicknesses of 10 nanometers up to 10 micrometers. Again, thicker membranes are possible.

Operation of One Embodiment

In a preferred embodiment of an analyte sensor of the present invention, the sensor comprises a first compartment adapted for the introduction of an analyte, and a second compartment separated from the first compartment by a barrier comprising a biocompatible membrane associated with a polypeptide, the polypeptide capable in participating in the transport of a species, in this case a proton, across the barrier. A detector comprising a pair of electrodes is used to detect the transported species, a first electrode being disposed in an anode compartment, and a second electrode being disposed in a cathode compartment.

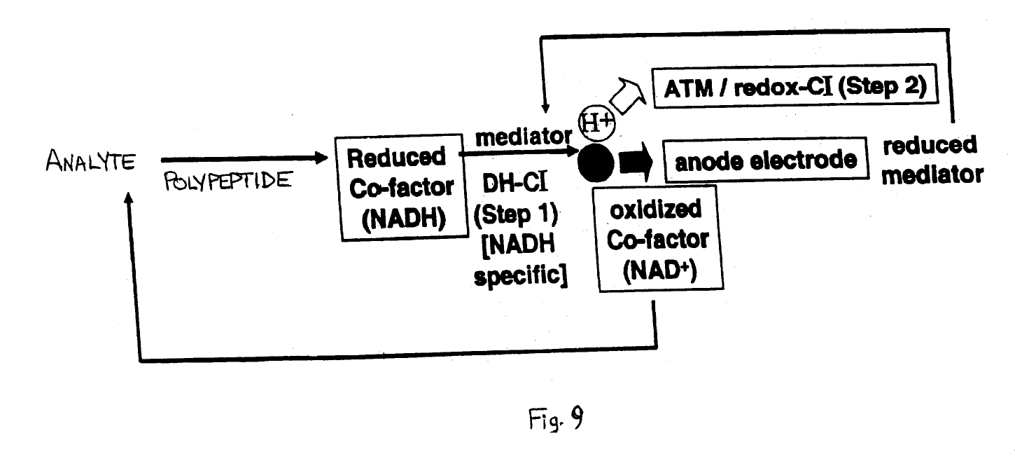

FIG. 9 shows a schematic block diagram representation the anode side in accordance with this embodiment. The anode compartment is the first compartment, to which is introduced a solution potentially containing the analyte, methanol. In this case, the targeted analyte is an organic molecule that is consumed, being transformed into a single carbon molecule, its consumption generating protons. Examples of other compounds the sensor could be adapted to detect include, without limitation, oxidizable sugars and sugar alcohols, alcohols, organic acids such as pyruvates, succinates, etc., fatty acids, lactic acids, citric acid, etc., amino acids and short polypeptides, aldehydes, ketones, etc. The analyte in this embodiment, methanol, is first acted upon by a polypeptide, in this case an alcohol dehydrogenase, designated DH. As will be seen below, other dehydrogenases such as aldehyde dehydrogenase and formate dehydrogenase can also be used or can be used in conjunction with one another. These polypeptides are enzymes which are capable of acting upon an analyte to generate electrons and protons.

The protons and electrons are transferred by the coordinated action of the polypeptides on the analyte to an electron carrier also referred to as a cofactor. One such electron carrier is NAD+/NADH. Electron carriers, when present, generally are provided in concentrations of between about 1 microMolar to about 2 Molar, more preferably about 10 microMolar to about 1 Molar, and most preferably about 100 microMolar to about 500 milliMolar.

Under the influence of the polypeptides, protons and electrons, NAD⁺ is converted to NADH. From this point, electrons and/or protons can be handed off and traded between a number of additional cofactors and/or transfer mediators. The electron transfer mediator facilitates transfer of electrons released from the electron carrier to another molecule, in this case, and typically, an electrode. Examples, in addition to those previously identified, include phenazine methosulfate (PMS), pyrroloquinoline quinone (PQQ, also called methoxatin), Hydroquinone, methoxyphenol, ethoxyphenol, or other typical quinone molecules, methyl viologen, 1,1′-dibenzyl-4,4′-dipyridinium dichloride (benzyl viologen), N,N,N′,N′-tetramethylphenylenediamine (TMPD) and dicyclopentadienyliron (C₁₀H₁₀Fe, ferrocene). Electron transfer mediators, when present, generally are provided in concentrations of between about 1 microMolar to abut 2 Molar, more preferably between about 10 microMolar and about 2 Molar, and even more preferably between but 100 microMolar and about 2 Molar.

For simplicity, however, and as illustrated in FIG. 9, the reduced cofactor or electron carrier can next interact with the polypeptide, in this case, the dehydrogenase DH function of Complex I (CI) embedded in a biocompatible membrane in accordance with the present invention. The Complex I (CI) liberates protons from the NADH molecule, as well as electrons. The electrons might flow directly to the anode. However, more often, they are taken up by a transfer mediator, which then transports the electrons to the anode. ATM refers to Active Transport Membrane.

NADH dehydrogenase Complex I is an interesting polypeptide in that it also can participate in transporting protons across the biocompatible membrane. What is particularly interesting, however, is that the protons transferred are not necessarily the protons liberated by the action of the dehydrogenase portion of Complex I. Therefore, to be most successful, an analyte sensor in accordance with this particular aspect of the invention will contain additional proton species in the anode compartment. The proton transporting function of Complex I is illustrated in FIG. 9, as the redox function.

When the transfer mediator gives up its electrons to the anode, it has been oxidized, allowing it to be capable of obtaining additional electrons liberated by oxidizing other electron carriers. Oxidized cofactor (NAD⁺) is also now ready to receive protons and electrons upon interaction, of polypeptide with the analyte. The reactions just described occur at the anode electrode and in the anode compartment and can be exemplified chemically as follows:

H₂O+NADH [symbol] NAD⁺+H₃O⁺+2e⁻

This reaction can be fed by the following reactions:

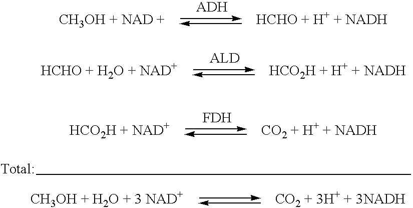

Thus, the reactions and the electron-generating reaction sum as follows:

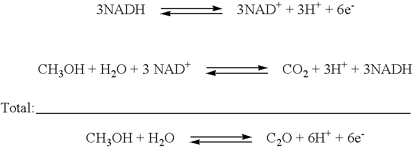

The polypeptide that can be used to generate a reduced electron carrier (such as NADH as illustrated above) from an organic molecule such as methanol can start with a form of alcohol dehydrogenase (ADH). Suitable ADH enzymes are described for example in Ammendola et al., “Thermostable NAD(+)-dependent alcohol dehydrogenase from Sulfolobus solfataricus: gene and protein sequence determination and relationship to other alcohol dehydrogenases,” Biochemistry 31: 12514-23, 1992; Cannio et al., “Cloning and overexpression in Escherichia coli of the genes encoding NAD-dependent alcohol dehydrogenase from two Sulfolobus species,” J. Bacteriol. 178: 301-5, 1996; Saliola et al., “Two genes encoding putative mitochondrial alcohol dehydrogenases are present in the yeast Kluyveromyces lactis,” Yeast 7: 391-400, 1991; and Young et al., “Isolation and DNA sequence of ADH3, a nuclear gene encoding the mitochondrial isozyme of alcohol dehydrogenase in Saccharomyces cerevisiae,” Mol. Cell Biol. 5: 3024-34, 1985. If the resulting formaldehyde is oxidized, an aldehyde dehydrogenase (ALD) is used. Suitable ALD enzymes are described for example in Peng et al., “cDNA cloning and characterization of a rice aldehyde dehydrogenase induced by incompatible blast fungus,” GeneBank Accession AF323586; Sakano et al., “Arabidopsis thaliana [thale cress] aldehyde dehydrogenase (NAD+)-like protein” GeneBank Accession AF327426. If the further resulting formic acid is oxidized, a formate dehydrogenase (FDH) is used. Suitable FDH enzymes are described for example in Colas des Francs-Small, et al., “Identification of a major soluble protein in mitochondria from nonphotosynthetic tissues as NAD-dependent formate dehydrogenase [from potato],” Plant Physiol. 102(4): 1171-1177, 1993 ; Hourton-Cabassa, “Evidence for multiple copies of formate dehydrogenase genes in plants: isolation of three potato fdh genes, fdh1, fdh2, and fdh3, ” Plant Physiol. 117: 719-719, 1998.

For reasons discussed below, it can be useful to use polypeptides that are adapted to use or otherwise can accommodate quinone-based electron carriers. Such polypeptides are, for example, described in: Pommier et al., “A second phenazine methosulphate-linked formate dehydrogenase isoenzyme in Escherichia coli,” Biochim Biophys Acta. 1107(2): 305-13, 1992. (“The diversity of reactions involving formate dehydrogenases is apparent in the structures of electron acceptors which include pyridine nucleotides, 5-deazaflavin, quinones, and ferredoxin”); Ferry, J. G. “Formate dehydrogenase” FEMS Microbiol. Rev. 7(3-4): 377-82, 1990. (formaldehyde dehydrogenase with quinone activity); Klein et al., “A novel dye-linked formaldehyde dehydrogenase with some properties indicating the presence of a protein-bound redox-active quinone cofactor” Biochem J. 301 (Pt 1): 289-95, 1994. (representative of a number of articles on dehydrogenases with bound quinone cofactors); Goodwin et al., “The biochemistry, physiology and genetics of PQQ and PQQ-containing enzymes” Adv. Microb. Physiol. 40:1-80, 1998. (on alcohol dehydrogenases that utilize quinones); Maskos et al., “Mechanism of p-nitrosophenol reduction catalyzed by horse liver and human pi-alcohol dehydrogenase (ADH)” J. Biol. Chem. 269(50): 31579-84, 1994 (example of mediator-catalyzed transfer of electrons from NADH to an electrode following NADH reduction by an enzyme); and Pandey, “Tetracyanoquinodimethane-mediated flow injection analysis electrochemical sensor for NADH coupled with dehydrogenase enzymes” Anal. Biochem. 221(2): 392-6, 1994.

The corresponding reaction at the cathode in the cathode compartment (second compartment) can be any reaction that consumes the produced electrons with a useful redox potential. Using oxygen, for example, the reaction can be:

2H₃O⁺+½O₂+2e⁻Z,2 3H₂O

Using reaction 2, the catholyte solution (an electrolyte used in the cathode compartment) can be buffered to account for the consumption of hydrogen ions, hydrogen ion donating compounds can be supplied during operation of the analyte sensor, or more preferably, the barrier between the anode and cathode compartments is sufficiently effective to deliver the neutralizing hydrogen ions (hydrogen ion or proton).

In one embodiment, the corresponding reaction at the cathode is:

H₂O₂+2H⁺+2e⁻⇄2H₂O

The cathode reactions result in a net production of water, which, if significant, can be dealt with by, for example, providing for space for overflow liquid, or providing for vapor-phase exhaust. A number of electron acceptor molecules are often solids at operating temperatures or solutes in a carrier liquid, in which case the cathode chamber should be adapted to carry such non-gaseous material.

Separation Devices

In yet another embodiment of the present invention is provided a detection device including a separation module adapted to separate a sample into at least two sample components. Such separation can be accomplished, for example, by a chromatographic, electrophoretic, charge-flow (wherein an electrical or magnetic field influences migration of species within a fluid stream), ion pulse device generating acoustic waves to influence migration, a device that induces pH changes (for instance with light or ampholytes) with consequent discrimination in migration rates, centrifuge that separates on density or rate of sedimentation (and, for example, from which device the separated layers are drained past the analyte sensor after centrifugation), cell sorter, and any other device for separating chemical or cellular species. The results of the separation, at least one component, will then be transferred or directed by a transfer element, e.g. tubing and a pump, into a first compartment of an analyte sensor of the present invention adapted to detect a particular analyte.

For cellular detections (in this or other contexts for use of the sensor), the species detected can be an enzymatic product of the cells, or an enzymatic product produced by enzymes associated with the cells with antibodies or another affinity association.

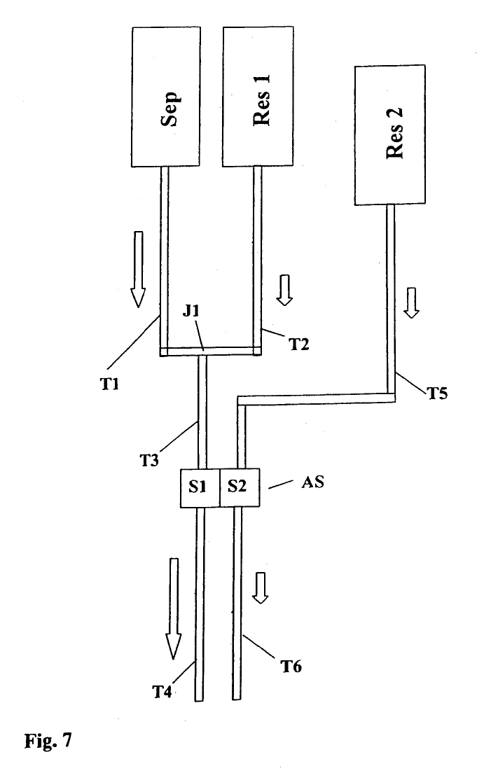

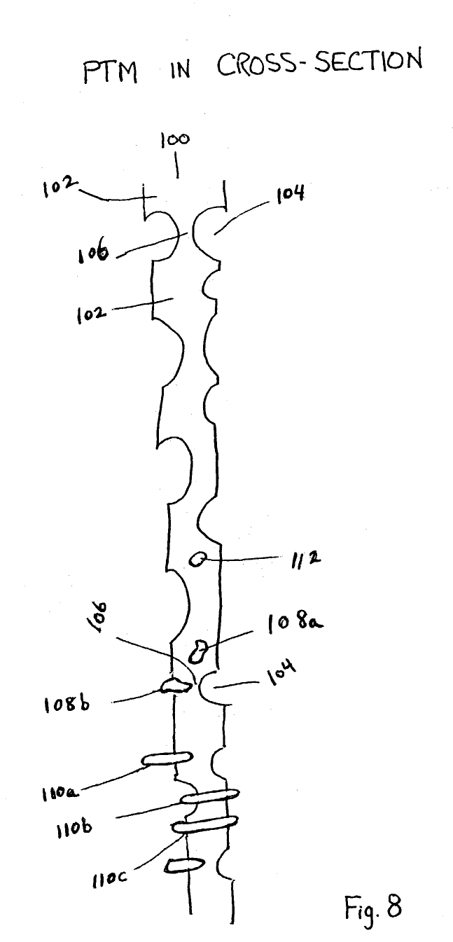

In flow detections, it will sometimes be useful to have concurrent flow of supportive reagents. In one case, the supportive reagents add one or more components needed and not provided in the stream from the separation device. On the other side of the biocompatible membrane from that into which the stream from the separation device flows, concurrent flow can be used, if needed, to assure a fresh supply of any detection supportive components. For example, as illustrated in FIG. 7, the separation device Sep feeds a fluid flow into tubing T1 (indicated by the adjacent arrow), and reservoir Res 1 provides reagents (if needed) for detection in the analyte sensor AS (having sides S1 and S2) through tubing T2 (flow indicated by the adjacent arrow). The flows through tubing T1 and T2 are joined at junction J1, which can include a mixer, e.g., a static mixer, the merged flow proceeds to side S1 of the analyte sensor AS, and then out through tubing T4. Junction J1 can placed closer or farther from the analyte sensor as appropriate to support any chemistries (e.g., reaction kinetics) required by the fluid from reservoir Res 1. Reservoir Res 2 provides reagents, if needed, through tubing T5 to side S2, then out through tubing T6.

It will be apparent that the same type of supportive reagent flows can be used where the sample is injected without separation (e.g., where item Sep is replaced with an injector). Data or read-out is then taken from the analyte sensor S1/S2.

It will also be apparent that the separation module can perform more than one separation prior to the transfer of a component to the analyte sensor. The separation module may be adapted to subject the sample to a series of separations, which may be accomplished using more than one type of separation device. For example, a cell sorter may be employed, followed by centrifugation, or multiple centrifugations. In yet another example, a sample may be subject to centrifugation followed by separation by a chromatography column.

### EXAMPLES NOS. 1-436

(Biocompatible Membranes)

### EXAMPLE NO. 1

A solution useful for producing a biocompatible membrane in accordance with the present invention was produced as follows: 7% w/v (70 mg) of a block copolymer (poly (2-methyloxazoline)-polydimethyl siloxane-poly(2-methyl(oxazoline) having an average molecular weight of 2KD-5KD-2KD was dissolved in an 95% v/v A 5% v/v ethanol/water solvent mixture with stirring using a magnetic stirrer. Six microliters of this solution was removed and mixed with four microliters of a solution containing 0.015% w/v dodecyl maltoside, 40 micrograms of Complex I (10 mg/ml) in water. This is then mixed. The resulting solution contains 4.2% w/v polymer, 55% EtOH v/v, 45% H2O v/v, 0.06% w/v dodecyl maltoside and protein/polymer ratio is 6% w/w.

### EXAMPLE NO. 2

A solution useful for producing a biocompatible membrane in accordance with the present invention was prepared generally as described in Example No. 1 with the following changes: less polypeptide solution was used so as to provide a final solution including 0.015% w/v dodecyl maltoside and 1.5% w/w polypeptide relative to synthetic polymer materials.

### EXAMPLE NO. 3

A solution useful for producing a biocompatible membrane in accordance with the present invention was prepared generally as described in Example No. 1 with the following changes: less polypeptide solution was used so as to provide a final solution including 0.03% w/v dodecyl maltoside and the final solution contained 3.0% w/w polypeptide relative to synthetic polymer materials.

### EXAMPLE NO. 4

A solution useful for producing a biocompatible membrane in accordance with the present invention was prepared generally as described in Example No. 1 with the following changes: less polypeptide solution was used so as to provide a final solution including 0.045 w/v dodecyl maltoside and the final solution contained 4.5% w/w polypeptide relative to synthetic polymer materials.

### EXAMPLE NO. 5

A solution useful for producing a biocompatible membrane in accordance with the present invention was prepared generally as described in Example No. 1 with the following changes: less polypeptide solution was used so as to provide a final solution including 0.0075 w/v dodecyl maltoside and the final solution contained 0.75% w/w polypeptide relative to synthetic polymer materials.

### EXAMPLE NO. 6

A solution useful for producing a biocompatible membrane in accordance with the present invention was prepared generally as described in Example No. 5 with the following changes: the synthetic polymer material was originally present in a solution of 5.0% w/v. Sufficient polypeptide solution of the type described in Example 1 was added so as to produce a final solution including 0.0075% w/v dodecyl maltoside and 0.75% w/w polypeptide relative to synthetic polymer materials.

### EXAMPLE NO. 7

A solution useful for producing a biocompatible membrane in accordance with the present invention was prepared generally of the type described in Example No. 6 with the following changes: sufficient polypeptide solution as described in Example 1 was included so as to produce a final solution including 0.015% w/v dodecyl maltoside and the final solution contained 1.5% w/w polypeptide relative to synthetic polymer materials.

### EXAMPLE NO. 8

A solution useful for producing a biocompatible membrane in accordance with the present invention was prepared generally of the type described in Example No. 6 with the following changes: sufficient polypeptide solution as described in Example 1 was included so as to produce a final solution including 0.03% w/v dodecyl maltoside and the final solution contained 3% w/w polypeptide relative to synthetic polymer materials.

### EXAMPLE NO. 9

A solution useful for producing a biocompatible membrane in accordance with the present invention was prepared generally of the type described in Example No. 6 with the following changes: sufficient polypeptide solution as described in Example 1 was included so Has to produce a final solution including 0.045% w/v dodecyl maltoside and the final solution contained 4.5% w/w polypeptide relative to synthetic polymer materials.

### EXAMPLE NO. 10

A solution useful for producing a biocompatible membrane in accordance with the present invention was prepared generally of the type described in Example No. 6 with the following changes: sufficient polypeptide solution as described in Example 1 was included so as to produce a final solution including 0.06% w/v dodecyl maltoside and the final solution contained 6.0% w/w polypeptide relative to synthetic polymer materials.

### EXAMPLES NOS. 11-15

Solutions useful for producing a biocompatible membrane in accordance with the present invention were prepared generally as described in Example Nos. 1-5 respectively except that the amount of the synthetic polymer material used in each solution was originally 10% w/v. When 6 microliters of that solution was mixed with sufficient polypeptide solution of the type described in Example 1 a final solution was produced including 0.06, 0.15, 0.03, 0.045 and 0.0075% w/v dodecyl maltoside and 6.0, 1.5, 3.0, 4.5 and 0.75% w/w polypeptide relative to synthetic polymer materials, respectively.

### EXAMPLE NO. 16

A solution useful for producing a biocompatible membrane in accordance with the present invention was prepared generally as described in Example No. 3, however, the solvent used to dissolve the synthetic polymer material included ethanol, 25% methanol v/v and the amount of water indicated in Example No. 3. Sufficient polypeptide solution was used so as to provide a final solution including 0.03% w/v dodecyl maltoside and 3.0% w/w polypeptide relative to synthetic polymer materials.

### EXAMPLE NO. 17

A solution useful for producing a biocompatible membrane in accordance with the present invention was prepared generally as described in Example No. 2, however, the solvent used to dissolve the synthetic polymer material included 47.5% v/v ethanol, 2.5% v/v water, 25% v/v Tetrahydrofuran (“THF”), 25% v/v dichloromethane. Sufficient polypeptide solution was used so as to provide a final solution including 0.015. % w/v dodecyl maltoside and 1.5% w/w polypeptide relative to synthetic polymer materials.

### EXAMPLE NO. 18

A solution useful for producing a biocompatible membrane in accordance with the present invention can be prepared generally as described in Example No. 6, however, the solvent used to dissolve the synthetic polymer material included 9.5% v/v ethanol, 0.5% v/v water, 40% v/v acetone, and 40% v/v hexane.

### EXAMPLE NOS. 19-24

Solutions useful for producing a biocompatible membrane in accordance with the present invention were prepared generally as described in Example Nos. 11-15 above, however, the final concentration of dodecyl maltoside was 0.15% w/v. Solutions useful for producing a biocompatible membrane in accordance with the present invention can be prepared generally as described in Example No. 4 above, however, the balance of the surfactant used in the polypeptide solution is dodecyl b-D-glucopyranoside and the final concentration of the surfactants is 0.15% w/v.

### EXAMPLE NO. 26

A solution useful for producing a biocompatible membrane in accordance with the present invention was prepared generally as described in Example No. 9 above, however, the surfactant used in the polypeptide solution included a mixture of a polymeric surfactant sold under the trademark PLURONIC L101, lot WPDX-522B from BASF, Ludwigshafen Germany and the same concentration of dodecyl maltoside specified in Example No. 9. The polymeric surfactant was diluted to 0.1% v/v of its supplied concentration in the final solution.

### EXAMPLE NO. 27

A solution useful for producing a biocompatible membrane in accordance with the present invention was prepared generally as described in Example No. 2 above, however the surfactant used in the polypeptide solution included a mixture of a polymeric surfactant sold under the trademark DISPERPLAST, lot no. 31J022 from BYK Chemie, Wallingford Conn. and the same concentration of dodecyl maltoside specified in Example No. 2. The polymeric surfactant was diluted to 0.135%v/v of the supplied concentration in the final solution.

### EXAMPLES NOS. 28-32

Solutions useful for producing a biocompatible membrane in accordance with the present invention can be prepared generally as described in Example Nos. 6-10 respectively, however, the synthetic polymer material used can be a poly(2-methyloxazoline)-polydimethylsiloxane-poly(2-methyloxazoline) (5% w/v) having an average molecular weight of 3 kD-7 kD-3 kD. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w pdlypeptide relative to synthetic polymer materials respectively.

### EXAMPLES NOS. 33-38

Solutions useful for producing a biocompatible membrane in accordance with the present invention were prepared generally as described in Example Nos. 1-5 respectively, however, the synthetic polymer material used was a mixture of two block copolymers, both of which were poly(2-methyloxazoline)-polydimethylsiloxane-poly(2-methyloxazoline), (total 7% w/v) one of which having an average molecular weight of 2 kD-5 kD-2 kD and the other 1 kD-2 kD-1 kD and the ratio of the first block copolymer to the second was about 67% to 33% of the total polymer used w/w. When 6 microliters of that solution was mixed with sufficient polypeptide solution of the type described in Example 1 a final solution was produced including 0.06, 0.015, 0.030, 0.045 and 0.0075% w/v dodecyl maltoside and 0.75, 1.5, 3.0, 4.5 and 6.0% w/w polypeptide relative to synthetic polymer materials respectively.

### EXAMPLES NOS. 39-43

Solutions useful for producing a biocompatible membrane in accordance with the present invention can be prepared generally as described in Example Nos. 11-15 respectively, however, the synthetic polymer material used can be a mixture of two block copolymers, both of which are poly(2-methyloxazoline)-polydimethylsiloxane-poly(2-methyloxazoline), (10% w/v) one of which having an average molecular weight of 1 kD-2 kD-1 kD and the other 3 kD-7 kD-3 kD and the ratio of the first block copolymer to the second being about 33% to 67% of the total polymer used w/w. When 6 microliters of that solution is mixed with sufficient polypeptide solution of the type described in Example 1 a final solution is produced including 0.075, 0.15, 0.30, 0.45 and 0.60% w/v dodecyl maltoside and 0.75, 1.5, 3.0, 4.5 and 6.0% w/w polypeptide relative to synthetic polymer materials respectively.

### EXAMPLES NOS. 44-48

Solutions useful for producing a biocompatible membrane in accordance with the present invention can be prepared generally as described in Examples Nos. 6-10 respectively, however, the synthetic polymer material used can be a mixture of two block copolymers, both of which are poly(2-methyloxazoline)-polydimethylsiloxane-poly(2-methyloxazoline), (5% w/v) one of which having an average molecular weight of 2 kD-5 kD-2 kD and the other 3 kD-7 kD-3 kD and the ratio of the first block copolymer to the second being about 33% to 67% of the total polymer used w/w. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 49-53

Solutions useful for producing a biocompatible membrane in accordance with the present invention can be prepared generally as described in Example Nos. 1-5 respectively, however, the synthetic polymer material used can be a mixture of two block copolymers, both of which are poly(2-methyloxazoline)-polydimethylsiloxane-poly(2-methyloxazoline), (7% w/v) one of which having an average molecular weight of 2 kD-5 kD-2 kD and the other 3 kD-7 kD-3 kD and the ratio of the first block copolymer to the second being about 67% to 33% of the total polymer used w/w. When 6 microliters of that solution is mixed with sufficient polypeptide solution of the type described in Example 1 a final solution is produced including 0.06, 0.015, 0.030, 0.045 and 0.0075% w/v dodecyl maltoside and 6.0, 1.5, 3.0, 4.5 and 0.025% w/w polypeptide relative to synthetic polymer materials respectively.

### EXAMPLES NOS. 54-58

Solutions useful for producing a biocompatible membrane in accordance with the present invention can be prepared generally as described in Examples Nos. 1-5 respectively, however, the synthetic polymer used can be a mixture of poly(2-methyloxazoline)-polydimethylsiloxane-poly(2-methyloxazoline) (7% w/v) having an average molecular weight of 2 kD-5 kD-2 kD in a solvent of 95% ethanol, 5% water mixed with a solution of 23.5% w/v polyethyleneglycol with an average molecular weight of approximately 3,300 Daltons in water in the proportion of 85% triblock copolymer solution, 15% polyethyleneglycol solution v/v. When 6 microliters of that solution is mixed with sufficient polypeptide solution of the type described in Example 1 a final solution is produced including 0.06, 0.015, 0.030, 0.045 and 0.0075% w/v dodecyl maltoside and 6.0, 1.5, 3.0, 4.5 and 0.75% w/w polypeptide relative to synthetic polymer materials respectively.

### EXAMPLES NOS. 59-63

A solution useful for producing a biocompatible membrane in accordance with the present invention was prepared generally as described in Example No. 12, however, the synthetic polymer used was a mixture of 10% w/v of poly(2-methyloxazoline)-polydimethylsiloxane-poly(2-methyloxazoline) having an average molecular weight of 2 kD-5 kD-2 kD in a solvent of 95% ethanol, 5% water mixed with a solution of 23.5% w/v polyethyleneglycol with an average molecular weight of approximately 8,000 Daltons in water in the proportion of 85% triblock copolymer solution, 15% polyethyleneglycol solution v/v. When 6 microliters of that solution was mixed with sufficient polypeptide solution of the type described in Example 1 a final solution was produced including 0.15% w/v dodecyl maltoside and 1.5% w/w polypeptide relative to synthetic polymer materials. Similar solutions can be made using the procedures of examples 11 and 13-15.

### EXAMPLES NOS. 64-68

Solutions useful for producing a biocompatible membrane in accordance with the present invention can be prepared generally as described in Examples Nos. 28-32 respectively, however, the synthetic polymer used can be a mixture of 5% w/v of poly(2-methyloxazoline)-polydimethylsiloxane-poly(2-methyloxazoline) having an average molecular weight of 3 kD-7 kD-3 kD in a solvent of 95% ethanol, 5% water mixed with a solution of 23.5% w/v polyethyleneglycol with an average molecular weight of approximately 3,300 Daltons in water in the proportion of 85% triblock copolymer solution, 15% polyethyleneglycol solution v/v. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 69-73

Solutions useful for producing a biocompatible membrane in accordance with the present invention can be prepared generally as described in Examples Nos. 1-5 respectively, however, the synthetic polymer used can be a mixture of 7% w/v of poly(2-methyloxazoline)-polydimethylsiloxane-poly(2-methyloxazoline) having an average molecular weight of 3 kD-7 kD-3 kD in a solvent of 95% ethanol, 5% water mixed with a solution of 23.5% w/v polyethyleneglycol with an average molecular weight of approximately 8,000 Daltons in water in the proportion of 85% triblock copolymer solution, 15% polyethyleneglycol solution v/v. When 6 microliters of that solution is mixed with sufficient polypeptide solution of the type described in Example 1 a final solution is produced including 0.060, 0.015, 0.030, 0.045 and 0.0075% w/v dodecyl maltoside and 6.0, 1.5, 3.0, 4.5 and 0.75% w/w polypeptide relative to synthetic polymer materials respectively.

### EXAMPLES NOS. 74-78

Solutions useful for producing a biocompatible membrane in accordance with the present invention can be prepared generally as described in Examples Nos. 6-10 respectively, however the synthetic polymer used can be a mixture of 5% w/v of poly(2-methyloxazoline)-polydimethylsiloxane-poly(2-methyloxazoline) having an average molecular weight of 2 kD-5 kD-2 kD in a solvent of 50% v/v acetone, 50% v/v heptane mixed with a solution of 5% w/v polystyrene of about 250,000 in molecular weight in 50% v/v acetone, 50% v/v octane in the proportion of 80% v/v block copolymer, 20% v/v polystyrene. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 79-83

Solutions useful for producing a biocompatible membrane in accordance with the present invention can be prepared generally as described in Examples Nos. 1-5 respectively, however, the synthetic polymer used can be a mixture of 7% w/v of poly(2-methyloxazoline)-polydimethylsiloxane-poly(2-methyloxazoline) having an average molecular weight of 2 kD-5 kD-2 kD in a solvent of 95% ethanol, 5% water mixed with a solution of 5% w/v of polymethylmethacrylate-polydimethylsiloxane-polymethylmethacrylate having an average molecular weight of 4 kD-8 kD-4 kD in a solvent of 50% v/v THF, 50% v/v dichloromethane in the proportion of 66% v/v to 33% v/v, respectively. When 6 microliters of that solution is mixed with sufficient polypeptide solution of the type described in Example 1 a final solution is produced including 0.06, 0.015, 0.030, 0.045 and 0.0075% w/v dodecyl maltoside and 6.0, 1.5, 3.0, 4.5 and 0.075% w/w polypeptide relative to synthetic polymer materials respectively.

### EXAMPLES NOS. 84-88

Solutions useful for producing a biocompatible membrane in accordance with the present invention were prepared generally as described in Examples Nos. 11-15 respectively, however, the synthetic polymer material used was 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water. When 6 microliters of that solution was mixed with sufficient polypeptide solution of the type described in Example 1 a final solution was produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLE NO. 89

A solution useful for producing a biocompatible membrane in accordance with the present invention can be prepared generally as described in Example No. 84 however, the solvent used to dilute the synthetic polymer material can include 50% v/v Tetrahydrofuran (“THF”), 50% v/v dichloromethane.

### EXAMPLES NOS. 90-94

Solutions useful for producing a biocompatible membrane in accordance with the present invention were prepared generally as described in Examples Nos. 84-88 above, however, the final concentration of dodecyl maltoside was 0.15% w/v.

### EXAMPLE NO. 95

Solutions useful for producing a biocompatible membrane in accordance with the present invention can be prepared generally as described in Example No. 85 above, however, the surfactant used in the polypeptide solution can include a mixture of dodecyl b-D-glucopyranoside and dodecyl maltoside and the final concentration of the surfactants is 0.15% w/v.

### EXAMPLE NO. 96

A solution useful for producing a biocompatible membrane in accordance with the present invention was prepared generally as described in Example No. 87 above, however, the surfactant used in the polypeptide solution included a mixture of a polymeric surfactant sold under the trademark PLURONIC L101, lot WPDX-522B from BASF, Ludwigshafen Germany and the same concentration of dodecyl maltoside specified in Example No. 87. The polymeric surfactant was diluted to 0.1% v/v of its supplied concentration in the final solution.

### EXAMPLE NO. 97

A solution useful for producing a biocompatible membrane in accordance with the present invention was prepared generally as described in Example No. 88 above, however, the surfactant used in the polypeptide solution included a mixture of a polymeric surfactant sold under the trademark DISPERPLAST, lot no. 31J022 from BYK Chemie, Wallingford Conn. and the same concentration of dodecyl maltoside specified in Example No. 88. The final concentration of the polymeric surfactant was diluted to 0.135% v/v of the supplied concentration in the final solution.

### Examples No. 98-102

Solutions useful for producing a biocompatible membrane in accordance with the present invention were prepared generally as described in Examples Nos. 84-88 respectively, however, the synthetic polymer material used was a mixture of two block copolymers, one of which was 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water, the other of which was 5% w/v of poly(2-methyloxazoline)-polydimethylsiloxane-poly(2-methyloxazoline) having an average molecular weight of 2 kD-5 kD-2 kD and the ratio of the first block copolymer to the second was about 67% to 33% of the total polymer used w/w. When 6 microliters of that solution was mixed with sufficient polypeptide solution as described in Example 1 a final solution was produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 103-107

Solutions useful for producing a biocompatible membrane in accordance with the present invention were prepared generally as described in Examples Nos. 84-88 respectively, however, the synthetic polymer material used was a mixture of two block copolymers, one of which was 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water, the other of which was 5% w/v of poly(2-methyloxazoline)-polydimethylsiloxane-poly(2-methyloxazoline) having an average molecular weight of 2 kD-5 kD-2 kD and the ratio of the first block copolymer to the second was about 33% to 67% of the total polymer used w/w. When 6 microliters of that solution waslmixed with sufficient polypeptide solution as described in Example 1 a final solution was produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 108-112

Solutions useful for producing a biocompatible membrane in accordance with the present invention can be prepared generally as described in Examples Nos. 103-107 respectively, however, the synthetic polymer material used can be a mixture of two block copolymers, one of which is 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water, the other of which is 5% w/v of polymethylmethacrylate-polydimethylsiloxane-polymethylmethacrylate having an average molecular weight of 4 kD-8 kD-4 kD in a solvent mixture of 50% v/v THF, 50% v/v dichloromethane, the ratio of the first block copolymer to the second being about 67% to 33% of the total polymer used w/w. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 113-117

Solutions useful for producing a biocompatible membrane in accordance with the present invention can be prepared generally as described in Examples Nos. 103-107 respectively, however, the synthetic polymer material used can be a mixture of two block copolymers, one of which is 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water, the other of which is 5% w/v of polymethylmethacrylate-polydimethylsiloxane-polymethylmethacrylate having an average molecular weight of 4 kD-8 kD-4 kD in a solvent mixture of 50% v/v THF, 50% v/v dichloromethane, the ratio of the first block copolymer to the second being about 33% to 67% of the total polymer used w/w. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 118-122

Solutions useful for producing a biocompatible membrane in accordance with the present invention were prepared generally as described in Examples Nos. 84-88 respectively, however, the synthetic polymer material used was a mixture of 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water mixed with a solution of 23.5% w/v polyethyleneglycol with an average molecular weight of approximately 3,300 Daltons in water in the proportion of 85% triblock copolymer solution, 15% polyethyleneglycol solution v/v. When 6 microliters of that solution was mixed with sufficient polypeptide solution of the type described in Example 1 a final solution was produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 123-127

Solutions useful for producing a biocompatible membrane in accordance with the present invention were prepared generally as described in Examples Nos. 84-88 respectively, however, the synthetic polymer material used was a mixture of 10% w/v of sulfonated styrene/ethylene-butylene/sulfonated styrene, supplied as Protolyte® A700, lot number LC-29/60-011 by Dais Analytic, Odessa, Fla. in solvent as supplied, diluted 50% v/v with ethanol containing 5% v/v water mixed with a solution of 23.5% w/v polyethyleneglycol with an average molecular weight of approximately 8,000 Daltons in water in the proportion of 85% triblock copolymer solution, 15% polyethyleneglycol solution v/v. When 6 microliters of that solution was mixed with sufficient polypeptide solution of the type described in Example 1 a final solution was produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 128-132

Solutions useful for producing a biocompatible membrane in accordance with the present invention can be prepared generally as described in Examples Nos. 6-10 respectively, however, the synthetic polymer material used can be 5% w/v of polymethylmethacrylate-polydimethylsiloxane-polymethylmethacrylate having an average molecular weight of 4 kD-8 kD-4 kD in a solvent mixture of 50% v/v THF, 50% v/v dichloromethane. When 6 microliters of that solution is mixed with sufficient polypeptide solution of the type described in Example 1 a final solution is produced including 0.0075, 0.015, 0.030, 0.045 and 0.060% w/v dodecyl maltoside and 0.75, 1.5, 3.0, 4.5 and 6.0% w/w polypeptide relative to synthetic polymer materials respectively.

### EXAMPLES NOS. 133-134

Solutions useful for producing a biocompatible membrane in accordance with the present invention were prepared generally as described in Examples Nos. 6 and 7 respectively, however, the synthetic polymer material used was 3.2% w/v of polystyrene-polybutadiene-polystyrene, supplied as Stryolux® 3G55, lot 7453064P by BASF, Ludwigshafen Germany in a 50%/50% v/v mixture of acetone and hexane. When 6 microliters of that solution was mixed with sufficient polypeptide solution of the type described in Example 1 a final solution was produced including 0.0075 and 0.015% w/v dodecyl maltoside and 0.75 and 1.5% w/w polypeptide relative to synthetic polymer materials respectively.

### EXAMPLES NOS. 135-136

Solutions useful for producing a biocompatible membrane in accordance with the present invention were prepared generally as described in Examples Nos. 6 and 7 respectively, however, the synthetic polymer material used was 3.2% w/v of polystyrene-polybutadiene-polystyrene, supplied as Stryolux® 3G55, lot 7453064P by BASF, Ludwigshafen Germany in a 50%/50% v/v mixture of acetone and heptane. When 6 microliters of that solution was mixed with sufficient polypeptide solution of the type described in Example 1 a final solution was produced including 0.0075 and 0.015% w/v dodecyl maltoside and 0.75 and 1.5% w/w polypeptide relative to synthetic polymer materials respectively.

### EXAMPLE NOS. 137-138

Solutions useful for producing a biocompatible membrane in accordance with the present invention were prepared generally as described in Examples Nos. 135 and 136 respectively, however, the synthetic polymer material used was 5% w/v of polystyrene-polybutadiene-polystyrene, supplied as Stryolux® 3G55, lot 7453064P by BASF, Ludwigshafen Germany in a 50%/50% v/v mixture of acetone and heptane. When 6 microliters of that solution was mixed with sufficient polypeptide solution of the type described in Example 1 a final solution was produced including 0.0075 and 0.015% w/v dodecyl maltoside and 0.75 and 1.5% w/w polypeptide relative to synthetic polymer materials respectively.

### EXAMPLES NOS. 139-141

Solutions useful for producing a biocompatible membrane in accordance with the present invention can be prepared generally as described in Examples Nos. 6-8 respectively, however, the synthetic polymer material used can be a mixture of 5% w/v of polystyrene-polybutadiene-polystyrene, supplied as Stryolux® 3G55, lot 7453064P by BASF, Ludwigshafen Germany in a 50%/50% v/v mixture of acetone and hexane and 5% w/v poly(2-methyloxazoline)-polydimethylsiloxane-poly(2-methyloxazoline) having an average molecular weight of 2 kD-5 kD-2 kD in the same solvent in the proportion of about 80% v/v to 20% v/v, respectively. When 6 microliters of that solution is mixed with sufficient polypeptide solution of the type described in Example 1 a final solution is produced including 0.0075, 0.015, and 0.030% w/v dodecyl maltoside and 0.75, 1.5 and 3.0% w/w polypeptide relative to synthetic polymer materials respectively.

### EXAMPLES NOS. 142-145

Solutions useful for producing a biocompatible membrane in accordance with the present invention can be prepared generally as described in Examples Nos. 139-141 respectively, however, the synthetic polymer material used can be a mixture of 5% w/v of polystyrene-polybutadiene-polystyrene, supplied as Stryolux® 3G55, lot 7453064P by BASF, Ludwigshafen Germany in a 50%/50% v/v mixture of acetone and hexane and 5% w/v poly(2-methyloxazoline)-polydimethylsiloxane-poly(2-methyloxazoline) having an average molecular weight of 3 kD-7 kD-3 kD in the same solvent in the proportion of about 80 % v/v to 20% v/v, respectively. When 6 microliters of that solution is mixed with sufficient polypeptide solution of the type described in Example 1 a final solution is produced including 0.0075, 0.015, and 0.030% w/v dodecyl maltoside and 0.75, 1.5 and 3.0% w/w polypeptide relative to synthetic polymer materials respectively.

### EXAMPLES NOS. 146-290

Solutions useful for producing a biocompatible membrane in accordance with the present invention can be prepared generally as described in Examples Nos. 1-145, respectively, however, the polypeptide solution mixed with the synthetic polymer can be a solution of 10 mg/ml of Succinate:ubiquinone oxidoreductase (Complex II) in water which also can include 0.15% Thesit (polyoxyethylene(9)dodecyl ether, C12E9) available from Roche, Indianapolis, Ind. This surfactant replaces, in general, the dodecyl maltoside in examples 1-145 in similar concentration.

### EXAMPLES NOS. 291-435

Solutions useful for producing a biocompatible membrane in accordance with the present invention can be prepared generally as described in Examples Nos. 1-145, respectively, however, the polypeptide solution used to dilute the synthetic polymer can be a solution of 10 mg/ml of Nicotinamide Nucleotide Transhydrogenase in water which also can include 0.15% Triton X-100. This surfactant replaces, in general, the dodecyl maltoside in examples 1-145 in similar concentration. Furthermore, in examples 1-145 which include dodecyl b-D-glucopyranoside, this detergent can be substituted with Nonidet P-40 in similar concentration.

### EXAMPLE 436

Membranes are formed on a dielectric perforated support. The support is made of KAPTON available from DuPont (1 mil thick) and is laser-drilled with apertures of 100 micrometers in diameter and 1 mil deep. The array of apertures can have a density as high as 1,700 apertures/cm2. A biocompatible membrane is formed across the apertures using the PEG 8000/PROTOLYTE A700 membrane described in detail previously. The resulting final solution containing the block copolymer, stabilizing polymer and polypeptide is then deposited onto the substrate in a manner that completely covered the apertures, dropwise by pipet, 4 microliters at a time. The solvent was allowed to evaporate at room temperature under a hood. The membrane-support assembly was stored in a vacuum chamber prior to use.

### EXAMPLES 437-464 (Proton-tunneling membranes)

For Examples 437-439, the following PTM solution was used.

A solution of 50 mg/ml of polystyrene-poly(1-4)butadiene-polystyrene (3G55 lot 7453064P, BASF, Ludwigshafen, Germany) in tetrahydrofuran (lot 15879CA, Aldrich, Milwaukee, Wis.) was prepared by dissolving the polymer pellets in the solvent in a glass bottle with continuous stirring.

### EXAMPLE 437

A Kapton (polyimide, Dupont) support was laser-drilled to create a set of 4 arrays of 100 apertures (10×10) of approximately 100 microns in diameter each, creating an open area of approximately 0.055 cm2 per array, or a total open area of 0.22 cm2 over an area with approximately 33% open area in the vicinity of the apertures. The Kapton was cut to approximately 1 inch by 1.75 inches, with the arrays in the center of the piece, and temporarily mounted over a larger aperture (approximately 1 inch) in a ⅜ in. thick piece of Lexan.

Using a micropipette device, 4 microliters of the polystyrene-poly(1-4)butadiene-polystyrene solution described above was applied to each array in such a manner as to cover all the apertures in the array. The solution was allowed to air dry in a chemical hood, then transferred to a vacuum oven and completely dried at room temperature for 15 minutes at what pressure.

The support with membrane was then removed from the oven, removed from the Lexan and immersed in 70% ethanol (30% deionized water) for 5 minutes.

The membrane was then tested by constructing the following test cell: a polysulfone plate (2″×3″×⅜″) served as the outer shell. A piece of zinc foil (1″×3″×0.25 mm, Goodfellow LS237171, Cambridge, England) served as the anode, followed by a silicone rubber gasket (⅜″ thickness) which sealed the anode compartment to the Kapton support/membrane, a second gasket (¼″ thickness) then sealed the support to the graphite cathode. (Poco, Decatur, Tex.) A final polysulfone plate, with the same size as the first plate, was fixed to the first plate via screws, clamping the layers together, but with the screws not in contact with either electrode, nor the anolyte or catholyte.

The anode compartment was then filled with a mixture of 0.8 ml of 2.3 M tetramethylammonium (TMA)-formate pH 8.0 and 0.2 ml of 2.6 TMA-OH (pH 15), the cathode compartment was filled with 1.0 ml of a solution of 100 millimolar TMA-sulfate, pH 7.0 supplemented with (final concentration) 0.1 N H2SO4 and 1% H₂O₂.

The anode and cathode were connected through a computer-controlled variably loaded circuit in parallel with an electrometer. Current and voltage were measured and were produced by the test cell in a manner consistent with the transfer of protons from the anode compartment through the membrane to the cathode compartment.

### EXAMPLE 438

A 1-mm thick polystyrene (with additional, unknown plasticizers) sheet was cut to a size of 1″×1.75″, and an aperture of 7/16″ diameter was punched at its center. The piece was fixed to a polytetrafluoroethylene (Teflon-Dupont) block of approximately 3″×3″×⅜″ with binder clips in such a manner that liquid, when deposited into the aperture, was unable to wick between the polyethylene and the Teflon pieces beyond a small distance (less than ¼ inch) away from the aperture.

A volume of 100 microliters of the above-described polystyrene-polybutadiene-polystyrene block copolymer solution was deposited in the aperture. The solvent was allowed to evaporate to dryness in a chemical fume hood, then the apparatus was transported to a vacuum hood, where the membrane was dried at room temperature for 15 minutes.

Following removal from the vacuum hood, the membrane-polystyrene support was delaminated mechanically from the Teflon block, incubated in 70% ethanol for 5 minutes, and was tested as described in Example 437. Voltage and current output of the test cell was consistent with proton transfer from the anode compartment to the cathode compartment.

### EXAMPLE 439

An aluminum casting block (3.25″×3″×½″) with a milled space of 2.25″×2″×20 mils was coated with a layer of polymer solution formed by mixing the above polystyrene-polybutadiene-polystyrene block copolymer solution (280 microliters) with 70 microliters of a solution of 50 mg/ml of poly(4-chlorostyrene) (P1351, Polymer Source, Dorval, Quebec) and 350 microliters of a suspension of 50 mg/ml of ultra-high molecular-weight polyethylene (UHMWPE) microparticles (GSI Exim America, Mason, Ohio; grade XM-221U, lot 19110A) in THF along with 1 ml of a suspension of 25 mg/ml dioctyl sulfosuccinate, sodium salt (lot 11312, Aldrich Chemical, Milwaukee, Wis.) in THF. The solvent was allowed to evaporate in a fume hood, then the membrane was dried in a vacuum oven, as in Example 437. The membrane was released from the casting mold by immersion in deionized water, then immersed in ethanol for 15 minutes.

The membrane was tested as in Example 437, however, the smaller plates were replaced with ones of 3.25″×3″ area, and a piece of sheet aluminum (Goodfellow, LS238505LC) served as the anode.

This assembly also produced voltage and current consistent with the transfer of protons from the anode to the cathode.

### EXAMPLE 440

A membrane was formed as in Example 437, above, however, the test cell was assembled, without first immersing the membrane in ethanol. The test cell failed to produce consistent voltage output, and produced no current output. This is consistent with the output produced by a complete dielectric between the anode and cathode, one in which no proton transfer occurs.

### EXAMPLE 441

A membrane was formed as in Example 437, above, with the additional inclusion of 1.6 microliters of an aqueous solution of 0.15% dodecyl maltoside (Sigma) in the polymer solution. Following vacuum drying the membrane was not immersed in ethanol prior to test cell assembly. However, the membrane produced current and voltage consistent with proton transfer through the membrane from the anode to the cathode.

### EXAMPLE 442

A membrane was formed as in Example 437, above, and was then immersed in a solution of 0.15% dodecyl maltoside instead of 70% ethanol, prior to assembly.

### EXAMPLE 443

A membrane was formed from a 50 mg/ml solution of a polystyrene-poly(1-4 butadiene)-polystyrene block copolymer (Polymer Source) as above in Example 437. When ethanol immersed, and assembled into a test cell as above, the material from this second manufacturer also demonstrated proton transfer.

### EXAMPLE 444

A membrane was formed as in Example 437, above, with a mixture of 80% (v/v) of the 50 mg/ml polystyrene-polybutadiene-polystyrene, block copolymer solution described above and 20% (v/v) of a solution of 50 mg/ml of poly(4-chlorostyrene) (Polymer Source).

### EXAMPLE 445

A membrane was formed as in Example 443, above, with the poly(4-chlorostyrene) replaced with poly(4-methylstyrene).

### EXAMPLE 446

A membrane was formed as in Example 443, above, with 60% (v/v) of the polystyrene-polybutadiene-polystyrene block copolymer solution and 40% (v/v) of the poly(4-chlorostyrene) solution.

### EXAMPLE 447

A membrane was formed on the same Kapton support as in Example 437, above, using a solution of 250 mg/ml of polystyrene. (250,000 mw, Polymer Source) Following ethanol immersion, and fuel cell assembly, this membrane also demonstrated proton transfer.

### EXAMPLE 448

A membrane was formed as in Example 437, above, using poly(1-4butadiene)-polystyrene-poly(1-4butadiene) as the polymer.

### EXAMPLE 449

A membrane was formed as in Example 437, above, using a polystyrene-poly(1-2butadiene)-polystyrene block copolymer. The test cell failed to produce consistent voltage output, and produced no current output. This is consistent with the output produced by a complete dielectric between the anode and cathode, one in which no proton transfer occurs.

### EXAMPLE 450

A membrane was formed as in Example 437, above. The membrane was immersed in isopropanol prior to test cell assembly.

### EXAMPLE 451

A membrane was formed as in Example 438, above, using poly 2-vinylnapthalene as the polymer.

### EXAMPLE 452

A membrane was formed as in Example 438, above, using poly(2-vinylpyridine)-poly(1-2butadiene)-poly(2-vinylpyridine) as the polymer. The test cell failed to produce consistent voltage output, and produced no current output. This is consistent with the output produced by a complete dielectric between the anode and cathode, one in which no proton transfer occurs.

### EXAMPLE 452

A membrane was formed as, in Example 450, and, following immersion in ethanol, was incubated in 1N sulfuric acid (Optima grade, Fisher Scientific, Pittsburgh, Pa.) overnight, then assembled into a test cell.

### EXAMPLE 453

A membrane was formed using the polystyrene support described in Example 438, above, with 100 microliters of the polymer-UHMWPE-surfactant solution described in 3, above.

### EXAMPLE 454

A membrane was formed as in Example 439, above, using 10 mg/ml of the polystyrene-polybutadiene-polystyrene block copolymer in THF alone.

### EXAMPLE 455

A membrane was formed as in Example 439, above, except that the dioctyl sulfosuccinate was replaced with an equal mass of Pluronic L-101 (BASF).

### EXAMPLE 456

A membrane was formed as in Example 439, above, except that the UHMWPE was replaced with an equal mass of corn starch (Argo).

### EXAMPLE 457

A membrane was formed as in Example 439, above, except that the UHMWPE was replaced with an equal mass of isotactic polystyrene (Polymer Source).

### EXAMPLE 458

A membrane was formed as in Example 437, above, and immersed in ethanol for 5 minutes. Following treatment, the membrane/support was mounted and sealed between two O-rings that separated two 20 milliliter compartments. The compartments were filled with 0.1 N sulfuric acid,on one side, and 0.1 N sodium hydroxide on the other side. The pH of the solutions on either side of the membrane was monitored. Over a 24-hour period, the pH on either side was unchanged.

### EXAMPLE 459

A membrane was formed using the block copolymer solution from example 437 with the following modification: to 14.4 microliters of the solution was added 1.6 microliters of a solution containing the following: 10 mg/ml E. coli Complex I, 50 mM MES pH 6.0, 50 mM NaCl, 0.15% dodecyl maltoside. The membrane was formed and tested as described in example 437. The proton flux produced was three times that of a standard PTM.

### EXAMPLE 460

A membrane was produced as in example 459, above, the membrane/support was mounted and sealed between two o-rings that separated two 20 milliliter compartments. The compartments were filled with 0.1 N sulfuric acid on one side, and 0.1 N sodium hydroxide on the other side. The pH of the solutions on either side of the membrane was monitored. Over a 24-hour period, the pH on either side was unchanged.

### EXAMPLE 461

A membrane was formed as in example 459, above, with the following exception: the solution which was added to the block copolymer formulation, containing the polypeptide, buffer, salt and surfactant was heated to 100 degrees Celsius in a boiling water bath for 10 minutes prior to mixing with the block copolymer. The membrane formed was tested as in example 1. The cell produced less output than a PTM with a similar amount of dodecyl maltoside surfactant, alone. (As in Example 5.)

### EXAMPLE 462

A membrane was formed as in example 449, above. To 14.4 microliters of the polystyrene-poly(1-2butadiene)-polystyrene block copolymer was added 1.6 microliters of a solution containing the following: 10 mg/ml E. coli Complex I, 50 mM MES pH 6.0, 50 mM NaCl, 0.15% dodecyl maltoside. The membrane was formed and tested as described in example 437. The test cell now produced current and voltage.

### EXAMPLE 463

A membrane was produced as in example 459, above. The membrane was immersed in 70% ethanol (30% deionized water) prior to test cell assembly and testing. The test cell produced less output than the untreated membrane.

### EXAMPLE 464

A membrane was produced as in example 439. Following removal from the cast, a 2-cm×2-cm square was cut from the membrane, and laminated across a 1-cm×1-cm opening in a laminating pouch (ABC Docuseal, Quartet Co. Skokie, Ill.) with a Docuseal-40 Laminator (Quartet). The laminated membrane was activated as in example 1 and tested for proton transfer activity, which was not found to be present.

A membrane was formed and laminated as in example 29, above. The laminated membrane was treated with a thin surface coating of a mixture of 50% acetone, 50% hexane. The solvent was then allowed to evaporate. This was found to return an appearance of a foam-like structure to the membrane, that then again exhibited proton transfer activity, albeit at a decreased level from the original membrane prior to lamination.

As a general phenomenon, all of the membranes tested in Examples 437-464 formed without the addition of surfactants were complete dielectrics in the absence of surface treatment with a wetting agent.

### EXAMPLE 465

(Production of an Analyte Sensor)

An analyte sensor can be constructed as follows:

Membranes can be formed on a dielectric perforated barrier, as a support, made of KAPTON available from DuPont (1 mil thick) that is laser drilled with aperatures of 100 micrometers in diameter and 1 mil deep. The aperatures can have a density as high as 1,700 aperatures/cm². A biocompatible membrane is formed across the aperatures using the PEG 8000/PROTOLYTE A700 membrane described in detail previously. The resulting final solution containing the block copolymer, stabilizing polymer and polypeptide is then deposited onto the substrate in a manner that completely covers the aperatures, dropwise by pipet, 4 microliters at a time. The solvent is allowed to evaporate at room temperature under a hood. The membrane-support assembly can then stored in a vacuum chamber prior to use.

A cell can be constructed from DELRAN plastic. The membrane-support assembly produced as described above can then be sealed in place within the fuel cell with rubber gaskets to form two compartments, an anode compartment and a cathode compartment. The anode and cathode compartments are then filled (20 ml in each) with an aqueous electrolyte (1M TMA-formate pH 10 in the anode compartment and 100 mM TMA-sulfate, pH 2.0, containing 1% hydrogen peroxide in the cathode compartment). A titanium foil anode can then be connected in parallel to an electronically varied load. A computer with an analog/digital board is used to measure current and voltage output. The circuit completed by wiring these elements to a graphite cathode electrode in the cathode compartment.

The titanium foil anode is immersed in the electrolyte of the anode compartment. Introduced into the anode (first) compartment is a 5% v/v methanol which is the analyte to be detected, 12.5 mM NAD+ is used as electron carrier, 1M hydroquinone is used as electron transfer mediator, yeast alcohol dehydrogenase (5,000 units), aldehyde dehydrogenase (10 units) and formate dehydrogenase (100 units) are used as soluble enzymes.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An analyte sensor for detecting an analyte comprising:
a first compartment adapted for introduction of said analyte;
a second compartment;
a barrier separating said first and second compartments, wherein said first compartment contains at least one component adapted to interact with said analyte, said interaction resulting in transport of a species across said barrier; and
a detector adapted to detect said transported species, or a derivative of said transported species, thereby detecting the presence of said analyte.

2. The sensor of claim 1, wherein said at least one component comprises at least one polypeptide, said polypeptide capable of interacting with said analyte or a derivative of said analyte to participate in a chemical reaction.

3. The sensor of claim 1 or claim 2, wherein said barrier is at least partially permeable to said transported species, wherein said at least one component is adapted to interact with said analyte.

4. The sensor of claim 3, wherein said permeability of said barrier is dependant on the size of said transported species.

5. The sensor of claim 3, wherein said permeability of said transported species is dependant on the charge of said transported species.

6. The sensor of claim 3, wherein said permeability of said transported species is dependant on the charge and size of said transport species.

7. The sensor of claim 1, wherein said detector is selected from the group consisting of current and voltage meters, gas chromatographs, liquid chromatographs, mass spectrometers, nuclear magnetic resonance detectors, infra-red, ultra-violet or Raman spectrophotometers, C, H, N, O detectors, moisture detectors, conductivity sensors, thermometers, oxygen sensors, biological oxygen sensors, pH detectors, colorimetric detectors, turbidity meters, and particle size and distribution detectors.

8. An analyte sensor for detecting an analyte comprising:
a first compartment adapted for introduction of said analyte;
a second compartment;
a barrier separating said first and second compartments, said barrier comprising a biological membrane associated with a polypeptide, wherein said polypeptide is capable of participating in the transport of a species across said barrier upon interaction with said analyte, or a derivative of said analyte; and
a detector adapted to detect said transported species, or a derivative of said transported species, thereby indicating the presence of said analyte, wherein said detector (i) is not attached to said membrane, or (ii) is separated from said membrane by 50 nm or more.

9. The sensor of claim 8, wherein said transported species is transported from said first compartment to said second compartment.

10. The sensor of claim 8, wherein said transported species is transported from said second compartment to said first compartment.

11. The sensor of claim 8, wherein said first compartment further comprises at least one component adapted to effect formation of said derivative of said analyte.

12. The sensor of claim 8, wherein said first compartment or second compartment further comprises at least one component adapted to effect formation of said derivative of said transported species.

13. The sensor of claim 8, wherein said detector is selected from the group consisting of current and voltage meters, gas chromatographs, liquid chromatographs, mass spectrometers, nuclear magnetic resonance detectors, infra-red, ultra-violet or Raman spectrophotometers, C, H, N, O detectors, moisture detectors, conductivity sensors, thermometers, oxygen sensors, biological oxygen sensors, pH detectors, colorimetric detectors, turbidity meters, and particle size and distribution detectors.

14. An analyte sensor for detecting an analyte comprising:
a first compartment adapted for introduction of said analyte;
a second compartment;
a barrier separating said first and second compartments, said barrier comprising a biocompatible membrane having at least one layer comprising synthetic polymer, wherein said membrane is associated with at least one polypeptide, wherein said polypeptide is capable of participating in the transport of a species across said barrier upon interaction with said analyte, or a derivative of said analyte; and
a detector adapted to detect said transported species, or a derivative of said transported species, thereby indicating the presence of said analyte.

15. The sensor of claim 14, wherein said synthetic polymer includes at least one block copolymer and at least one non-block polymer or copolymer.

16. The sensor of claim 14 or 15, wherein said transported species is a proton, said at least one polypeptide capable of participating in the transport of protons across said barrier.

17. The sensor of claim 16 wherein said transport generates at least about 10 picoamps/cm² of current density.

18. The sensor of claim 16 wherein said transport generates at least about 10 milliamps/cm² of current density.

19. The sensor of claim 14, wherein said at least one polypeptide is embedded in said at least one layer.

20. The sensor of claims 14 or 19 wherein said at least one polypeptide is a polypeptide complex.

21. The sensor of claim 14, wherein said at least one polypeptide is present in an amount of at least about 0.01% by weight of said biocompatible membrane.

22. The sensor of claim 14, wherein said at least one polypeptide is present in an amount of at least about 5% by weight of said biocompatible membrane.

23. The sensor of claim 14, wherein said at least one polypeptide is present in an amount of at least about 10% by weight of said biocompatible membrane.

24. The sensor of claim 14, wherein said synthetic polymer includes at least one block copolymer wherein said at least one block copolymer has a hydrophobic content that exceeds its hydrophilic content.

25. The sensor of claim 24 wherein said at least one block copolymer has at least one block having an average molecular weight of about 1,000 to about 20,000 Daltons.

26. The sensor of claim 25 wherein said at least one block copolymer has at least a second block having an average molecular weight of about 1,000 to about 20,000 Daltons.

27. The sensor of claim 24, wherein said at least one block copolymer is provided in an amount of at least about 35% by weight based on the weight of said biocompatible membrane.

28. The sensor of claim 24, wherein said at least one block copolymer is provided in an amount of about 35% to about 99% by weight based on the weight of said biocompatible membrane.

29. The sensor of claim 24, wherein said synthetic polymer comprises a plurality of block copolymers.

30. The sensor of claim 14 or 15 wherein said at least one polypeptide can participate in a redox reaction.

31. The sensor of claim 15, wherein said at least one polymer or copolymer has a molecular weight of about 5,000 to about 500,000 Daltons.

32. The sensor of claim 14, wherein said transported species is transported from said first compartment to said second compartment.

33. The sensor of claim 14, wherein said transported species is transported from said second compartment to said first compartment.

34. The sensor of claim 14, wherein said first compartment further comprises at least one component adapted to effect formation of said derivative of said analyte.

35. The sensor of claim 14, wherein said first compartment or second compartment further comprises at least one component adapted to effect formation of said derivative of said transported species.

36. The sensor of claim 14, wherein said detector is selected from the group consisting of current and voltage meters, gas chromatographs, liquid chromatographs, mass spectrometers, nuclear magnetic resonance detectors, infra-red, ultra-violet or Raman spectrophotometers, C, H, N, O detectors, moisture detectors, conductivity sensors, thermometers, oxygen sensors, biological oxygen sensors, pH detectors, colorimetric detectors, turbidity meters, and particle size and distribution detectors.

37. The sensor of any one of claims 1, 8 and 14, in which said sensor is disposable.

38. A detection device comprising:
a separation module adapted to separate a sample to be analyzed into at least two sample components;
a transfer element adapted to transfer at least one of said sample components into a first compartment of an analyte sensor as claimed in any of claims 1, 8 and 14.

39. The detection device of claim 38, wherein said separation module is adapted to subject said sample to a series of separations.

40. The detection device of claim 39, wherein said separation module comprises a plurality of separation devices.

41. The detection device of claim 40, wherein said separation device is selected from the group consisting of a cell sorter, chromatography column, and centrifuge.

42. A method of analyzing a sample for the presence of an analyte, comprising:
providing an analyte sensor as claimed in any of claims 1, 8 and 14;
introducing a sample containing said analyte into said first compartment; and
observing said detector for the presence of said analyte.

43. A sensor array for detecting at least one analyte comprising two or more analyte sensors as claimed in any of claims 1, 8 and 14.

44. The sensor array of claim 43, wherein said analyte sensors are adapted to detect at least two analytes.

45. An analyte sensor for detecting an analyte comprising:
a first compartment adapted for introduction of said analyte;
a second compartment;
a barrier separating said first and second compartments, said barrier comprising a membrane associated with at least one polypeptide wherein said polypeptide is capable of participating in the transport of a detected species across said barrier and wherein said polypeptide is adapted to interact with said analyte or a derivative of said analyte whereby transport of said detected species is reduced;
and a detector adapted to detect said reduction, thereby detecting the presence of said analyte.

46. The analyte sensor of claim 45 wherein said polypeptide is a pore forming polypeptide.

47. The analyte sensor of claim 46 wherein said polypeptide is gramaciden.

48. The analyte sensor of any of claims 45, 46 and 47 wherein said membrane is a biocompatible membrane having at least one layer comprising synthetic polymer.

49. The analyte sensor of claim 45 wherein the mass ratio of said polypeptide to polymer is about 1:100 to about 1:50,000.
